(19) United States
(12) Patent Application Publication
(10) Pub. No.: US 2009/0292119 A1
(43) Pub. Date: Nov. 26, 2009

(54) METHODS FOR SYNTHESIZING BENZOTHIAZEPINE COMPOUNDS

(75) Inventors: Donald W. Landry, New York, NY (US); Shixian Deng, White Plains, NY (US); Zhen Z. Cheng, Elmhurst, NY (US)

Correspondence Address: WilmerHale/Columbia University, 399 PARK AVENUE, NEW YORK, NY, 10022, US

(73) Assignee: THE TRUSTEES OF COLUMBIA UNIVERSITY IN THE CITY OF NEW YORK, New York, NY (US)

(21) Appl. No.: 12/263,435

(22) Filed: Oct. 31, 2008

Related U.S. Application Data
(63) Continuation-in-part of application No. 11/506,285, filed on Aug. 17, 2006.
Application No. 11/506,285 is a continuation-in-part of application No. 11/212,309, filed on Aug. 25, 2005.
Application No. 11/212,309 is a continuation-in-part of application No. 10/809,089, filed on Mar. 25, 2004.
Application No. 10/809,089 is a continuation-in-part of application No. 10/763,498, filed on Jan. 22, 2004, now abandoned.
Application No. 10/763,498 is a continuation-in-part of application No. 10/680,988, filed on Oct. 7, 2003, now abandoned.

(51) Int. Cl. C07D 417/02; C07D 285/36; C07D 413/02

(52) U.S. Cl. 540/552

(57) ABSTRACT

The present invention provides improved methods for synthesizing novel benzothiazepine compounds. In particular, the invention relates to a new method that generally is used to make the substituted 2,3,4,5-tetrahydro-1,4-benzothiazepine compounds of a general formula

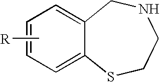

which then may be used to make many other benzothiazepine compounds.

### CROSS-REFERENCE TO RELATED APPLICATIONS

This application is continuation-in-part of U.S. patent application Ser. No. 11/506,285, filed Aug. 7, 2006, which is a continuation-in-part of U.S. patent application Ser. No. 11/212,309, filed on Aug. 25, 2005, which is a continuation-in-part of U.S. application Ser. No. 10/809,089, filed in Mar. 25, 2004, which is a continuation-in-part of U.S. application Ser. No. 10/763,498, filed on Jan. 22, 2004, now abandoned, which is a continuation-in-part of U.S. application Ser. No. 10/680,988, filed on Oct. 7, 2003, the contents of each of which are incorporated herein by reference thereto.

### FIELD OF THE INVENTION

The invention relates to methods of synthesizing benzothiazepine compounds. In particular, it relates to methods of synthesizing 1,4-benzothiazepines that are useful for treating cardiac, skeletal muscular and cognitive diseases and disorders.

### BACKGROUND OF THE INVENTION

The sarcoplasmic reticulum (SR) is a structure in cells that functions, among other things, as a specialized intracellular calcium (Ca²⁺) store. Channels in the SR called ryanodine receptors (RyRs) open and close to regulate the release of Ca²⁺ from the SR into the intracellular cytoplasm of the cell. There are three types of ryanodine receptors, all of which are highly-related Ca²⁺ channels: RyR1, RyR2, and RyR3. RyR1 is found predominantly in skeletal muscle as well as other tissues, RyR2 is found predominantly in the heart as well as other tissues, and RyR3 is found in the brain as well as other tissues. The RyR channels are formed by four RyR polypeptides in association with four FK506 binding proteins (FKBPs), specifically FKBP12 (calstabinl) and FKBP12.6 (calstabin2). Calstabinl binds to RyR1, calstabin2 binds to RyR2, and calstabinl binds to RyR3. The FKBP proteins (calstabin1 and calstabin2) bind to the RyR channel (one molecule per RyR subunit), stabilize RyR-channel functioning, and facilitate coupled gating between neighboring RyR channels, thereby preventing abnormal activation of the channel during the channel's closed state.

U.S. Pat. No. 6,489,125, and U.S. patent application Ser. Nos. 10/608,723 and 10/288,606, the contents of each of which are incorporated herein by reference thereto, disclose a novel mechanism underlying cardiac arrhythmias and methods of treatment thereof. In particular, it has been shown that exercise-induced cardiac arrhythmias and sudden cardiac death result from a reduced affinity between cardiac ryanodine receptor (RyR2) and its binding protein FKBP12.6, and that JTV-519 (4-[3-(4-benzylpiperidin-1-yl)propionyl]-7-methoxy-2,3,4,5-tetrahydro-1,4-benzothiazepine monohydrochloride; also known as k201 or ICP-Calstan 100) modulates the function of RyR calcium-ion channels by increasing FKBP12.6 binding to RyR2.

Additionally, U.S. Pat. No. 7,312,044 discloses that dissociation of FKBP12 from RyR1 channels may be be responsible for muscular disorders. Moreover, U.S. patent application Ser. Nos. 11/212,309, 11/212,413 and 11/506,285, disclose novel benzothiazepine compounds that modulate the function of RyR calcium-ion channels by increasing the binding of FKBP proteins to RyR channels, and are thereby useful in treating diseases associated with malfunction of RyR channels.

The method of synthesizing benzothiazepine compounds for example JTV-519 was disclosed by Kaneko et al. (U.S. Pat. No. 5,416,066; WO 92/12148; JP4230681), in which JTV-519 is prepared by reacting 7-methoxy-2,3,4,5-tetrahydro-1,4-benzothiazepine (a 1,4-benzothiazepine intermediate) with acryloyl chloride, and then reacting the resulting product with 4-benzyl piperidine.

Two processes for the preparation of 7-methoxy-2,3,4,5-tetrahydro-1,4-benzothiazepine and similar compounds have been previously reported in the literature. The first process, which is disclosed in U.S. Pat. No. 5,416,066 by Kaneko et al., involves a synthetic route of six steps that starts with 2,5-dihydroxybenzoic acid. In this process, 2,5-dihydroxybenzoic acid is selectively methylated with dimethyl sulfate. The resulting compound is then reacted with dimethylthiocarbamoyl chloride for 20 h, and then subjected to high temperature (270° C.) for 9 h. The product of this step is refluxed with sodium methoxide in methanol for 20 h followed by reacting with 2-chloroethylamine, under basic conditions and at a high temperature, to produce a cyclized amide. The cyclized amide is then reduced with LiAlH₄ to yield 7-methoxy-2,3,4,5-tetrahydro-1,4-benzothiazepine (a 1,4-benzothiazepine intermediate).

The second process for the preparation of 7-methoxy-2,3,4,5-tetrahydro-1,4-benzothiazepine is disclosed in Japanese patent No. JP 10045706 by Hitoshi. This process starts with reacting NaSMe with 2-bromo-5-methoxy benzaldehyde, and oxidizing the resulting product with chlorine, followed by reflux in water, to yield a disulfide dialdehyde. The dialdehyde is treated with 2-chloroethylamine, and the resulting product is reduced with a reducing agent, for example NaBH₄. The resulting compound is cyclized to give 7-methoxy-2,3,4,5-tetrahydro-1,4-benzothiazepine.

However, the first process, described in U.S. Pat. No. 5,416,066 by Kaneko et al., involves synthetic steps of high temperature and long reaction time. Additionally, it has been found that the thio group in the thiolated intermediate is easily oxidized by air to a disulfide compound, making it difficult to synthesize the subsequent cyclized product.

In view of the foregoing, there is a need to identify improved methods for synthesizing benzothiazepines which are novel modulators of RyR calcium-ion channels. The present invention satisfies the need of the industry.

### SUMMARY OF THE INVENTION

The present invention provides methods for synthesizing 1,4-benzothiazepine compounds and derivatives, which are modulators of RyR calcium-ion channels, and are therefore useful for the treatment of various diseases associated with RyR5, for example cardiovascular disease, muscle fatigue and cognitive disorders. In particular, the present invention provides methods for synthesizing unsubstituted or substituted 2,3,4,5-tetrahydro-1,4-benzothiazepine compounds of a general formula as described herein, including, but not limited to, S1, S2, S3, S4, S5, S6, S7, S9, S10 (or JTV519), S11, S12, S13, S14, S19, S20, S22, S23, S25, S26, S27, S36, S37, S38, S40, S43, S44, S45, S46, S47, S48, S49, S50, S51, S52, S53, S54, S55, S56, S57, S58, S59, S60, S61, S62, S63, S64, S66, S67, S68, S69, S70, S71, S72, S73, S74, S75, S76, S77, S78, S79, S80, S81, S82, S83, S84, S85, S86, S87, S88, S89, S90, S91, S92, S93, S94, S95, S96, S97, S98, S99, S100, S101, S102, S103, S104, S107, S108, S109, S110, S111, S112, S113, S114, S115, S116, S117, S118, S119, S120, S121, S122, and S123. These compounds may also be referred to by their corresponding ARM number. The structures of these compounds are disclosed in U.S. patent application Ser. Nos. 11/506,285, 11/212,309, and 10/809,089, the contents of each of which are incorporated by reference herein.

In particular, the invention relates to a new method that generally is used to make the compound represented by the formula:

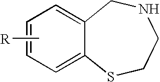

which then may be used to make the other compounds. When R is methoxy and is at position 7 of the benzothiazepine ring, the compound is designated 7-methoxy-2,3,4,5-tetrahydro-1,4-benzothiazepine or “S26”. When R is H, the compound is designated 2,3,4,5-tetrahydro-1,4-benzothiazepine or “S68”. S68 may be prepared in accordance with the methods of the present invention, or it may be commercially purchased on prepared in accordance with the methods as further described herein.

### DETAILED DESCRIPTION OF THE EMBODIMENTS

All publications cited herein are hereby incorporated by reference in their entireties.

To overcome problems of existing methods, the present invention relates to a novel process for making 2,3,4,5-tetrahydro-1,4-benzothiazepine for example 7-methoxy-2,3,4,5-tetrahydro-1,4-benzothiazepine and other derivatives from readily-available and inexpensive starting materials. This process simplifies isolation and purification steps, and may be used to prepare various 1,4-benzothiazepine compounds and derivatives. The various synthetic routes to the compounds are described herein.

In one embodiment, the present invention provides a method of synthesis comprising the steps of:
(a) treating a compound having the formula:

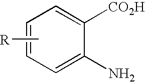

wherein R is H, OR₁, SR₁, N(R₁)₂, alkyl, or halide and R₁ is independently at each occurrence alkyl, aryl, or H, with a diazotizing agent and a disulfide under conditions sufficient to fotrm a compound having formula:

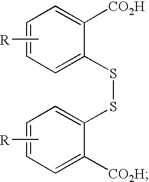

(b) treating the compound formed in (a) with a chloride and a chloroethylamine, under conditions sufficient to form a compound having formula:

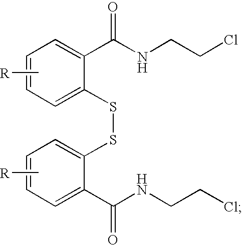

(c) treating the compound formed in (b) with a reducing agent and a base under conditions sufficient to form a compound having formula:

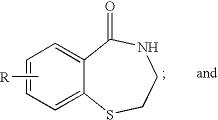

(d) treating the compound formed in (c) with a reducing agent under conditions sufficient to form a compound having formula:

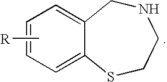

In one embodiment, the chloride is an acyl chloride, oxaloyl chloride or thionyl chloride.

In one embodiment. R is at position 7 of the benzothiazepine ring, and the compound formed in (d) has the structure (1):

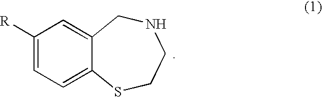

In accordance with this embodiment, the process comprises the following steps:
(a1) treating a compound having the formula:

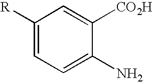

wherein R is as defined herein, with a diazotizing agent and a disulfide under conditions sufficient to form a compound having formula:

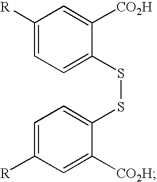

(b1) treating the compound formed in (a1) with a chloride and a chloroethylamine, under conditions sufficient to form a compound having formula:

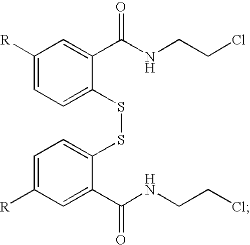

(c1) treating the compound formed in (b1) with a reducing agent and a base under conditions sufficient to form a compound having formula:

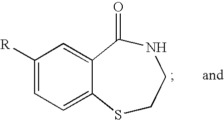

(d1) treating the compound formed in (c1) with a reducing agent under conditions sufficient to form a compound having formula:

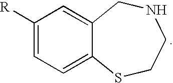

In another embodiment, the diazotizing agent in reaction (a) or (a1) is NaNO₂. In yet another embodiment, the disulfide in reaction (a) or (a1) is Na₂S₂. In yet another embodiment, the chloride in reaction (b) or (b1) is SOCl₂. In yet another embodiment, the reducing agent in reaction (c) or (c1) is trimethylphosphine (PMe₃) or sodium dithionite. In yet another embodiment, the base in reaction (c) or (c1) is triethyl amine, a carbonate for example sodium, potassium or cesium carbonate, sodium or potassium hydride, diisopropylamine (DIPEA), N-methylmorpholine (NMM), and the like. In yet another embodiment, the reducing agent in reaction (d) or (d1) is LiAlH₄.

In one embodiment, the compound in step (a) having the formula:

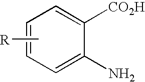

is synthesized by treating a compound having the formula:

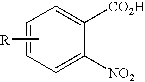

with a reducing agent for example H₂ in the presence of a catalyst for example Pd/C, platinum oxide, or raney nickel; or with iron or zinc in the presence of acid, under conditions sufficient to form the compound in (a).

In another embodiment, the method of the invention comprises any one of the following steps (e1), (f1), (g1) or (h1):
(e1) reacting the compound formed in (d) with a compound of formula R₂SO₂Cl under conditions sufficient to form a compound of the formula:

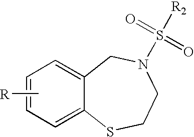

wherein R₂ is CH₂═CH—, Me, p-Me-C₆H₄, or —NH-2-Pyridyl.

In one embodiment, compounds S3, S4, S6 and S54 may be prepared from S26 in accordance with step (e1) by reacting S26

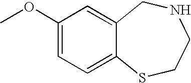

with R₂SO₂Cl, wherein R² is CH₂═CH—(S3), Me—(S4), p-Me-C₆H₄— (S5), or NH-2-Py (S54).
(f1) reacting the compound formed in (d) with a compound of formula R₂SO₂Cl under conditions sufficient to form a compound of the formula:

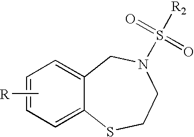

wherein R₂ is —CH₂═CH—; and reacting the compound thus formed with a compound of formula HNR₃R₄ wherein NR₃R₄ is

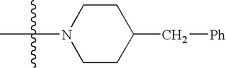

or —NBu₂, under conditions sufficient to form a compound of the formula:

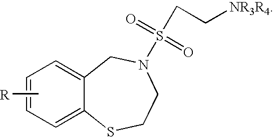

In one embodiment, compounds S1 and S2 may be prepared from compound S3 in accordance with step (f1), by reacting S3 with HNR₃R₄, where NR₃R₄ is

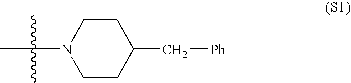

or —NBu₂ (S2). In another embodiment, compound S82 may be prepared in a manner analogous to the preparation of S1, but starting from the desmethoxy compound S68.
(g1) reacting the compound formed in (d) with SO₂Cl₂ and a base under conditions sufficient to form a compound of the formula:

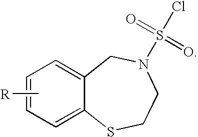

(h1) reacting the compound formed in (d) with SO₂Cl₂ and a base under conditions sufficient to form a compound of the formula:

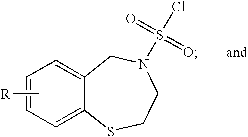

hydrolyzing that compound under conditions sufficient to obtain a compound of the formula:

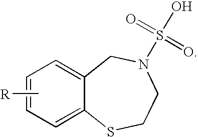

In another embodiment, compound S112 may be prepared from S26 in accordance with step (h1), by adding SO₂Cl₂ to a mixture of S26 and pyridine. After removal of the solvents, the residue is dissolved in a suitable basic solution, for example NaOH, and acidified, for example with 1N HCl.

In yet another embodiment, the method of the invention further comprises any one of the following steps (e2), (f2) or (g2):
(e2) reacting the compound formed in (d) with a compound of formula R₅COX₁, wherein R₅ is —CH₂I, Ph-, CH₂═CH—, 4-N₃-2-OH—C₆H₅ or

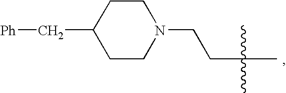

and X₁ is —Cl or —OSu, under conditions sufficient to form a compound of the formula:

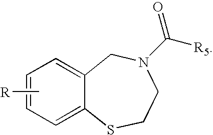

In one embodiment, compounds S7, S9, S27 and S40 may be prepared from the compound S26 in accordance with step (e2), by reacting S26 with a compound of formula R₅COX₁, where X₁ is —Cl or —OSu and R₅ is ICH₂— (S27), Ph- (S9), CH₂═CH— (S27), or 4-N₃-2-OH—C₆H₅ (S40). In another embodiment, the desmethoxy compound S83 may be prepared in a similar method, by reacting S68 with a compound of formula R₅COX₁, wherein R₅ is:

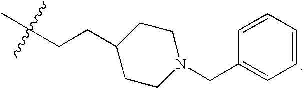

(f2) forming the compound

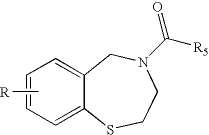

by the method of (e2) and treating that compound with hydrogen peroxide or m-CPBA under conditions sufficient to form a compound of the formula:

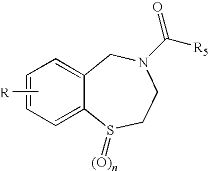

wherein n is 1 or 2. In one embodiment, compounds S20 and S23 may be prepared from a compound of formula

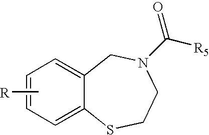

in accordance with step (f2), by treating such compound where R₅ is CH₂═CH— (S20) or

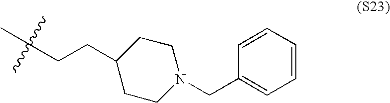

with H₂O₂. If necessary, sodium thiosulfate also is used to treat the intermediate.
(g2) forming the compound

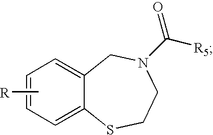

by the method of (e2), wherein R₅ is CH₂═CH—, and reacting that compound with a compound of formula HNR₈R₉, wherein NR₈R₉ is

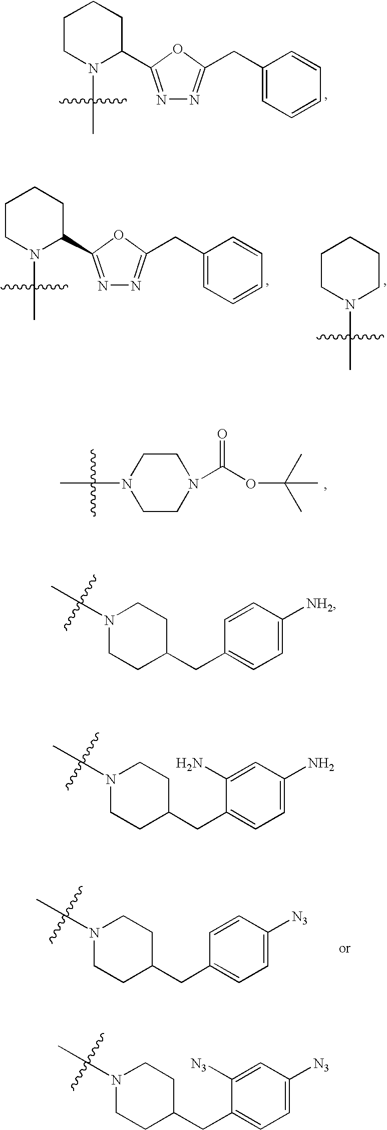

under conditions sufficient to form a compound of the formula:

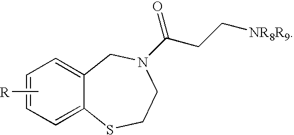

In one embodiment, compounds S55, S56, S58 and S60-63 may be prepared in accordance with step (g2), by reacting S27, i.e., S26-COR₅ wherein R₅ is CH₂═CH—, with a compound of formula HNR₈R₉, wherein NR₈R₉ is:

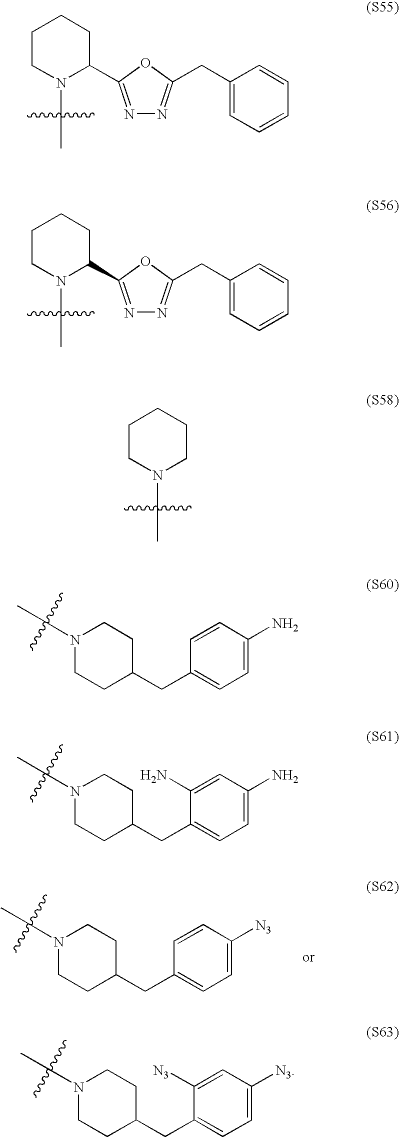

In yet another embodiment, the method of the invention further comprises any one of the following steps (e3), (f3), (g3), (h3), (i3), (j3) or (k3):
(e3) reacting the compound formed in (d) with an acid chloride of formula Cl—C(═O)C(═O)ORₐₐ under conditions sufficient to form a compound of the formula:

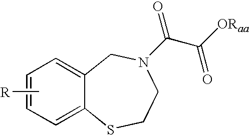

wherein Rₐₐ, is C₁-C₄ alkyl or aryl.
(f3) reacting the compound formed in (d) with an acid chloride of formula Cl—C(═O)C(═O)ORₐₐ under conditions sufficient to form a compound of the formula:

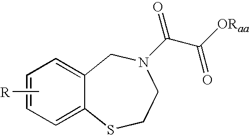

wherein Rₐₐ is C₁-C₄ alkyl or aryl; and reacting that compound with an acid or a base under conditions sufficient to form a compound of the formula:

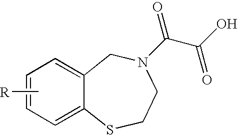

or a salt thereof.
(g3) reacting the compound formed in (d) with an acid chloride of formula Cl—C(═O)C(═O)ORₐₐ under conditions sufficient to form a compound of the formula:

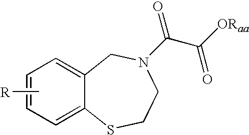

wherein Rₐₐ, is C₁-C₄ alkyl or aryl; and reacting that compound with an acid or a base under conditions sufficient to form a compound of the formula:

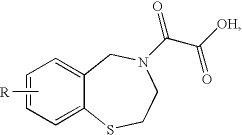

or a salt, and in the case a salt is formed, contacting the salt with an acid under conditions sufficient to form the compound of formula

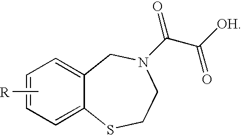

In some embodiments, compounds S36, S57, S76 and S77 may be prepared in accordance with these steps (e3), (f3) or (g3). S57 may be prepared by reacting S26 with methyl chlorooxoacetate. S76, the desmethoxy analog of S57, may be prepared in an analogous way, starting from the desmethoxy starting material S68. S36 may be prepared from S57 by reaction with sodium hydroxide. If desired, the aqueous phase from the basic washes is acidified and the product is extracted therefrom. S77, the desmethoxy analog of S36, may be prepared in an analogous way, from the desmethoxy derivative S76.
(h3) forming the compound

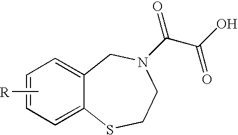

by the method of (f3) or (g3) and reacting that compound with hydrogen peroxide under conditions sufficient to form a compound of the formula:

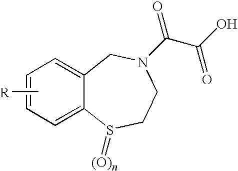

or its salts, wherein n is 1 or 2. In one embodiment, compound S104 may be prepared by reacting S36 with hydrogen peroxide in accordance with step (h3).
(i3) forming the compound

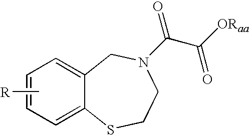

by the method of (f3) or (g3), wherein R is methoxy and Rₐₐ is methyl, and treating that compound with BBr₃ under conditions sufficient to form a compound of formula:

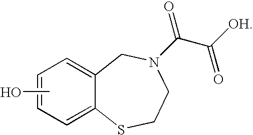

In one embodiment, compound S117 may be prepared from S57 in accordance with step (i3), by reacting S57 with BBr₃.
(j3) forming the compound

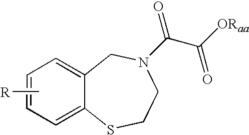

by the method of (f3) or (g3), and treating that compound with thionyl chloride or oxalyl chloride under conditions sufficient to form a compound of the formula:

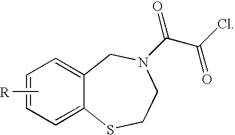

(k3) forming the compound

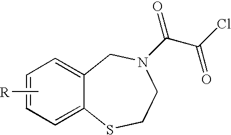

by one of the methods of (j3), and reacting that compound with:
(1) cystamine and a base under conditions sufficient to form a compound of the formula:

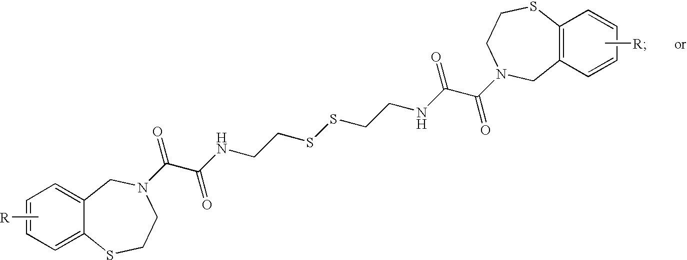

(2) a compound of the formula HX₂, wherein X₂ is —OCH₃, —NHEt, —NHPh, —NH, or —NHCH₂-4-pyridine, under conditions sufficient to form a compound of the formula:

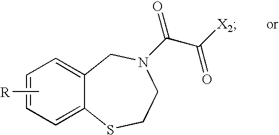

(3) cystamine and a base to form S36-cystamine- and reacting the S36-cystamine with an NHS activated ester of an azido compound under conditions sufficient to form a compound of the formula:

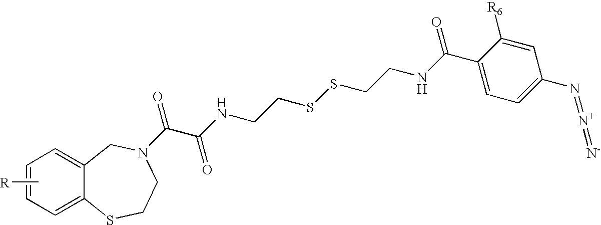

wherein R₆ is —NO₂ or —OH.

In one embodiment, compound S44 may be prepared from S36 in accordance with step (k3)(1), by treating S36 with thionyl chloride to form S36-Cl, which is reacted with cystamine. A base for example pyridine is used if desired, and the reaction mixture can be quenched with a basic solution (e.g., saturated sodium bicarbonate).

In another embodiment, compounds S57 and S59 may be prepared from S36-Cl in accordance with step (k3)(2), by reacting S36-Cl, with methanol (S57) or ethylamine (S59). Similarly, compounds S78-S81 are prepared from S77-Cl, by dissolving this compound in a solvent, and reacting with HX₂, where X₂ is —NHEt (S78), —NHPh (S79), —NH₂ (S80), and —NHCH₂-4-pyridine (S81).

In another embodiment, compounds S43 and S45 may be prepared from S36-cystamine in accordance with step (k3)(3), by reacting S-36 cystamine with an NHS activated ester of an appropriate azido compound.

In some embodiments, (i) Rₐₐ is methyl or ethyl; or (ii) the acid in reaction (f3) or (g3) is trifluoroacetic acid (TFA) or hydrochloric acid (HCl); or (iii) the base in reaction (f3′) is NaOH, KOH or LiOH; or (iv) R is methoxy; or (v) R is at position 7 of the benzothiazepine ring and the compound has the structure:

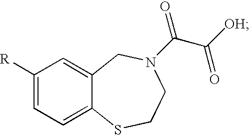

or (vi) the compound of formula

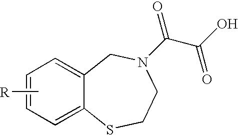

is further purified by dissolving the compound in water to form an aqueous solution; washing the aqueous solution with an organic solvent; acidifying the aqueous solution; and extracting the compound from the aqueous solution using an organic solvent to form an extract; and removing the organic solvent from the extract.

In yet another embodiment, the method of the invention further comprises any one of the following steps (e4)(1), (e4)(2), (f4), (g4), (h4) or (i4):
(e4)(1) reacting the compound formed in (d) with 4-nitrophenyl chloroformate under conditions sufficient to form a compound of the formula:

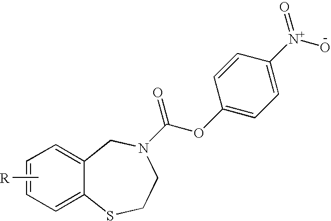

In one embodiment, compound S37 may be prepared from S26 in accordance with step (e4)(1), by reacting S26 with 4-nitrophenyl chloroformate (NO₂C₆H₅OCOCl). If desired, a base catalyst for example triethylamine may be used.
(e4)(2) reacting the compound formed in (d) with triphosgene and a base to form a compound of the formula

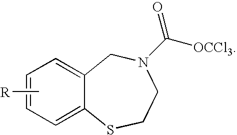

(f4) reacting the compound formed in (d) with:
   (1) 4-nitrophenyl chloroformate under conditions sufficient to form a compound of the formula:

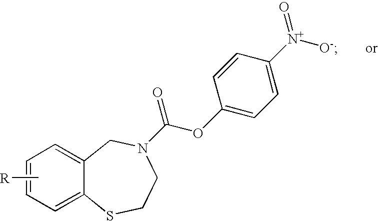

(2) triphosgene and a base to form a compound of the formula

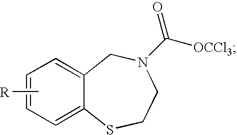

reacting either compound with an amine of formula HNR₇ₐR_{7b}, wherein NR₇ₐR_{7b} is —NH₂, —NEt₂, —NHCH₂Ph, —NHOH,

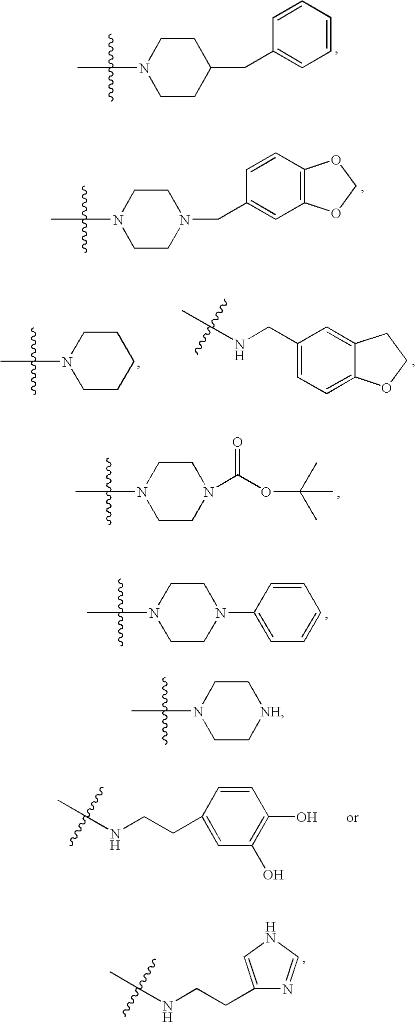

under conditions sufficient to form a compound of the formula:

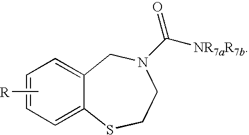

In some embodiments, compounds S6, S46-53, S64, S66, S67 and S114 may be prepared from S26 in accordance with steps (e4)(1), (e4)(2) and (f4), by forming S37- or S26-nitrophenylcarbamate or phosgene as described in steps (e4)(1) and (e4)(2), respectively, and reacting either compound with an amine of formula HNR₇ₐR_{7b}, wherein NR₇ₐR_{7b} are:

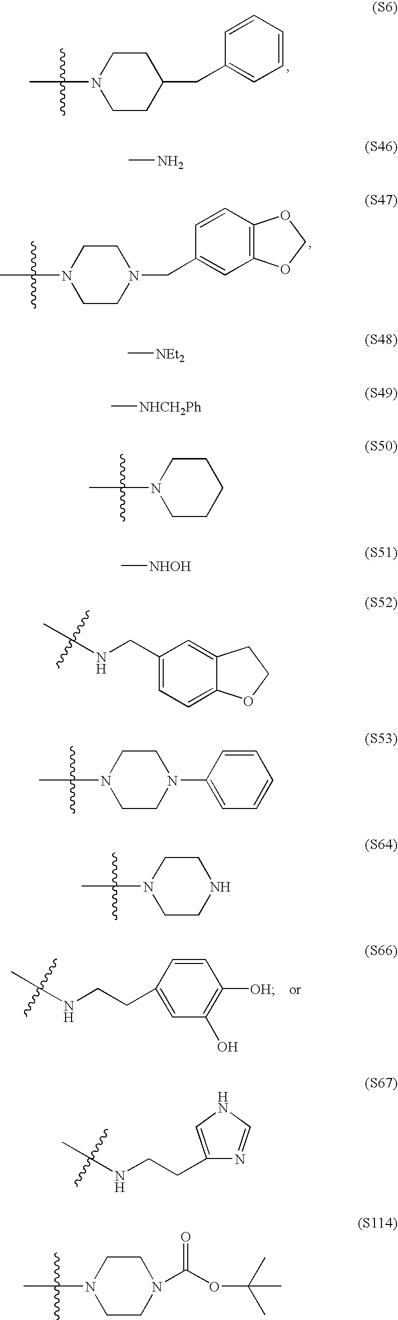

The analogous compounds S69-75 may be prepared by similar methods outlined above for S6, S46-53, S64, S66 and S67, starting from S68, the desmethoxy analog of S26.

In another embodiment, compounds S101, S102 and S103 may be prepared from S68 in an analogous way, by reacting S68 with 4-nitrophenyl chloroformate or triphosgene, as described in steps (e4)(1) and (e4)(2) respectively, and reacting either product with an amine of formula HNR₇ₐR_{7b}, wherein NR₇ₐR_{7b} are

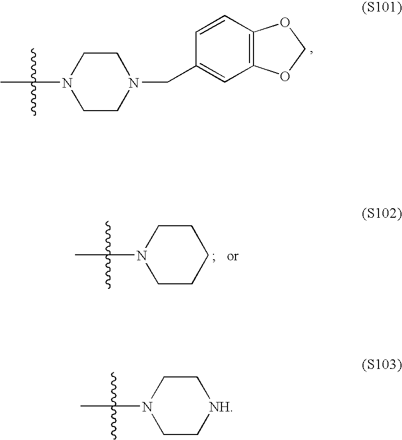

(g4) reacting an amine of formula HNR₇ₐR_{7b} wherein NR₇ₐR_{7b} is as defined herein with triphosgene under conditions sufficient to form a compound of formula Cl₃CO(C═O)NR₇ₐR_{7b}, and reacting that compound with the compound formed in (d) under conditions sufficient to form a compound of the formula:

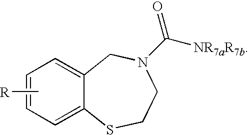

In one embodiment, compounds S6, S46-53, S64, S66, S67 and S114 may be prepared from S26 in accordance with step (g4), wherein an amine of formula HNR₇ₐR_{7b}, wherein NR₇ₐR_{7b} are as defined in (f4), is reacted with triphosgene, and the obtained compound of formula Cl₃CO(C═O)NR₇ₐR_{7b} is reacted with S26. The analogous compounds S69-75 may be prepared by similar methods to those outlined for S6, S46-53, S64, S66 and S67, starting from S68, the desmethoxy analog of S26. Compounds S101, S102 and S103 may be prepared in an analogous method, by reacting an amine of formula HNR₇ₐR_{7b}, wherein NR₇ₐR_{7b} are

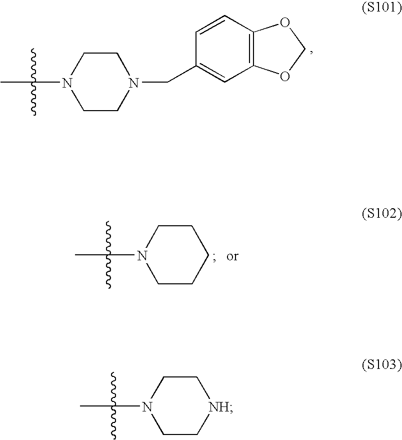

with triphosgene, and reacting the obtained compound of formula Cl₃CO(C═O)NR₇ₐR_{7b} with S68.
(h4) preparing the compound

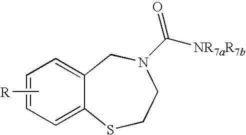

by the methods of (f4) or (g4), wherein NR₇ₐR_{7b} is

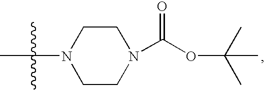

and reacting that compound with a Lawesson's Reagent under conditions sufficient to form a compound of the formula

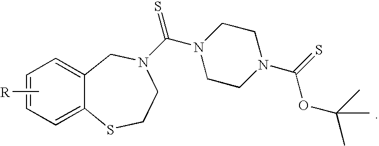

In one embodiment, compound S115 may be prepared from S114 in accordance with step (h4), by treating S114 with Lawesson's Reagent.
(i4) preparing the compound

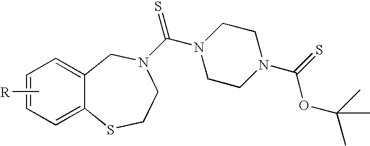

by the method of (h4) and reacting that compound with an acid under conditions sufficient to form a compound of the formula:

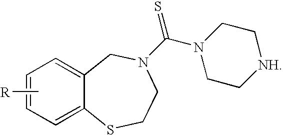

In another embodiment, compound S116 may be prepared from S115 in accordance with step (i4), by reacting S115 with an acid for example trifluoroacetic acid (TFA). In yet another embodiment, the method of the invention further comprises any one of the following steps (e5), (f5), (g5), (h5), (i5), (j5), (k5), (l5), (m5), (n5), (o5), (p5), (q5), (f5), (s5) or (t5):
(e5) reacting the compound formed in (d), wherein R is at position 7 of the benzothiazepine ring, with Boc₂O or Boc-Cl under conditions sufficient to form a compound of the formula:

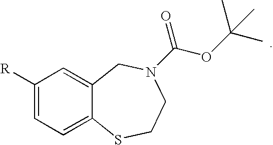

(f5) forming the compound

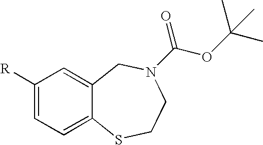

by the method of (e5), wherein R is methoxy, and reacting that compound with BBr₃ under conditions sufficient to form a compound of the formula:

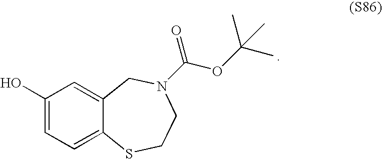

(g5) forming the compound S86 by the method of (f5) and reacting that compound with trifluoromethylsulfonyl anhydride under conditions sufficient to form the compound S87

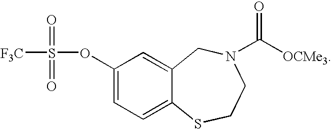

(h5) forming the compound S87 by the method of (g5) and reacting that compound with tris(dibenzylideneacetone)dipalladium(0), 2-(di-tert-butylphosphino)-biphenyl, and K₂PO₃, under conditions sufficient to form the compound S88

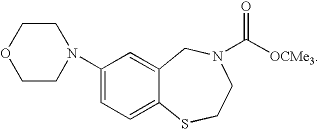

(i5) forming the compound S87 by the method of (g5) and reacting that compound with benzenethiol, optionally with a catalyst, under conditions sufficient to form compound S89

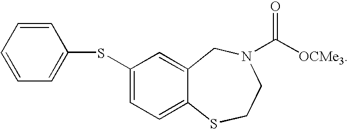

(j5) forming the compound S87 by the method of (g5) and reacting that compound with a base phenylboronic acid, and a catalyst, under conditions sufficient to form compound S90

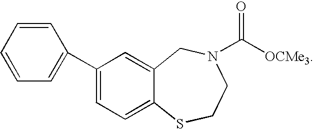

(k5) forming the compound S87 by the method of (g5) and reacting that compound with propene in the presence of a metal catalyst, a ligand and a base, under conditions suitable to form the compound S91

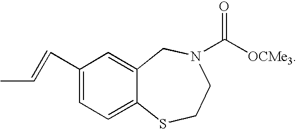

(l5) forming the compound S87 by the method of (g5) and reacting that compound with zinc cyanide and a catalyst, under conditions sufficient to form compound S92

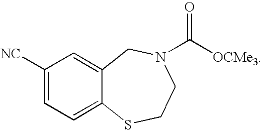

(m5) forming the compound S87 by the method of (g5) and reacting that compound with benzylamine in the presence of a catalyst, a ligand and a base, under conditions suitable to form the compound Sg3

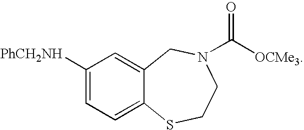

(n5) forming the compound S86 by the method of (f5) and reacting that compound with acetic anhydride under conditions sufficient to form compound S94

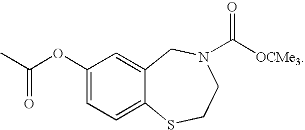

(o5) forming the compound S94 by the method of (n5) and reacting that compound with AlCl₃, and reattaching the Boc group, under conditions sufficient to form compound S95

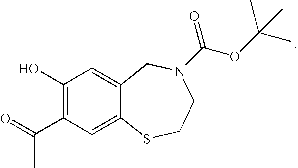

(p5) forming the compound S86 by the method of (f5) and reacting that compound with NaI and Chloramine-T under conditions sufficient to form compound S96

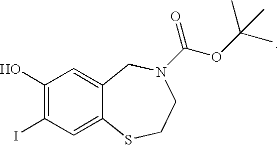

(q5) forming the compound S86 by the method of (f5) and reacting that compound with H₂SO₄ to form a sulfuric acid mixture, then adding HNO₃ to the sulfuric acid mixture thus formed, and reattaching the Boc group, under conditions sufficient to form the compound S97

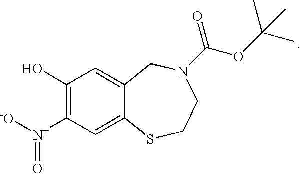

(r5) forming the compound S97 by the method of (q5) and hydrogenating that compound under conditions sufficient to form compound S98

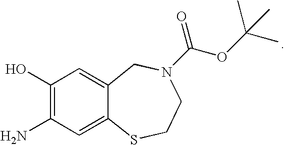

(s5) forming the compound S98 by the method of (r5) and reacting that compound with sodium nitrite and NaBaF₄ under conditions sufficient to form compound S99

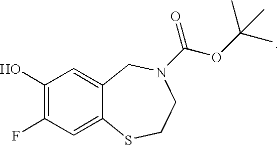

(t5) forming the compound S98 by the method of (r5), dissolving that compound in an acidic solution to form a mixture, and then reacting the mixture with sodium nitrite and NaN₃, and reattaching the Boc group under conditions sufficient to form compound S100

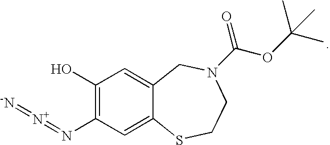

In one embodiment, S85 is prepared from S26 in accordance with step (e5), by reacting S26 with di-tert-butyl dicarbonate in a solvent. A base catalyst for example triethylamine also is used, if necessary.

In another embodiment, S86 may be prepared from S85, by treating S85 with BBr₃.

In one embodiment, S87 is prepared by reacting S86 with trifluoromethylsulfonyl anhydride. The reaction is carried out in a solvent, for example an organic solvent. A base catalyst for example triethylamine is added if necessary. When triethylamineis added, the reaction mixture formed by mixing the reactants and the solvent is quenched with water, after which the aqueous-layer is extracted with an appropriate organic solvent. If desired, the organic layers are dried (e.g., using magnesium sulfate), and the organic layers are concentrated. Purification of the concentrated organic layers yields S87.

In one embodiment, S88 is prepared from S87 by reaction with morpholine, tris(dibenzylideneacetone)dipalladium(0), 2-(di-tert-butylphosphino)-biphenyl, and potassium phosphate. The reaction mixture is diluted with a solvent, for example methylene chloride or another appropriate organic solvent, and washed with water. The aqueous layer, formed by washing with water, is extracted with an organic solvent, for example methylene chloride. The organic layers are then dried (e.g., over magnesium sulfate) and concentrated. The residue is purified, for example by silica gel flash chromatography, to yield S88.

In one embodiment, S89 is prepared from S87 by reaction with benzenethiol and i-Pr₂NEt in a solvent, for example CH₃CN or another appropriate organic solvent. After reaction, an organic solvent for example ethyl acetate is added to the reaction mixture. If necessary, the reaction mixture is washed with one or more of acidic (e.g. HCl), basic (e.g. NaOH), and water solutions. After drying (e.g. with Na₂SO₄), the solution is concentrated. Purification, for example by chromatography, yields S89. In an alternative, refluxing S87 with benezethiol in an appropriate solvent for example dioxane with a catalyst for example i-Pr-NEt/Pd₂(dba)₃/xantphos yields S89.

In one embodiment, S90 is prepared from S87 reacted with a base, phenylboronic acid, and a catalyst. In one embodiment, the base is K₂CO₃ and the catalyst is Pd(Ph₃P)₄. In one embodiment, the reaction occurs in a solvent, for example an organic solvent, for example dioxane. The reaction mixture formed by mixing the reactants and the solvent is diluted with a solvent (e.g. methylene chloride), and washed with water to remove unwanted hydrophilic compounds. Concentration and purification of the residue yields S90.

In one embodiment, S91 is prepared under Heck conditions by treating S87 with propene in the presence of a metal catalyst e.g., Pd catalyst, a ligand and a base in a suitable solvent and temperature.

In one embodiment, S92 is prepared from S87 reacted with zinc cyanide. The reaction occurs in a solvent, for example an organic solvent like DMF. A catalyst for example Pd(Ph₃P)₄ is also used to facilitate and hasten the reaction. The reaction mixture formed by mixing the reactants and the solvent, if necessary, is diluted with water and an acidic solution and extracted with an organic solvent. The organic extracts then are washed using a salt solution, dried, filtered, and concentrated. Purification of the residue proceeds, for example, by silica gel column chromatography.

In another embodiment, S93 amay be prepared from S87 by treating S87 with benzylamine in the presence of a catalyst. e.g., Pd, a suitable ligand (e.g., phosphine), and a suitable base (e.g., sodium tert-butoxide) in an appropriate solvent and temperature. In one embodiment, S94 is prepared from S86 by reaction with acetic anhydride. The reaction takes place in a solvent, for example an organic solvent like methylene chloride. Triethylamine or another base catalyst is added as necessary. Washing with water, followed by drying (e.g. with sodium sulfate), is used as desired. Purification of the residue yields S94.

In one embodiment, S95 is prepared from S94 by reaction with anhydrous AlCl₃, in a solvent if desired. The solvent is an organic solvent like benzene. The reaction mixture is refluxed and cooled on ice. Extraction with an organic solvent, concentration, purification of the residue, and reaction of the intermediate with Boc₂O or Boc-Cl yields S95.

In one embodiment, S96 is prepared from S86 by iodination. For example, S86 is added to a solvent, for example an organic solvent like methanol, with excess NaI and Chloramine-T. The reaction mixture is quenched with Na₂S₂O₃ solution. Concentration and purification of the residue yields S96 as a mixture of mono-iodinated and di-iodinated products.

In one embodiment, S97 is prepared from S86 by reaction with a nitric acid. S86 is protected (e.g., using the Boc protecting groups) and added to concentrated sulfuric acid. Nitric acid is added to the reaction mixture. The reaction mixture is cooled and neutralized (e.g., using Na₂CO₃) to quench the reaction. Reaction of the intermediate with Boc₂O or Boc-Cl followed by organic extraction, subsequent concentration, and purification yields S97.

In one embodiment, S98 is prepared by hydrogenation of S97. For example, S97 is added to a solution, for example an organic solution like methanol. H₂ gas is bubbled through the solution and Pd/C catalyst or another applicable catalyst is added. Filtration to remove the catalyst and purification yields S97.

In one embodiment, S100 is prepared from S98. S98 is dissolved in acid solution, for example aqueous HCl. To this a solution of sodium nitrite, and then NaN₃ in water, are added. The reaction mixture is extracted using an organic solvent. If needed, the extract is washed with a basic solution (e.g., saturated sodium bicarbonate) and water, treated with Boc₂O or Boc-Cl, then washed with a basic solution. Organic layers from the washing are dried using, for example, anhydrous sodium sulfate, and concentrated to form a residue. The residue is purified to yield S100. To prepare S99, NaN₃ is substituted with NaBF₄ in a similar manner.

In yet another embodiment, the method of the invention further comprises any one of the following steps (e6) or (f6):
(e6) reacting the compound formed in (d) with a compound of formula

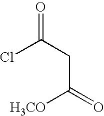

to produce a compound of formula:

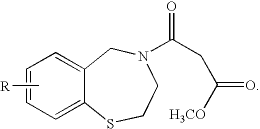

(f6) forming the compound

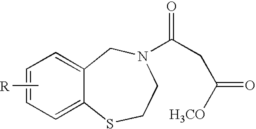

by the method of (e6) and reacting that compound with an acid or a base under conditions sufficient to form a compound of the formula:

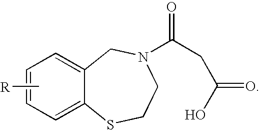

In one embodiment, these steps (e6) and (f6) may be used to prepare the compound S38 using

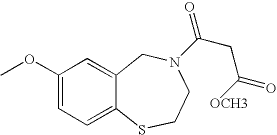

as the starting material, by treating such compound with an acid or a base to hydrolyze the ester.

In yet another embodiment, the method of the invention further comprises any one of the following steps (e7), (f7) or (g7):
(e7) reacting the compound formed in (d) with formaldehyde (CH₂O) and a reducing agent under conditions sufficient to form a compound of the formula:

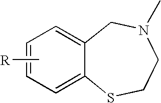

In one embodiment, compound S107 may be prepared from S26 in accordance with step (e7), by reacting S26 with formaldehyde and sodium cyanoborohydride. The reaction mixture can be maintained at around pH 4-5, for example by addition of a few drops of 1N HCl.
(f7) forming the compound

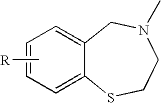

by the method of (e7) and reacting that compound with CH₃X₃ under conditions sufficient to form a compound of the formula:

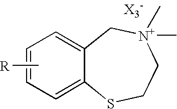

wherein X₃ is a halogen selected from F, Cl, Br and I. In one embodiment, compound S113 may be prepared from S107 in accordance with step (f7), by reacting S107 in ethyl acetate with CH₃I.
(g7) forming the compound

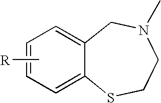

by the method of (e7) and reacting that compound with hydrogen peroxide under conditions sufficient to form a compound of the formula:

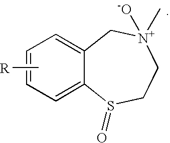

In one embodiment, compound S119 may be prepared from S107 in accordance with step (g7), by reacting S107 with H₂O₂ (for example around 50% solution), and an alcohol (for example MeOH).

In some embodiments, R is methoxy and is located at position 7 of the benzothiazepine ring, or the reducing agent in (a) is sodium cyanoborohydride (NaBCNH₃) or sodium triacetoxyborohydride.

In yet another embodiment, the method of the invention further comprises any one of the following steps (e8) or (f8):
(e8) reacting the compound formed in (d) with N-benzyloxycarbonyl-glycine (Cbz-Gly) under conditions sufficient to form a compound of the formula:

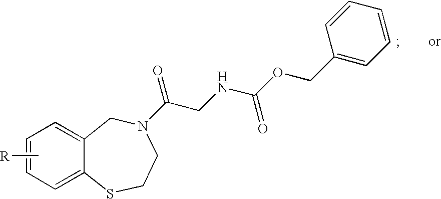

or
(f8) forming the compound

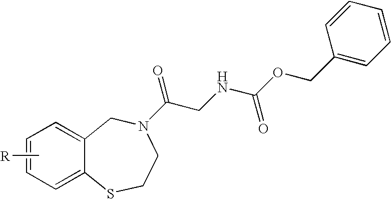

by the method of (e8) and treating that compound with an acid under conditions sufficient to form a compound of the formula:

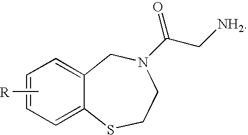

In some embodiments, compounds S108 and S109 may be prepared from S26 in accordance with these steps (e8) and (f8), by reacting a mixture of N-benzyloxycarbonyl-glycine (Cbz-Gly,), Diisopropyl-carbodiimide (DIC), and N-hydroxysuccinimide (NHS), and adding S26 to the reaction mixture.

S109 may be prepared from S108, by reacting S108 with an acid, for example HBr/CH₃CO₂H.

In yet another embodiment, the method of the invention further comprises any one of the following steps (e9) or (f9):
(e9) reacting the compound formed in (d) with a compound of formula:

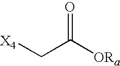

wherein X₄ is a halogen or a sulfonate and Rₐ is a C₁-C₄ alkyl under conditions sufficient to form a compound of the formula:

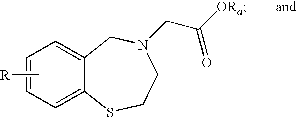

and
(f9) forming the compound

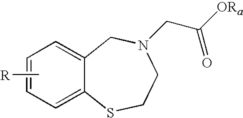

by the method of (e9) and treating that compound with an acid or a base under conditions sufficient to form a compound of the formula:

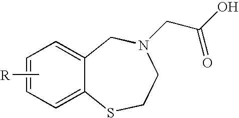

or a salt thereof

In some embodiments, the compounds S110 and S111 may be prepared from S26 in accordance with steps (e9) and (f9). S110 may be prepared by reacting S26 with methyl 1-bromoacetate and a base, e.g., pyridine. S111 may be prepared by treating S110 with a base (for example 1N NaOH).

In yet another embodiment, the method of the invention further comprises any one of the following steps (e10), (f10), (g10), (h10), (i10), (j10) or (k10):
(e10) treating the compound formed in (d) with BODIPY TMR-X under conditions sufficient to form a compound of the formula:

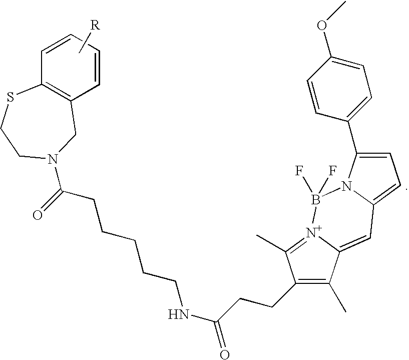

In one embodiment, compound S118 may be prepared from S26 in accordance with step (e10), by treating S26 with BODIPY TMR-X, SE (Molecular Probes Inc.).
(f10) reacting the compound formed in (d) with 4-OH-benzyl bromide or benzyl bromide under conditions sufficient to form a compound of the formula:

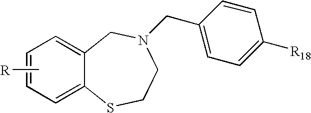

wherein R₁₈ is OH or H.

In one embodiment, the compound S84 is prepared from S68 in accordance with step (f10), by reacting S68 with benzyl bromide.

In another embodiment, the compound S120 may be prepared in an analogous way from S26 in accordance with step (f10), by reacting S26 with 4-OH-benzyl bromide and a base for example Na₂CO₃. In another embodiment, S121 may be prepared by an analogous method to S120, but using benzyl bromide instead of 4-OH-benzyl bromide.
(g10) reacting the compound formed in (d) first with DIEA and then with acetoxyacetyl chloride under conditions sufficient to form a compound of the formula:

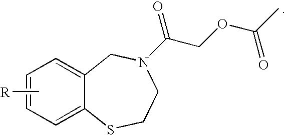

(h10) forming the compound

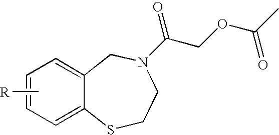

by the method of (g10) and reacting that compound with a base under conditions sufficient to form a compound of the formula:

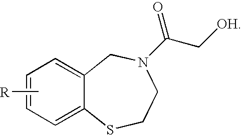

In one embodiment, compounds S122 and S123 may be synthesized in accordance with these steps (g10) and (h10), by reacting S26 with DIEA and subsequently acetoxyacetyl chloride, to form S122. S123 may be prepared by reacting S122 with a base for example LiOH.
(i10) reacting the compound formed in (d) with a compound of formula C₆H₄—NCX₅, wherein X₅ is O or S, under conditions sufficient to form a compound of the formula:

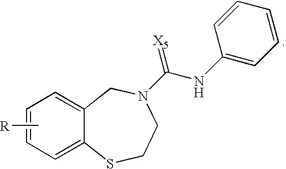

In one embodiment, compounds S11 and S12 may be prepared from S26 in accordance with step (i10), by reacting S26 with a compound of formula C₆H₄—NCX₅, wherein X₅ is O (S11) or S(S12). If necessary, a base catalyst, for example triethylamine or pyridine, is used in the synthesis.
(j10) reacting the compound formed in (d) with phenyl methoxyphosphonyl chloride (Ph(MeO)P(O)Cl) in a solvent to form a reaction mixture, removing the solvent from the reaction mixture to form a residue, and purifying and separating the product into the isomers of compounds of the formulae:

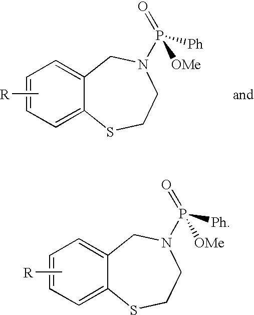

In one embodiment, the isomeric compounds S13 and S14 may be prepared from S26 in accordance with step (j10), by reacting S26 with phenyl methoxyphosphonyl chloride (Ph(MeO)P(O)Cl). If necessary, a base catalyst for example triethylamine may be used.
(k10) reacting the compound formed in (d) with a compound of formula ClOC—X₆—COCl, wherein X₆ is —CH₂—CH₂— or

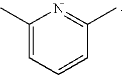

under conditions sufficient to form a compound of the formula:

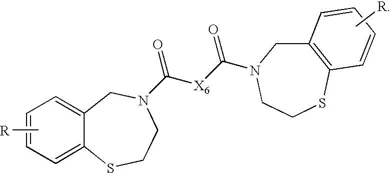

In one embodiment, compounds S19 and S22 may be prepared from S26 in accordance with step (k10), by reacting S26 with a compound of formula ClOC—X₆—COCl, where X₆ is CH₂—CH₂ (S19) or

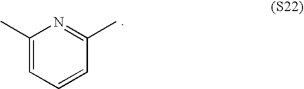

In one embodiment, the invention provides for a method of synthesis of organic compounds from the starting compound

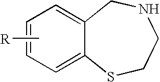

wherein R is H, OR₁, SR₁, N(R₁)₂ alkyl, or halide, and R₁ is independently at each occurrence alkyl, aryl, or H, which comprises any one of the following steps (A-1), (B-1), (C-1), (D-1), (E-1), (F-1), (G-1), (H-1), (I-1), (J-1)(1), (J-1)(2) or (J-1)(3):
(A-1) reacting the starting compound with a compound of formula R₅COX₁, wherein R₅ is —CH₂I, Ph-, CH₂═CH—, 4-N₃-2-OH—C₆H₅ or

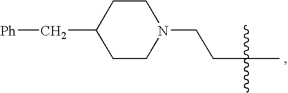

and X₁ is —Cl or —OSu, under conditions sufficient to form a compound of the formula:

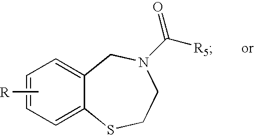

(B-2) forming the compound

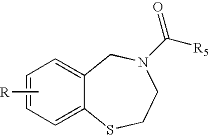

by the method of (A-1) and treating that compound with hydrogen peroxide or m-CPBA, under conditions sufficient to form a compound of the formula:

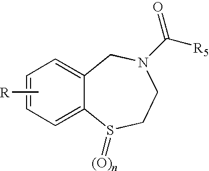

wherein n is 1 or 2; or
(C-1) forming the compound

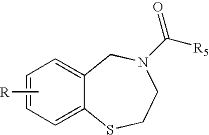

by the method of (A-1), wherein R₅ is CH₂═CH—, and reacting that compound with a compound of formula HNR₈R₉, wherein NR₈R₉ is

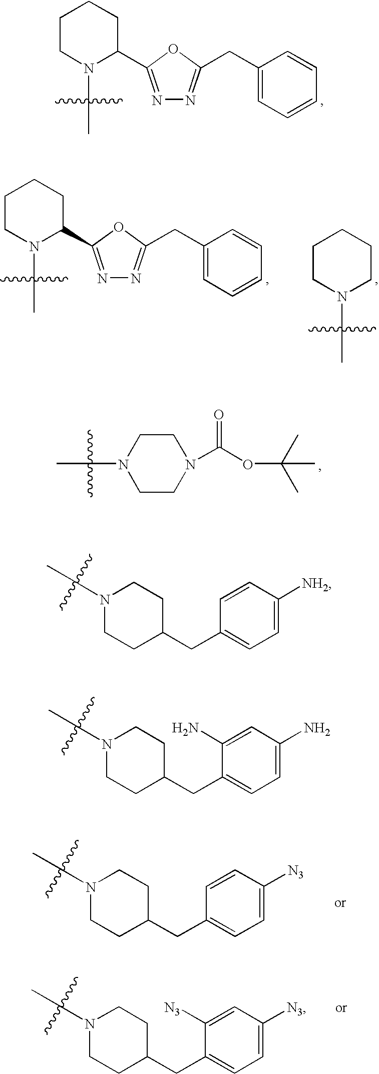

under conditions sufficient to form a compound of the formula:

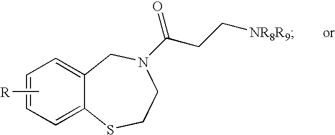

(D-1) reacting the starting compound with an acid chloride of formula Cl—C(═O)C(═O)ORₐₐ, under conditions sufficient to form a compound of the formula:

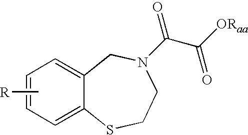

wherein Rₐₐ is C₁-C₄ alkyl or aryl; or
(E-1) forming the compound

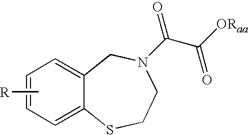

by the method of (D-1) and reacting that compound with an acid or base under conditions sufficient to form a compound of the formula:

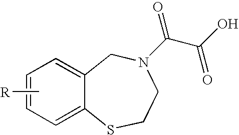

or a salt thereof; or
(F-1) forming the compound

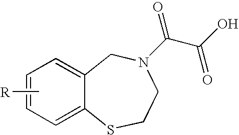

by the method of (E-1) in salt form, and adding an acid to the salt to form a compound of formula

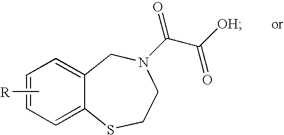

(G-1) forming the compound

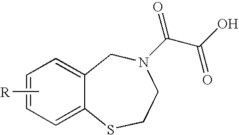

by the method of (E-1) or (F-1) and reacting that compound with hydrogen peroxide under conditions sufficient to form a compound of the formula:

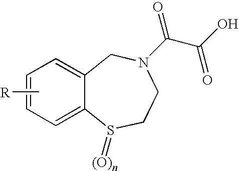

or a salt thereof, wherein n is 1 or 2; or
(H-1) forming the compound

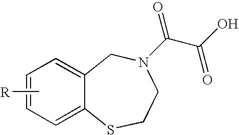

by the method of (E-1) or (F-1), wherein R is methoxy and Rₐₐ is methyl and treating that compound with BBr₃, and reacting that compound under conditions sufficient to form a compound of formula:

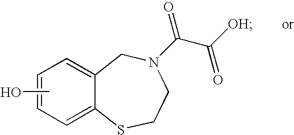

(I-1) forming the compound

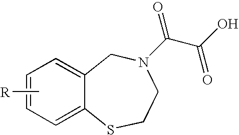

by the method of (E-1) or (F-1), and reacting that compound with thionyl chloride or oxalyl chloride under conditions sufficient to form a compound of the formula:

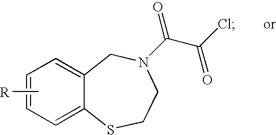

(J-1) forming the compound

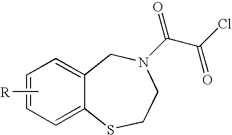

by the method of (I-1) and reacting that compound with:
   (1) cystanine and a base under conditions sufficient to form a compound of the formula:

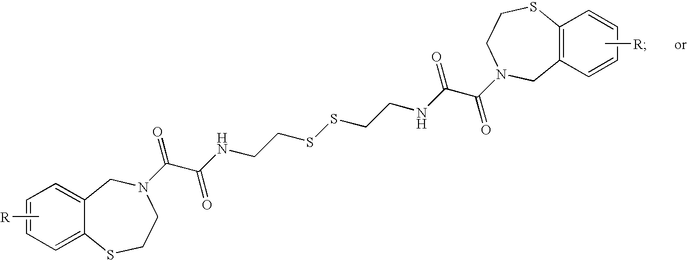

(2) a compound of the formula HX₂ wherein X₂ is —OCH₃ or —NHEt, under conditions sufficient to form a compound of the formula:

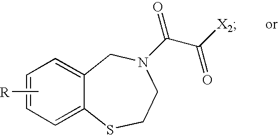

(3) cystamine and a base to form S36-cystamine; and reacting the S36-cystamine with an NHS activated ester of an azido compound under conditions sufficient to form a compound of the formula:

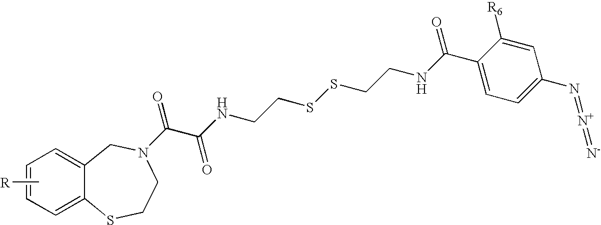

wherein R₆ is —NO₂ or HO—.

In some embodiments, (i) Rₐₐ, is methyl or ethyl; or (ii) the acid in reaction (B-1) or (C-1) is trifluoroacetic acid (TFA) or hydrochloric acid (HCl); or (iii) the base in reaction (B-1) is NaOH, KOH and LiOH; or (iv) R is methoxy; or (v) R is at position 7 of the benzothiazepine ring and the compound has the structure;

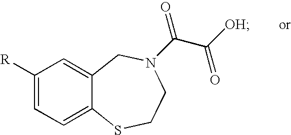

(vi) the compound of formula

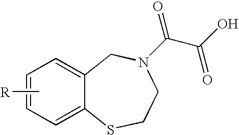

is further purified by dissolving the compound in water to form an aqueous solution; washing the aqueous solution with an organic solvent; acidifying the aqueous solution; and extracting the compound from the aqueous solution using an organic solvent to form an extract; and removing the organic solvent from the extract.

In another embodiment, the invention provides for a method of synthesis of organic compounds from the starting compound

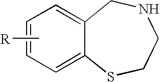

wherein R is H, OR₁, SR₁, N(R₁)₂, alkyl, or halide, and R₁ is independently at each occurrence alkyl, aryl, or H, which comprises any one of the following steps (A-2), (B-2), (C-2), (D-2), (E-2), (F-2), (G-2), (H-2), (1-2), (J-2), (K-2), (L-2), (M-2), (N-2), (O-2), (P-2), (Q-2), (R-2), (S-2), (T-2), (U-2), (V-2), (W-2), (X-2), (Y-2), (Z-2), (AA), (BB) or (CC):
(A-2) reacting the starting compound with a compound of formula R₂SO₂Cl under conditions sufficient to form a compound of the formula:

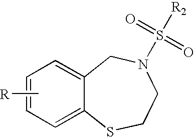

wherein R₂ is CH₂═CH—, Me, p-Me-C₆H₄, or —NH-2-Pyridyl; or
(B-2) forming the compound

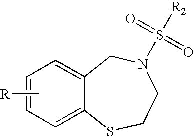

by the method of (A-2), wherein R₂ is CH₂═CH—, and reacting that compound with a compound of formula HNR₃R₄, wherein NR₃R₄ is

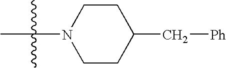

### or —NBu₂,

under conditions sufficient to form a compound of the formula:

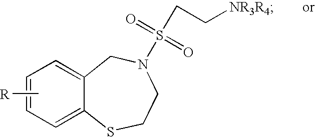

(C-2) reacting the starting compound with SO₂Cl₂ and a base, under conditions sufficient to form a compound of the formula:

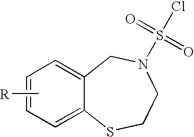

and hydrolyzing that compound to obtain a compound of the formula:

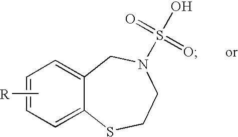

(D-2) reacting the starting compound with
   (1) 4-nitrophenyl chloroformate under conditions sufficient to form a compound of the formula:

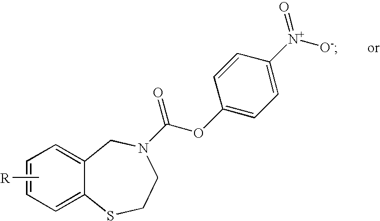

(2) triphosgene and a base to form a compound of the formula

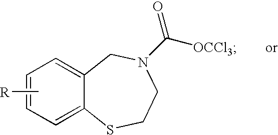

(E-2) reacting the starting compound with
   (1) 4-nitrophenyl chloroformate under conditions sufficient to form a compound of the formula:

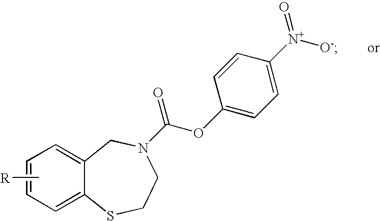

(2) triphosgene and a base to form a compound of the formula

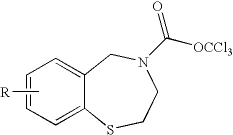

and reacting either compound with an amine of formula HNR₇ₐR_{7b}, wherein NR₇ₐR_{7b} is —NH₂, —NEt₂, —NHCH₂Ph, —NHOH,

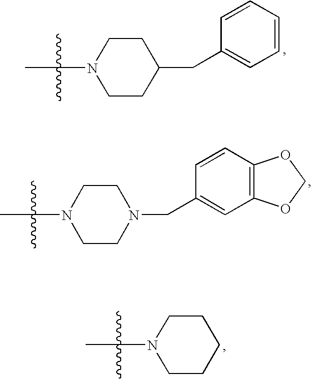

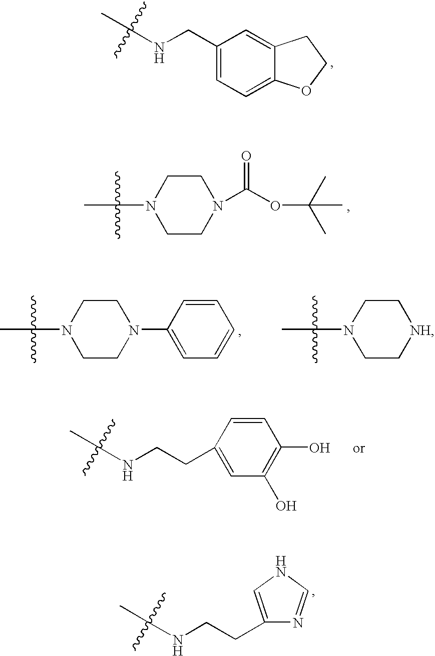

under conditions sufficient to form a compound of the formula:

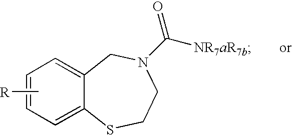

(F-2) reacting an amine of formula HNR₇ₐR_{7b} wherein NR₇ₐR_{7b} is as defined in (E-2) with triphosgene to form a compound of formula Cl₃CO(C═O)NR₇ₐR_{7b}, and reacting that compound with the starting compound to form a compound of the formula:

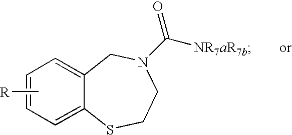

(G-2) forming the compound

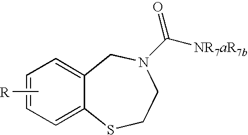

by the method of (E-2) or (F-2), wherein NR₇ₐR_{7b} is

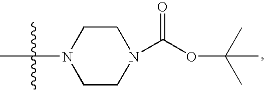

and reacting that compound with a Lawesson's Reagent under conditions sufficient to form a compound of the formula

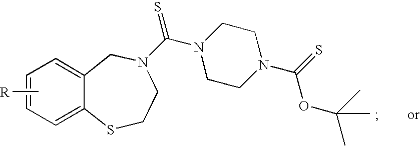

(H-2) forming the compound

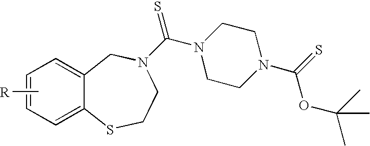

by the method of (G-2) and reacting that compound with an acid under conditions sufficient to form a compound of the formula:

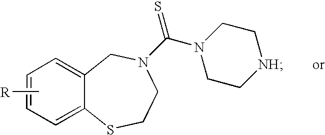

(I-2) reacting the starting compound, wherein R is at position 7 of the benzothiazepine ring, with Boc₂O or Boc-Cl under conditions sufficient to form a compound of the formula:

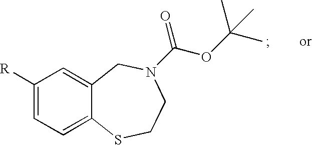

(J-2) forming the compound

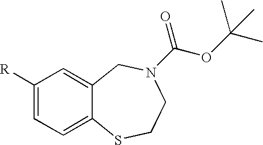

by the method of (I-2) and reacting that compound, wherein R is methoxy, with BBr₃ under conditions sufficient to form a compound of the formula:

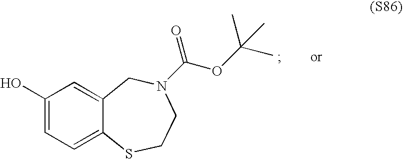

(K-2) forming the compound S86 by the method of (J-2) and reacting that compound with trifluoromethylsulfonyl anhydride, under conditions sufficient to form the compound S87

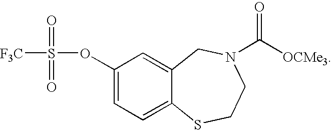

(L-2) forming the compound S87 by the method of (K-2) and reacting that compound with tris(dibenzylideneacetone)dipalladium(0), 2-(di-tert-butylphosphino)-bipihenyl, and K₂PO₃, under conditions sufficient to form compound S88

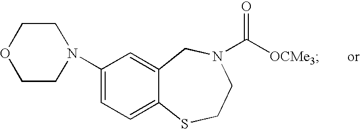

(M-2) forming the compound S87 by the method of (K-2) and reacting that compound with benzenethiol, optionally with a catalyst, under conditions sufficient to form compound S89

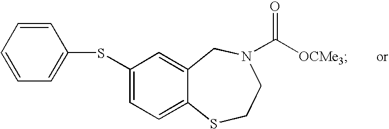

(N-2) forming the compound S87 by the method of (K-2) and reacting that compound with a base, phenylboronic acid, and a catalyst, under conditions sufficient to form compound S90

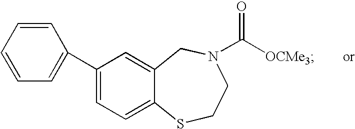

(O-2) forming the compound S87 by the method of (K-2) and reacting that compound with propene in the presence of a metal catalyst, a ligand and a base, under conditions suitable to form the compound S91

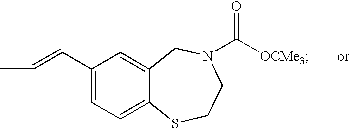

(P-2) forming the compound S87 by the method of (K-2) and reacting that compound with zinc cyanide and a catalyst, under conditions sufficient to form compound S92

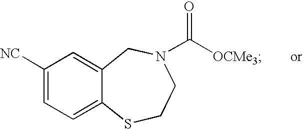

(Q-2) forming the compound S87 by the method of (K-2) and reacting that compound with benzylamine in the presence of a catalyst, a ligand and a base, under conditions suitable to form the compound S93

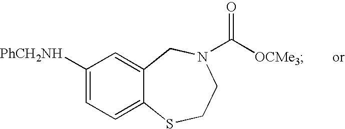

(R-2) forming the compound S86 by the method of (J-2) and reacting that compound with acetic anhydride, under conditions sufficient to form compound S94

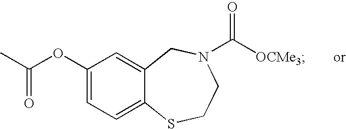

(S-2) forming the compound S94 by the method of (R-2), reacting that compound with AlCl₃, and reattaching the Boc group if necessary, under conditions sufficient to form compound S95

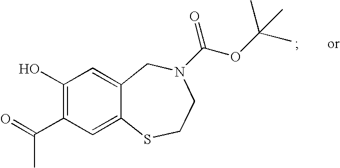

(T-2) forming the compound S86 by the method of (J-2) and reacting that compound with NaI and Chloramine-T, under conditions sufficient to form compound S96

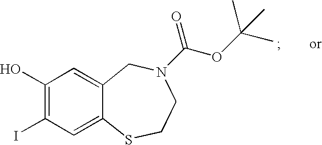

(U-2) forming the compound S86 by the method of (J-2) and reacting that compound with H₂SO₄ to form a sulfuric acid mixture; then adding HNO₃ to the sulfuric acid mixture thus formed, and reattaching the Boc group, under conditions sufficient to form compound S97

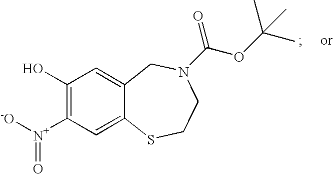

(V-2) forming the compound S97 by the method of (U-2) and hydrogenating that compound under conditions sufficient to form compound S98

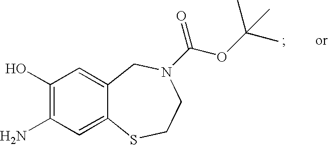

(W-2) forming the compound S98 by the method of (V-2) and reacting that compound with sodium nitrite and NaBaF₄ under conditions sufficient to form compound S99

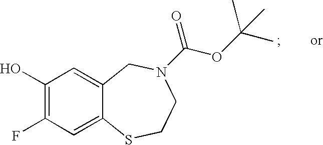

(X-2) forming the compound S98 by the method of (V-2), dissolving that compound in an acidic solution to form a mixture and then reacting the mixture with sodium nitrite and NaN₃, and reattaching the Boc group, under conditions sufficient to form compound S100

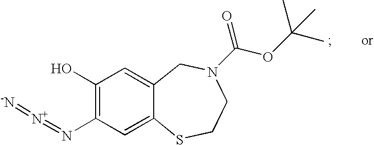

(Y-2) reacting the starting compound with a compound of formula to produce a compound of formula:

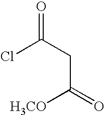

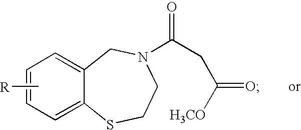

(Z-2) forming the compound

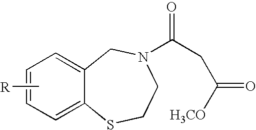

by the method of (Y-2) and reacting that compound with an acid or base under conditions sufficient to form a compound of the formula:

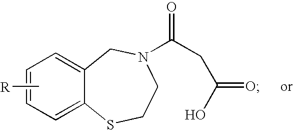

(AA) reacting the starting compound with formaldehyde (CH₂O) and a reducing agent, under conditions sufficient to form a compound of the formula:

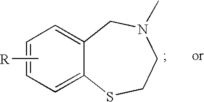

(BB) forming the compound

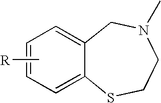

by the method of (AA) and reacting that compound with CH₃X₃, under conditions sufficient to form a compound of the formula:

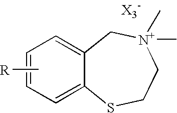

wherein X₃ is a halogen selected from F, Cl, Br and I; or
(CC) forming the compound

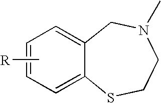

by the method of (AA) and reacting that compound with hydrogen peroxide, under conditions sufficient to form a compound of the formula:

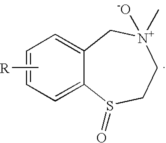

In some embodiments (i) R is methoxy and is located at position 7 of the benzothiazepine ring or (ii) the reducing agent in (A-2) is sodium cyanoborohydride (NaBCNH₃) or sodium triacetoxyborohydride.

In another embodiment, the invention provides for a method of synthesis of organic compounds from the starting compound

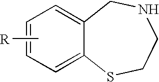

wherein R is H, OR₁, SR₁, N(R₁)₂, alkyl, or halide, and R₁ is independently at each occurrence alkyl, aryl, or H, which comprises any one of the following steps (A-3), (B-3), (C-3), (D-3), (E-3), (F-3), (G-3), (1-3), (I-3), (J-3) or (K-3):
(A-3) reacting the starting compound with N-benzyloxycarbonyl-glycine (Cbz-Gly), under conditions sufficient to form a compound of the formula:

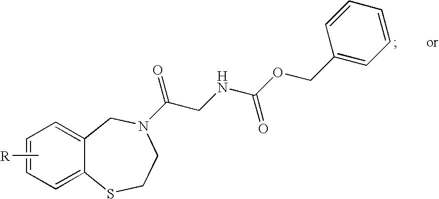

(B-3) forming the compound

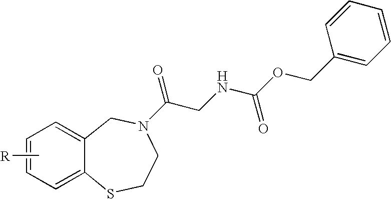

by the method of (A-3) and treating that compound with an acid under conditions sufficient to form a compound of the formula:

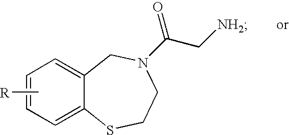

(C-3) reacting the starting compound with a compound of formula:

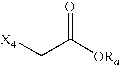

wherein X₄ is a halogen or a sulfonate, and Rₐ is a C₁-C₄ alkyl, under conditions sufficient to form a compound of the formula:

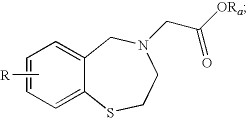

(D-3) forming the compound′

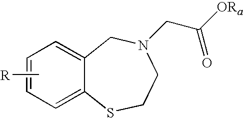

by the method of (C-3) and treating that compound with an acid or a base under conditions sufficient to form a compound of the formula:

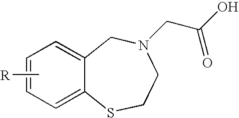

or a salt thereof; or
(E-3) reacting the starting compound with BODIPY TMR-X under conditions sufficient to form a compound of the formula:

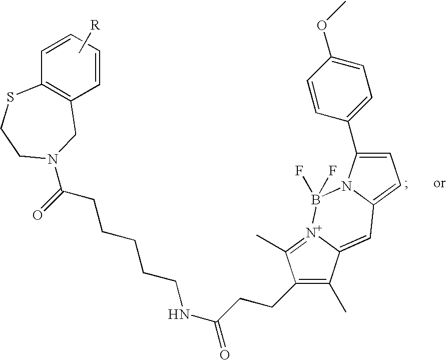

(F-3) reacting the starting compound with 4-OH-benzyl bromide or benzyl bromide under conditions sufficient to form a compound of the formula:

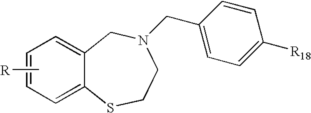

wherein R₁₈ is OH or H; or
(G-3) reacting the starting compound with DIEA and then with acetoxyacetyl chloride, under conditions sufficient to form a compound of the formula:

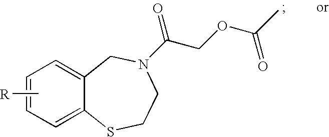

(H-3) forming the compound

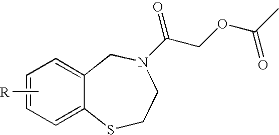

by the method of (G-3) and reacting that compound under conditions sufficient to form a compound of the formula:

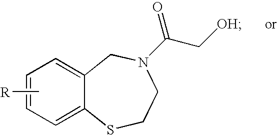

(I-3) reacting the starting compound with a compound of formula C₆H₄—NCX₅, wherein X₅ is O or S, under conditions sufficient to form a compound of the formula:

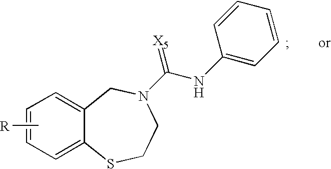

(J-3) reacting the starting compound with phenyl methoxyphosphonyl chloride (Ph(MeO)P(O)Cl) in a solvent to form a reaction mixture; removing the solvent from the reaction mixture to form a residue; and purifying and separating the product into the isomers of compounds of the formulae:

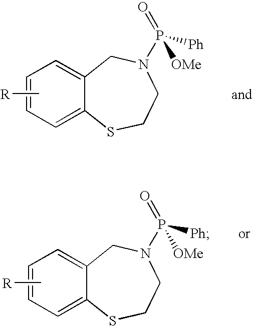

(K-3) reacting the starting compound with a compound of formula ClOC—X₆—COCl, wherein X₆ is —CH₂—CH₂— or

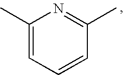

under conditions sufficient to form a compound of the formula:

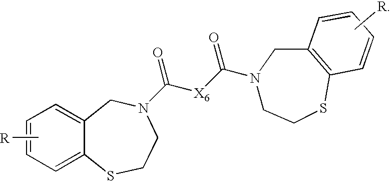

The present invention encompasses the synthesis of any of the starting materials or intermediates disclosed herein.

Some of the syntheses described herein utilize solvents. In one embodiment, the solvent is an organic solvent. In another embodiment, the organic solvent is methylene chloride (CH₂ Cl₂), chloroform (CCl₄), tetrahydrofuran (THF), methanol (CH₃OH), acetonitrile, ethyl acetate, dimethylfoimamide (DMF), diethyl ether, dioxane or pyridine, or a mixture thereof.

Some of the syntheses described herein also utilize a base reagent. In one embodiment, the base reagent is an amine compound. In another embodiment, the base reagent is an alkylamine for example triethylamine (TEA). In still another embodiment, the base reagent is pyridine. In still embodiments, the base reagent is a carbonate, for example sodium, potassium and cesium carbonate: sodium hydride: diisopropylamine (DIPEA): N-methylmorpholine (NMM); or the like.

Some of the syntheses described herein also utilize basic solutions. In one embodiment, the basic solution is sodium bicarbonate or calcium carbonate. In another embodiment, the basic solution is saturated sodium bicarbonate or saturated calcium carbonate.

Some of the syntheses described herein use acidic solutions. In one embodiment, the acidic solution is a sulfuric acid solution, a hydrochloric acid (HCl) solution, a hydrobromic acid (HBr) solution, or a nitric acid solution. In one embodiment, the solution is 1N HCl. In another embodiment, the solution is a hydrochloric acid solution in an organic acid for example diethyl ether or dioxane, or a hydrogen bromide solution in a solvent for example acetic acid. In another embodiment, the acidic solution is a trifluoroacetic acid (TFA) solution, which may be either neat, or in an organic solvent. One of skill in the art will appreciate still other solvents, organic solvents, base catalysts, basic solutions, and acidic solutions can be used in the embodiments according to the description herein. The solvents, organic solvents, reactants, catalysts, wash solutions, and so forth are added at appropriate temperatures (e.g. room temperature or about 20° C.-25° C., 0° C., etc.).

Some of the syntheses described herein require purification of the reaction mixture to yield a final product. Purification of the reaction mixture involves one or more processes for example removal of any solvent, crystallization of the product, distillation of the product, chromatographic separation of the product (including HPLC, silica gel chromatography, column chromatography, and so forth), washing with basic solution, washing with acidic solution, re-dissolving the product in another solvent, and so forth. One of skill in the art will appreciate still other processes can be used in the embodiments according to the description herein.

The reactions are carried out as long as needed (e.g., one hour, several hours, overnight, 24 hours, etc.). Often, the reaction mixtures are stirred. The reactions are carried out at appropriate temperatures (e.g. room temperature or about 20° C.-25° C., 0° C., 100° C., etc.).

### DEFINITIONS

Alkyl is intended to include linear, branched, or cyclic hydrocarbon structures and combinations thereof. For example, alkyl refers to alkyl groups of from 1 to 6 carbon atoms. Examples of alkyl groups include methyl, ethyl, propyl, isopropyl, butyl, s- and t-butyl and the like. Alkyl groups can be those of C₂₀ or below. Other examples of alkyls include C₁-C₄ alkyl. C₁-C₈ alkyls, C₁-C₁₀ alkyls and C₁-C₁₂ alkyls Cycloalkyl is a subset of alkyl and includes cyclic hydrocarbon groups of from 3 to 8 carbon atoms. Examples of cycloalkyl groups include cyclopropyl, cyclobutyl, cyclopentyl, norbornyl and the like. The term “heteroalkyl” means an alkyl which contains one or more heteroatoms, for example N, O, or S.

The substituent “OR” designates an alkoxy or alkoxyl and refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. For example, and alkyloxy can refer to groups containing one to four carbons. Methoxy is an example. For the purpose of this application, alkoxy and lower alkoxy include methylenedioxy and ethylenedioxy.

The substituent “SR” designates a thioalkyl and refers to groups of from 1 to 8 carbon atoms of a straight, branched, cyclic configuration and combinations thereof attached to the parent structure through a sulfur. Examples include thiomethyl, thioethyl and the like. Lower-thioalkyl refers to groups containing one to four carbons.

Aryl (Ar) refers to a 5- or 6-membered aromatic or heteroaromatic ring containing 0-3 heteroatoms selected from O, N, or S; a bicyclic 9- or 10-membered aromatic or heteroaromatic ring system containing 0-3 heteroatoms selected from O, N, or S; or a tricyclic 13- or 14-membered aromatic or heteroaromatic ring system containing 0-3 heteroatoms selected from O, N, or S. The aromatic 6- to 14-membered carbocyclic rings include, e.g., benzene, naphthalene, indane, tetralin, and fluorene and the 5- to 10-membered aromatic heterocyclic rings include, e.g., imidazole, pyridine, indole, thiophene, benzopyranone, thiazole, furan, benzofuran, benzimidazole, quinoline, isoquinoline, quinoxaline, pyrimidine, pyrazine, tetrazole and pyrazole. The aryl may be linked to the molecule through an alkyl (i.e., an arylalkyl for example benzyl, phenethyl, pyridinylmethyl, pyrimidinylethyl and the like).

Heterocycle means a cycloalkyl or aryl residue in which from one to three carbons is replaced by a heteroatom selected from the group consisting of N, O and S. The nitrogen and sulfur heteroatoms may optionally be oxidized, and the nitrogen heteroatom may optionally be quaternized. Examples of heterocycles include, but are not limited to, pyrrolidine, pyrazole, pyrrole, tetrahydroisoquinoline, benzodioxan, benzodioxole (commonly referred to as methylenedioxyphenyl, when occurring as a substituent), tetrazole, morpholine, thiazole, pyridine, pyridazine, piperidine, piperazine, pyrimidine thiophene, furan, oxazole, oxazoline, isoxazole, dioxane, tetrahydrofuran and the like. It is to be noted that heteroaryl is a subset of heterocycle in which the heterocycle is aromatic.

Substituted alkyl, aryl, cycloalkyl, heteroalkyl, heterocycle, heteroaryl, etc. refer to alkyl, aryl, cycloalkyl, heteroalkyl, heterocyclyl, or heteroaryl, etc. wherein up to three H atoms in each residue are replaced with halogen, haloalkyl, hydroxy, alkoxy, carboxy, carboalkoxy (also referred to as alkoxycarbonyl), carboxamido (also referred to as alkylaminocarbonyl), cyano, carbonyl, nitro, amino, alkylamino, dialkylamino, mercapto, alkylthio, sulfoxide, sulfone, acylamino, amidino, phenyl, benzyl, heteroaryl, phenoxy, benzyloxy, or heteroaryloxy.

The term “halogen” or “halide” means fluorine, chlorine, bromine or iodine. For example, the term “chloride” refers to a Cl atom. For example, a carboxylic acid may be reacted with a chloride such that the hydroxyl group of the acid is replaced by a Cl (i.e., the conversion of RCO₂H to RCOCl.

An amine refers to an N(R)₂ group where each R may independently be hydrogen, an alkyl or aryl as defined herein.

The term disulfide refers to a “—S—S—” group.

A basic solution is a solution of an inorganic or organic base. An acidic solution is a solution of an inorganic or organic acid.

The term about means +/−20% of the indicated valued. For example, the term “100” encompasses 99.5, 99, 98, 95, 90 or 80, etc.

The present invention further provides methods of synthesizing radio-labeled 1,4-benzothiazepine compounds and derivatives. Labeling of the compounds is accomplished by using one of a variety of different radioactive labels known in the art. The radioactive label of the present invention is, for example, a radioisotope. The radioisotope is any isotope that emits detectable radiation including, without limitation, ³⁵S, ¹²⁵I, ³H, or ¹⁴C. Radioactivity emitted by the radioisotope may be detected by techniques well known in the art. For example, gamma emission from the radioisotope is detected using gamma imaging techniques, particularly scintigraphic imaging. Radiolabeled compounds can be synthesized by replacing a reactant with a radiolabeled version of that reactant

By way of example, radio-labeled compounds are prepared as follows. A compound of the invention may be demethylated at the phenyl ring using BBr₃. The resulting phenol compound then is re-methylated with a radio-labeled methylating agent (for example ³H-dimethyl sulfate) in the presence of a base (for example NaH) to provide ³H-labeled compounds.

The present invention is described in the following Examples, which are set forth to aid in the understanding of the invention and should not be construed to limit in any way the scope of the invention as defined in the claims which follow thereafter.

### PROPHETIC EXAMPLES

The examples provided herein are illustrative in nature and in no way restrict the broad scope of the present invention. In addition, the particular conditions of each exemplary reaction may be determined by a person of skill in the art.

Initial attempts to prepare 7-methoxy-2,3,4,5-tetrahydro-1,4-benzothiazepine (S26) as previously reported, were unsuccessful. The thio group of the intermediate is easily oxidized by air to a disulfide compound, which makes the synthesis of a cyclized product impossible. To overcome this problem, the inventors developed a novel process that starts with the readily-available and inexpensive 2-nitro-5-methoxybenzoic acid.

Synthesis of S26 (Scheme 1, compound (I) when R═OMe): Reduction of the nitro group of 2-nitro-5-methoxybenzoic acid (16), using H, with Pd/C as a catalyst, gives 2-amino-5-methoxybenzoic acid (17) in quantitative yield. Compound (17) is diazotized with NaNO₂, and then treated with Na₂S₂ to provide the stable disulfide compound (18) with 80% yield. Without further purification, the stable disulfide (18) is treated with SOCl₂, and then reacted with 2-chloroethylamine, in the presence of Et₃N, to give an amide (19) in 90% yield. Compound (19) is converted to cyclized compound (10) (S25 when R═OMe) via a one-pot procedure by reflux with trimethylphosphine and Et₃N in THF. Alternatively, dithionite may be used instead of trimethylphosphine, and a carbonate, for example sodium carbonate, may be used instead of Et₃N. The cyclized amide (10) is then reduced with LiAlH₄ to yield 7-methoxy-2,3,4,5-tetrahydro-1,4-benzothiazepine (S26).

Synthesis of JTV-519 (Scheme 1): JTV-519 is prepared by reacting compound (1) (S26) with 3-bromopropionic chloride, and then reacting the resulting product with 4-benzyl piperidine. The structure of JTV-519 is established by ¹H NMR.

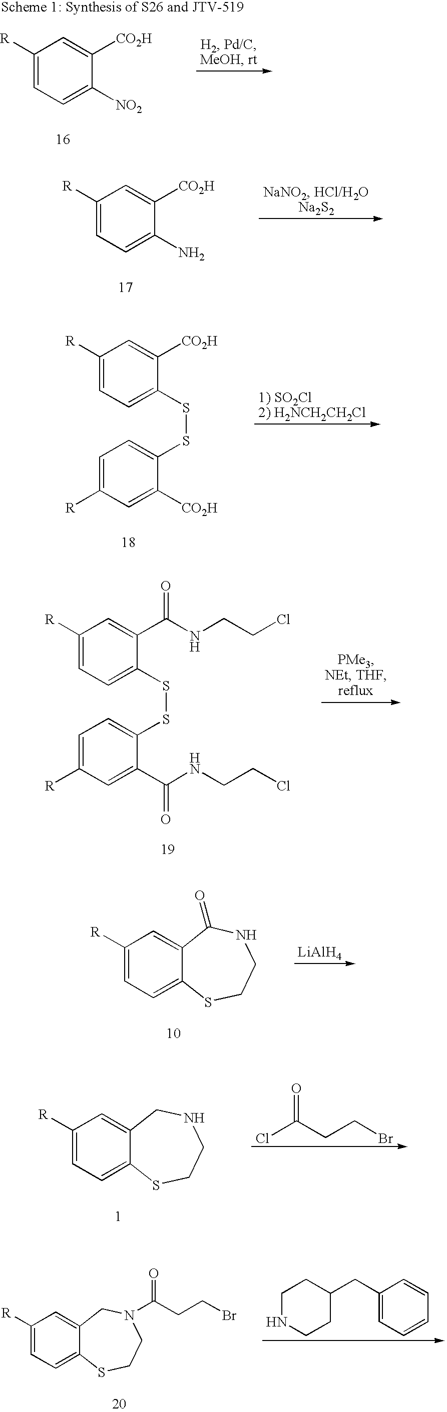

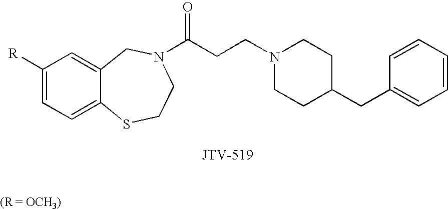

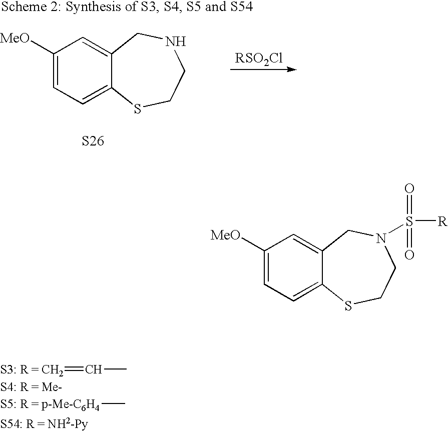

In scheme 2, NH 2-Py refers to NH-2-pyridine

Synthesis of S3 (Scheme 2): To a stirred solution of vinylsulfonic acid (22 mg, 0.2 mmol) in anhydrous CH₂Cl₂ (5 ml) is added thionyl chloride (2M in CH₂Cl₂, 0.1 ml, 0.2 mmol). The reaction mixture is stirred at room temperature overnight and evaporated under vacuum. The residue is dissolved in CH₂Cl₂ (5 ml). To this solution, a solution of S26 (20 mg, 0.1 mmol) in CH₂Cl₂ (3 ml) is added drop-wise at 0° C. The reaction mixture is stirred at 0° C. for one hour and at room temperature for another hour and washed with saturated sodium bicarbonate and 1N HCl. After removal of the solvent, the product S3 is purified by SiO₂ column chromatography as a colorless oil (18 mg, 65%).

Synthesis of S4 (Scheme 2): To a stirred solution of S26 (20 mg, 0.1 mmol) in CH₂Cl₂ (5 ml) is added methylsulfonyl chloride (26 mg, 0.2 mmol) and triethylamine (30 mg, 0.3 mmol) at 0° C. The resulting mixture is stirred at 0° C. for one hour and at room temperature overnight. The organic phase is washed with aqueous saturated sodium bicarbonate and dried over sodium sulfate. After filtration and evaporation of the organic solvents, the product S4 is purified by SiO₂ column chromatography (25 mg oil, yield: 90%).

Similarly, S5 and S54 are synthesized from p-Me-C₆H₄ sulfonyl chloride and NH-2-pyr-sulofnyl chloride, respectively, in 95% and 91% yields, respectively.

Synthesis of S1 and S2 (Scheme 3): To a solution of S3 (28 mg, 0.1 mmol) in chloroform (5 ml) is added 4-benzylpiperidine (18 mg, 0.1 mmol). The resulting mixture is stirred at room temperature for 1 day. After removal of organic solvent, the residue is purified on silica gel column. Product S1 is obtained as a colorless oil (34 mg, yield: 75%). S2 is synthesized similarly from S3 and dibutylamine in 78% yield.

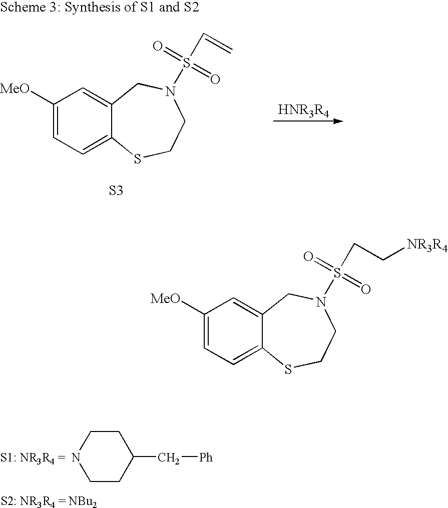

Synthesis of S7, S9, S27 and S40 (Scheme 4): To a stirred solution of iodoacetic acid (37 mg, 0.2 mmol) in CH₂Cl₂ (10 ml) is added thionyl chloride (2 M solution in CH₂Cl₂, 0.1 ml, 0.2 mmol). The resulting mixture is stirred at 0° C. for one hour and at room temperature overnight. After removal of solvent, the crude acid chloride is added to a stirred solution of S26 (20 mg, 0.1 mmol) and triethylamine (30 mg, 0.3 mmol) in CH₂Cl₂ (10 ml) at 0° C. The mixture is stirred at 0° C. for one hour and at room temperature overnight. The organic phase is washed with saturated sodium bicarbonate and 1N HCl. The crude product is purified by column chromatography to give S7 as a colorless oil (34 mg, yield: 95%). Similarly, S9 is synthesized in 95% yield using Ph-COOH as a starting material; S27 is synthesized in 96% yield using CH₂═CH—CO₂H as a starting material; and S40 is synthesized in 91% yield using N-hydroxysuccinimidyl 4-azidosalicylic acid (NHS-ASA) as the starting material.

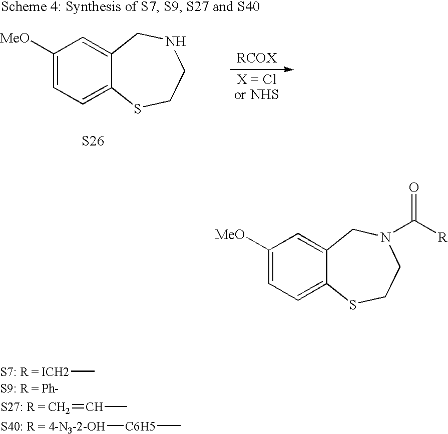

Synthesis of S11 and S12 (Scheme 5): To a solution of S26 (20 mg, 0.1 mmol) in pyridine (1 ml) is added phenyl isocyanate (18 mg, 0.15 mmol). The resulting mixture is stirred at room temperature for 24 hours. Then ethyl acetate (10 ml) is added and the organic phase is washed with 1N HCl and saturated sodium bicarbonate. The product S11 is purified by SiO₂ column chromatography as a white solid (27 mg, yield: 86%). Similarly S12 is synthesized from S26 and phenyl isothiocyanate in 85% yield.

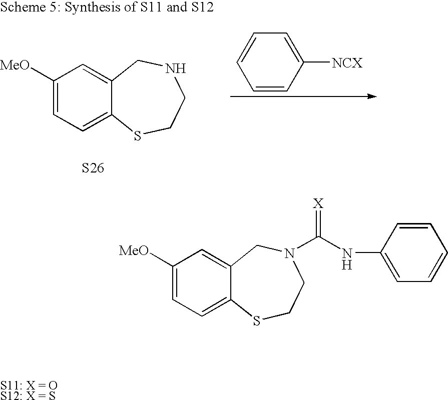

Synthesis of S13 and S14 (Scheme 6): To S26 (20 mg, 0.1 mmol) in CH₂Cl₂ (5 ml) is added triethylamine (30 mg, 0.3 mmol) and phenyl methoxyphosphlonyl chloride (38 mg, 0.2 mmol) at 0° C. After stirring for 2 hours at room temperature, the reaction mixture is washed with saturated sodium bicarbonate. Isomers are separated and purified by silica gel column to yield S13 (14 mg, yield: 40%) and S14 (16 mg, yield: 45%).

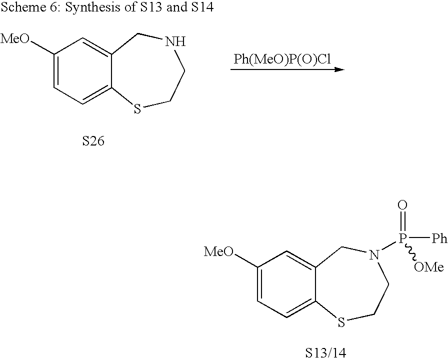

Synthesis of S19 (Scheme 7): To a stirred solution of S26 (20 mg, 0.1 mmol) and triethylamine (30 mg, 0.3 mmol) in CH₂Cl₂ (5 ml) is added 1,4-butyldiacid chloride (8 mg, 0.05 mmol) at 0° C. The resulting mixture is stirred at 0° C. for one hour and at room temperature overnight. The organic phase is washed with saturated sodium bicarbonate and 1N HCl and water. After removal of solvent, product S19 is purified by column chromatography (oil, 19 mg, 80% yield). Similarly, S22 is prepared from 2,6 pyridyl dicarboxylic acid dichloride.

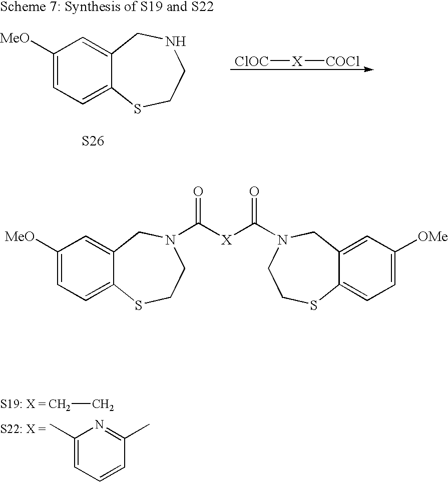

Synthesis of S20 and S23 (Scheme 8): S27 (25 mg, 0.1 mmol) in MeOH (5 ml) is treated with H₂O₂ (30%, 0.5 ml) at room temperature for 1 day. After treatment with sodium thiosulfate solution, methanol is removed by evaporation. The resulting residue is dissolved in ethyl acetate (10 ml) and washed with saturated sodium carbonate. After drying over sodium sulfate, solvent is evaporated to provide a crude product which is purified by silica gel column chromatography to yield S20 as colorless oil (16 mg, 60% yield). Similarly, S23 is synthesized from S10 (JTV519).

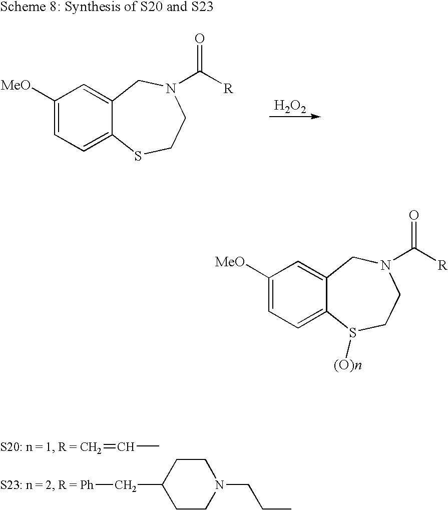

Synthesis of S36 and S57 (Scheme 9A): To a stirred solution of S26 (0.85 g, 4.4 mmol) and pyridine (0.70 g, 8.8 mmol) in CH₂Cl₂ (50 ml) at 0° C. is added drop-wise methyl chlorooxoacetate (0.81 g, 6.6 mmol). The reaction mixture is stirred at 0° C. for 2 hours then washed with saturated sodium bicarbonate, 1N HCl, and water. Silica gel column chromatography provides S57 as a white solid (1.1 g, 90% yield). S57 (1.1 g, 3.9 mmol) is dissolved in methanol (10 ml) and then a solution of sodium hydroxide (0.3 g, 7.5 mmol) in water (10 ml) is added. The reaction mixture is stirred at room temperature for one hour. After solvent is removed, the residue is dissolved in water (10 ml) and washed with ether (2×10 ml). The aqueous phase is acidified with 1N HCl to pH=2. The product is extracted with CH₂Cl₂ (2×10 ml). Removal of solvent yields product S36 as a white solid (1.0 g, yield 100%). The product may be further purified by recrystallization.

Synthesis of S43, S44, S45 and S59 (Scheme 9A): S36 (150 mg, 0.56 mmol) is treated with thionyl chloride (5 ml) at room temperature overnight. After removal of the excess thionyl chloride, the crude product S36-Cl is dissolved in CHCl₂ (10 ml) and, to this solution, mono-Boc protected cystamine and pyridine (0.2 ml, 196 mg, 2.48 mmol) are added at 0° C. The reaction mixture is stirred at 0° C. for one hour and at room temperature overnight and quenched with saturated sodium bicarbonate. The organic phase is separated and the solvent is removed to give intermediate mono-Boc protected S36-cystamine, which is purified by SiO₂ column cluomatography in 80% yield. Deprotection of the Boc-group is achieved with trifluoroacetic acid in CH₉Cl₉, and the deprotected S36-cystamine is used for the synthesis of S43 and S45 by reaction with NHS-activated esters of azido compounds. Yield is 75% for S43 and 80% for S45.

S44 is synthesized by the following reaction: S36 (50 mg, 0.19 mmol) is treated with thionyl chloride (2 ml) at room temperature overnight. After removal of the excess thionyl chloride, the crude product is dissolved in CH₂Cl₂ (5 ml). To this solution, cystamine (134 mg, 0.88 mmol) and pyridine (98 mg, 1.23 mmol) in CH₂Cl₂ (10 ml) are added and the reaction mixture is stirred at room temperature overnight. S44 is purified by column as a white solid (20 mg, 16% yield). Similarly, S57 and S59 are synthesized by reaction of S36-Cl with methanol or ethylamine (Scheme 9A).

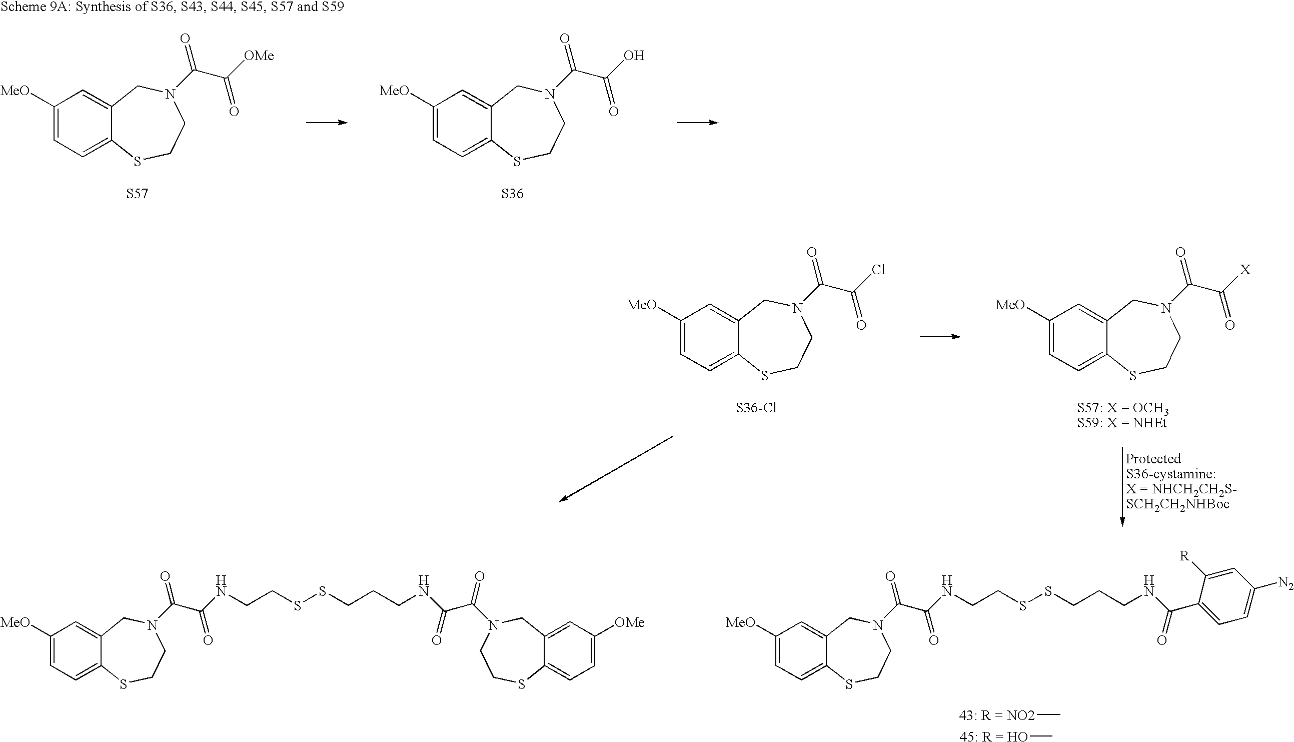

By analogy to the syntheses of S36, S43-S45, S57, and S59 (Scheme 9A), S76-S81 are synthesized from S68 as shown in Scheme 9B in 70-95% yield.

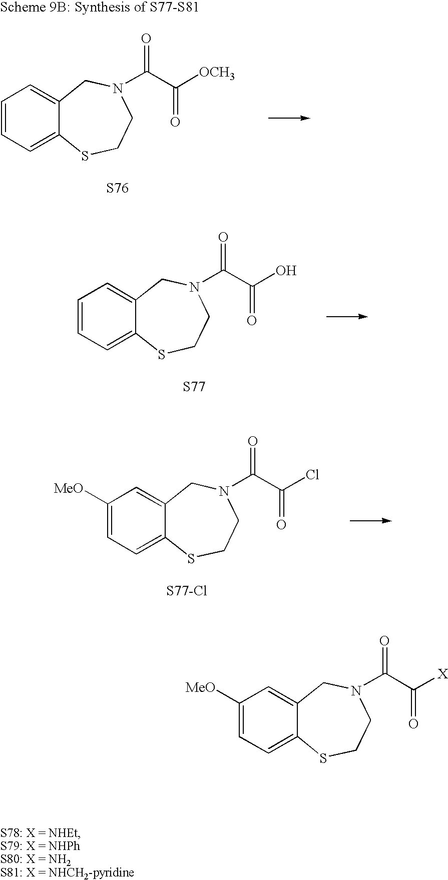

Using S68 as starting material, the compounds S82, S83, and S84 are synthesized as shown in Scheme 9C.

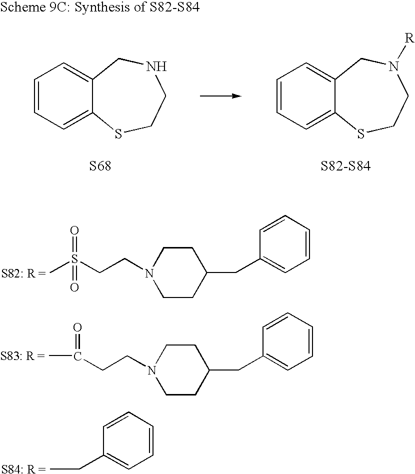

Synthesis of urea-based analogs S6, S46-S53, S64, S66, S67 (Scheme 10A). To S26 (195 mg, 1.0 mmol) in CH₂Cl₂ (20 ml) is added 4-nitrophenyl chloroformate (220 mg, 1.1 mmol) and triethylamine (120 mg, 1.2 mmol) at 0° C. The reaction mixture is stirred for 2 hours at room temperature and washed with water. Removal of the solvents, followed by purification using column chromatography provides compound S37 (330 mg, 91% yield). Reaction of S37 (36 mg, 0.1 mmole) with one equivalent of amine in DMF (3 ml) overnight provides urea-based compounds in >60% yield after purification by SiO₂ column chromatography. Alternatively, the urea-based compounds may be synthesized through a versatile and more reactive intermediate S26-phosgene shown in Scheme 10A.

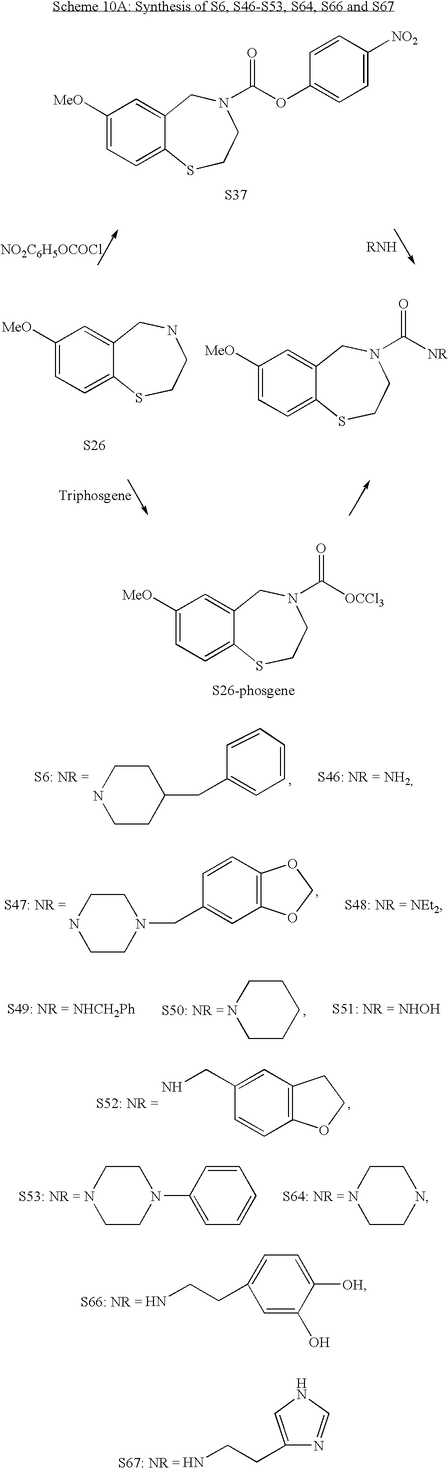

Analogous compounds S69-S75, having no methoxyl groups on the benzene ring (R═H), are synthesized as shown in Scheme 10B in a similar manner to that employed in the synthesis of S46-S53 (Scheme 10A). The synthesis starts with the known S68 and involves a versatile intermediate, S68-phosgene, to provide S69-S75 in 60-95% yield. The preparation of S68 is described, for example, in WO01/55118.

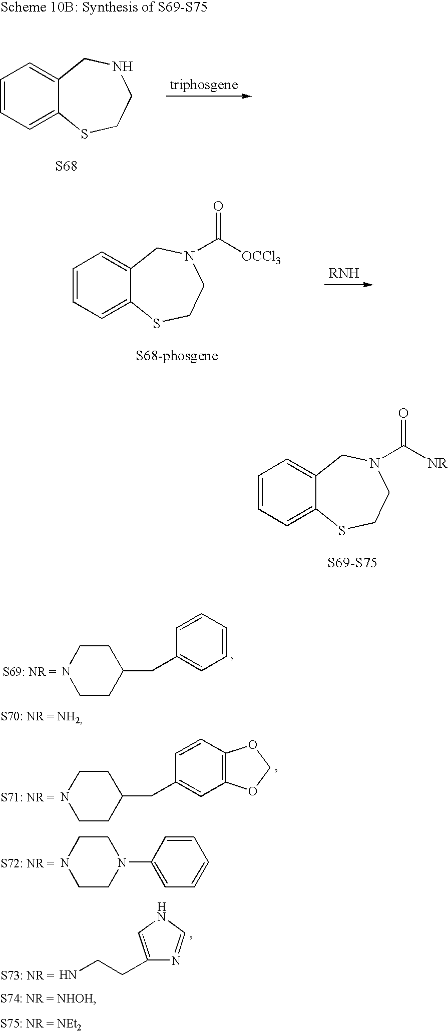

Analogous compounds S101, S102 and S103 are synthesized as shown in Scheme 10C in a similar manner to that employed in the synthesis of S46-S53 (Scheme 10A), and S69-75 (Schemd 10A). The following are examples of the synthesis.

Synthesis of S101: A solution of S68 (165 mg. 1 mmol) in CH₂Cl₂ (50 ml) is cooled to 0° C. To this solution triphosgene (150 mg. 0.5 mmol) and pyridine (0.5 ml. excess) are added and the reaction is stirred at 0° C. for 1 hour. Without purification, the resulting S68-phosgene in the reaction mixture is treated with 1-(benzo[d][1,3]dioxol-5-ylmethyl)piperazine (233 mg, 1.1 mmol) at 0° C. After stirring at 0° C. for 1 hour, the reaction mixture is washed with H₂O (2×10 ml), 1N HCl (2×10 ml) and saturated NaHCO₃ (2×10 ml), and the solvents are removed under reduced pressure. Purification by SiO₂ column chromatography provides ARM101 (i.e. S101) having a yield of 80%. The structure of the product is confirmed by nuclear magnetic resonance (NMR), mass spectroscopy (MS) and/or by elemental analysis.

Synthesis of S102: S102 is synthesized from S68 using the same method used to synthesize S101, with the exception that piperidine is used in place of 1-(benzo[d][1,3]dioxol-5-ylmethyl)piperazine. The structure of the product is confirmed by nuclear magnetic resonance (NMR), mass spectroscopy (MS) and/or by elemental analysis.

Synthesis of S103: S103 is synthesized from S68 using the same method used to synthesize S101, with the exception that N-Boc-1-piperazine is used in place of 1-piperonylpiperazine, and in a subsequent step the Boc group is deprotected using trifluoroacetic acid (TFA). The structure of the product is confirmed by nuclear magnetic resonance (NMR), mass spectroscopy (MS) and/or by elemental analysis.

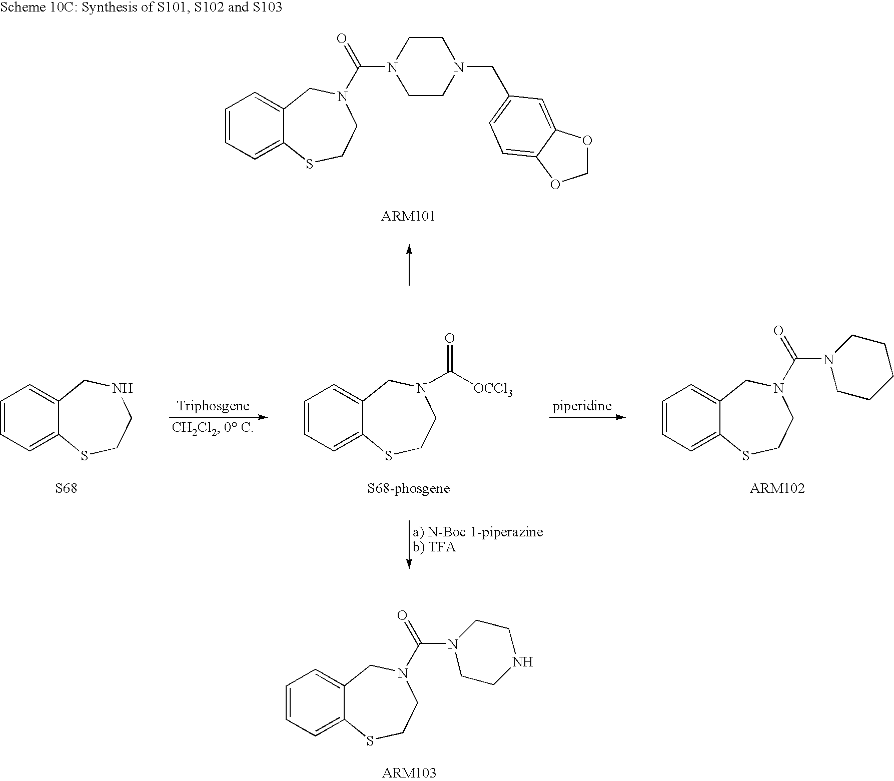

Synthesis of S55, S56, S58, S60-S63 (Scheme 11): The reaction mixture of S27 (25 mg, 0.1 mmol) and 4-(4-aminobenzyl)piperidine (19 mg, 0.1 mmol) in chloroform (5 ml) is stirred at room temperature for 2 days. After removal of solvent, the product S60 is purified by silica gel column chromatography as a white solid (36 mg, yield: 90%). S56, S58 and S60-63 can be synthesized by a similar method, using the appropriate amine.

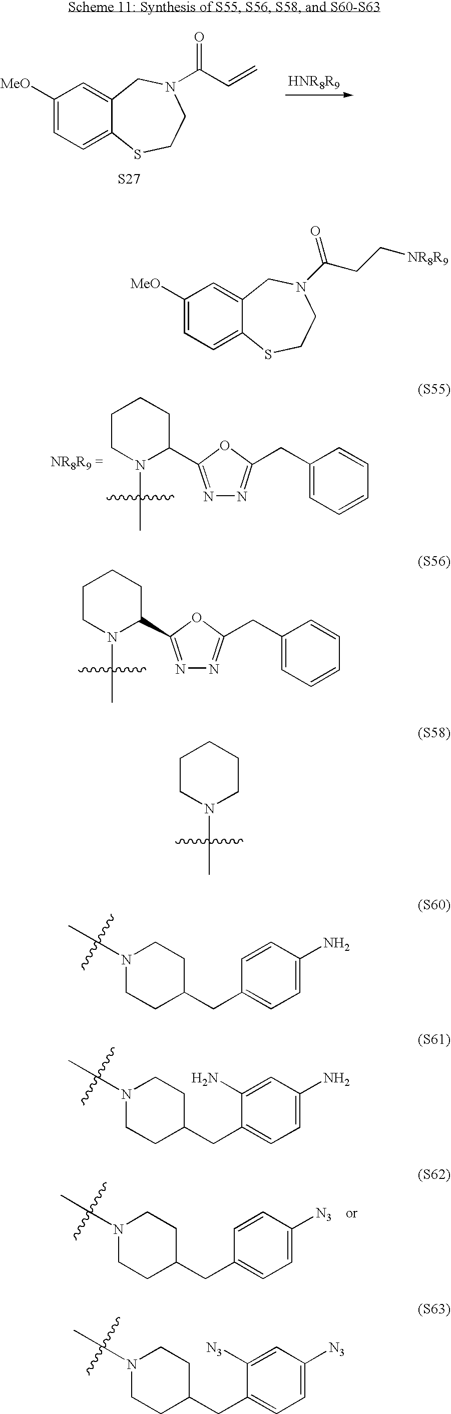

Synthesis of S85-S93 is accomplished as shown in Scheme 12. The following are examples of the synthesis.

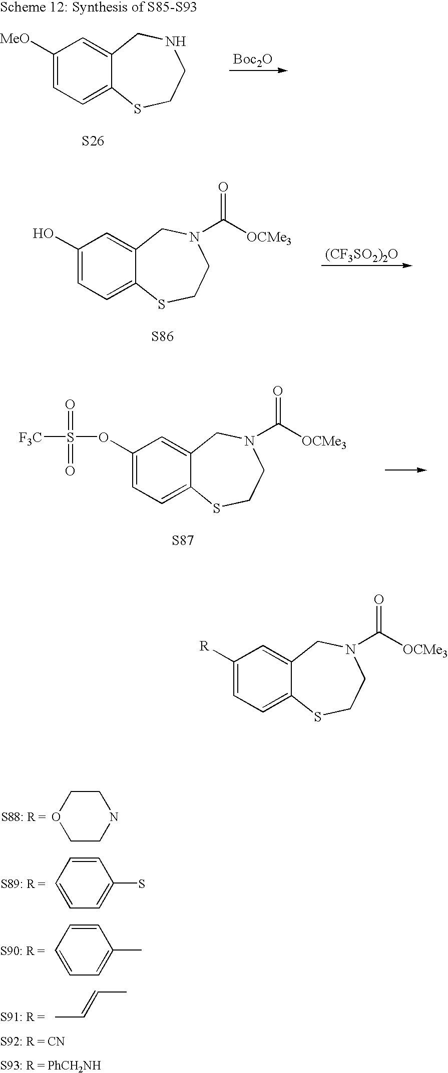

Synthesis of S85: A solution of S26 (10 mmol), di-tert-butyl dicarbonate (11 mmol), and triethylamine (12 mmol) in dichloromethane (100 ml) is stirred at room temperature for 5 hours. The reaction mixture is washed with saturated sodium bicarbonate solution (10 ml) and the aqueous layer is extracted with dichloromethane (2×15 ml). The combined organic layers are dried over magnesium sulfate and concentrated under vacuum to provide S85 as colorless oil (2.90 g, 98% yield).

Synthesis of S86: To a solution of S85 (2.36 g, 8 mmol) in dichloromethane (100 ml) at −78° C. is added BBr₃ (1.0 M solution in dichloromethane) (18 ml, 18 mmol) drop-wise. The solution is warmed to room temperature and the reaction mixture is quenched with methanol (100 ml) and concentrated under vacuum. The product S86 is purified by column chromatography.

Synthesis of S87: To a solution of S86 (6 mmol) in dichloromethane (40 ml) at 0° C. is added triethylamine (7 mmol) followed by trifluoromethylsulfonyl anhydride (7 mmol). The solution is stirred at room temperature for 30 minutes, and the reaction mixture is quenched with water (10 ml). The aqueous layer is extracted with dichloromethane (2×15 ml), and the combined organic layers are dried over magnesium sulfate and concentrated under vacuum. The crude product is purified by silica gel flash chromatography to provide S87 in 75% yield.

Synthesis of S88: A mixture of S87 (I mmol), morpholine (8 ml), tris(dibenzylideneacetone)dipalladium(0) (5 mol %), 2-(di-tert-butylphosphino)-biphenyl (20 mol %), and potassium phosphate (1.2 mmol) is heated at 80° C. in a sealed tube for 12 hours. The reaction mixture is cooled to room temperature, diluted with dichloromethane (50 ml), and washed with water (10 ml). The aqueous layer is extracted with dichloromethane (2×15 ml), and the combined organic layers are dried over magnesium sulfate and concentrated under vacuum. The crude product is purified by silica gel flash chromatography to give S88 in 81% yield.

Synthesis of S89: A solution of S87 (1 mmol), benzenethiol (2 mmol) and i-Pr₂NEt (2 mmol) in CH₃CN (20 ml) is heated at 80° C. for 18 hours. After cooling, ethyl acetate (30 ml) is added and then the reaction is washed with 1N HCl, water, and then 1N NaOH. After drying with Na₂SO₄, the solution is concentrated. The product S89 is purified by chromatography in 59% yield. Alternatively, S89 is synthesized by refluxing of S87 with benzenethiol in dioxane for 10 hours using i-Pr₂NEt/Pd₂(dba)₃/xantphos as catalyst.

Synthesis of S90: To a solution of S87 (1.0 mmol) in dioxane (10 mL) are added K₂CO₃ (2 mmol), phenylboronic acid (1 mmol), and Pd(Ph₃P)₄ (0.11 mmol), and the mixture is stirred at 90° C. for 16 hours. The reaction mixture is cooled to 25° C., diluted with CH₂Cl₂ (30 mL), washed with water (10 mL), and the organic phase is evaporated to dryness under vacuum. Purification by column chromatography gives S90 in 40% yield.

Synthesis of S91: S91 may be prepared under Heck conditions by treating S87 with propene in the presence of a metal catalyst e.g., Pd catalyst, a ligand and a base using a suitable solvent and temperature.

Synthesis of S92: To a solution of S87 (1.0 mmol) in DMF (5 mL) are added zinc cyanide (1 mmol) and Pd(Ph₃P)₄ (0.11 mmol). The reaction mixture is stirred and heated at 100° C. for 1 hour, followed by cooling, dilution with water (50 mL) and 2 M sulfuric acid (5 mL), and extraction with EtOAc (3×). The combined organic extracts are washed with brine (2×), dried over magnesium sulfate, filtered, and evaporated under vacuum. The product S92 is purified by silica gel column chromatography in 80% yield.

Synthesis of S93: S93 may be prepared from S87 by treating S87 with benzylamine in the presence of a catalyst, e.g., Pd, a suitable ligand (e.g., phosphine), and a suitable base (e.g., sodium tert-butoxide) in an appropriate solvent and temperature.

Synthesis of S94-S100 is accomplished as shown in Scheme 13. The following are examples of the synthesis.

Synthesis of S94: To a solution of S86 (1 mmol) in CH₂Cl₂ (10 ml) is added at 0° C. acetic anhydride (1.2 mmol) and triethylamine (1.3 mmol). The reaction mixture is stirred at room temperature overnight, then washed with H₂O. After drying with Na₂SO₄, the solvent is evaporated and the product S94 (98% yield by NMR) is used for the next reaction without further purification.

Synthesis of S95: To a stirred solution of S84 (0.5 mmol) in benzene (20 ml) is added anhydrous AlCl₃ (0.6 mmol) drop-wise. The reaction mixture is refluxed for 5 hours and poured on to crushed ice (10 g). After extraction and concentration, any deprotected intermediate is converted to S95 by reaction with Boc₂O, and the product S95 is purified by silica gel column chromatography in 83% yield.

Synthesis of S96: To a solution of S86 (0.1 mmol) in methanol (5 ml) is added NaI (10 mg, excess) and Chloramine-T (0.3 mmol). The reaction mixture is stirred for 30 minutes and quenched with Na₂S₂O₃ solution. The solvent is evaporated. The product is purified by silica gel column chromatography as a mixture of mono-iodinated or di-iodinated products in a combined yield of 60%.

Synthesis of S97: S86 (3 mmol) is added to concentrated H₂SO₄ (2 ml). To the stirred mixture is added, slowly, concentrated HNO₃ (2 ml) drop-wise. After 10 minutes, the reaction mixture is poured on to crushed ice (5 g) and neutralized with Na₂CO₃ to pH=7. The Boc-deprotected nitro intermediate is collected by extraction with EtOAc and converted to S97 by reaction with Boc₇O. Purification by silica gel column chromatography provides S97 in 78% yield.

Synthesis of S98: A mixture of S97 (2 mmol) and 10% Pd/C (0.1 g) in methanol (20 ml) is bubbled through with H₂ gas for 2 hours. After filtration and concentration, the amine product is used for the next reactions without further purification.

Synthesis of S99 and S100: S98 (1 mmol) is dissolved in aqueous HCl (2 mmol HCl, 10 ml H₂O). To this solution is added at 0° C. slowly a solution of sodium nitrite (1 mmol) in water (5 ml). The reaction mixture is stirred at 0° C. for 1 hour, then NaN₃ (2 mmol) in water (2 ml) is added drop-wise at 0° C. The resulting mixture is stirred at 0° C. for 1 hour and at room temperature overnight. The product is extracted with ethyl acetate and washed with saturated sodium bicarbonate and water, then treated with Boc₂O, and extracted and washed. The organic layer is dried over anhydrous sodium sulfate and concentrated to give crude product S98. Column purification on silica gel provide the product in 71% yield. Similarly, S99 is synthesized in 60% yield using NaBF₄ instead of NaN₃.

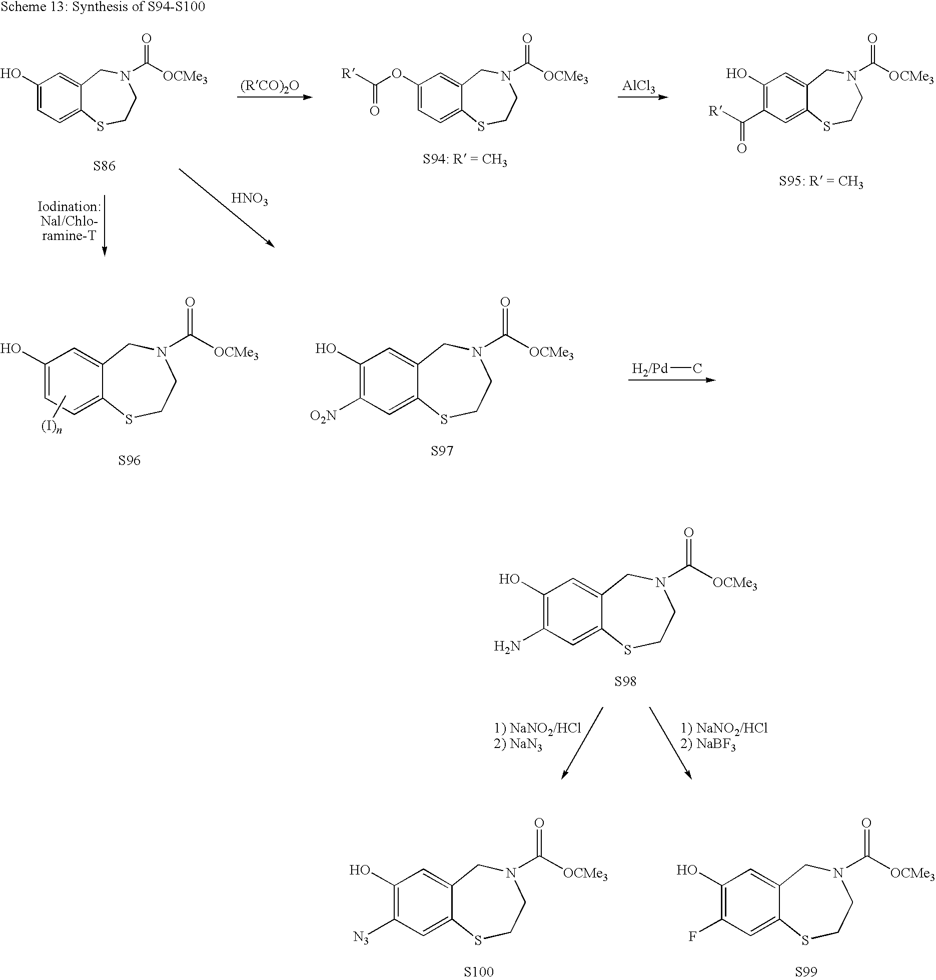

wherein n is 1-2.

Synthesis of S104 may be accomplished as shown in Scheme 14. The following is an example of the synthesis. A mixture of S36 (27 mg, 0.1 mmol), 50% H₂O₂ (1 ml), and MeOH (3 ml) is stirred at room temperature for 2 days to generate the S104 product. Mass spectroscopy (MS) is used to monitor the disappearance of S36 and the appearance of the product S104. The solvents are removed under reduced pressure, and the product is purified by re-crystallization. The final yield is 26 mg of S104 at 85% purity. The structure of the final product is determined by nuclear magnetic resonance (NMR) and/or MS.

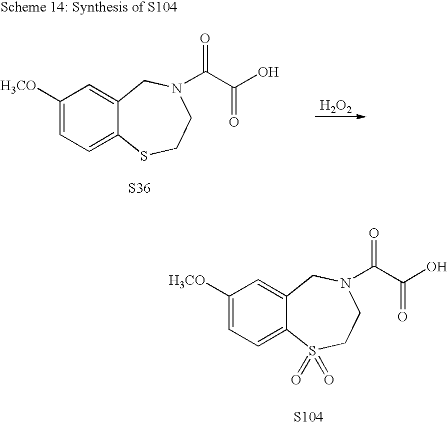

Synthesis of S107 may be accomplished as shown in Scheme 15. The following is an example of the synthesis: To S26 (180 mg, 0.92 mmol) in MeOH (20 ml) is added 30% CH₂O solution (1.5 ml, excess) and sodium cyanoborohydride (NaBCNH₃) (0.4 g, excess). The reaction mixture is stirred at room temperature, and the pH of the solution is maintained at around pH 4-5 by addition of a few drop of 1N HCl. After 3 hours, the solvents are removed under reduced pressure. The residue is dissolved in 20 ml ethyl acetate and washed with H₂O and saturated NaHCO₃ (2×10 ml). The solvents are removed and the S107 is purified by SiO₂ column chromatography to give a yield: 170 mg, 93%.

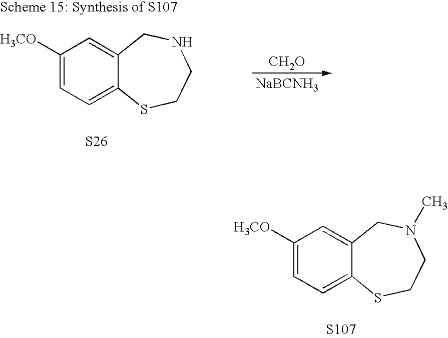

Synthesis of S108 may be accomplished as shown in Scheme 16. The following is an example of the synthesis: A mixture of N-benzyloxycarbonyl-glycine (Cbz-Gly, 129 mg, 0.61 mmol), Diisopropyl-carbodiimide (DIC, 90 mg, 0.71 mmol), N-hydroxysuccinimide (NHS, 70.4 mg, 0.71 mmol) in CH₂Cl₂ (50 ml) is stirred for 0.5 h at room temperature. To this mixture is added S26 (100 mg, 0.51 mmol) and the mixture is stirred at room temperature overnight. After washing with 1N HCl (2×10 ml) and saturated NaHCO₃ solution (2×10 ml), the solvents are removed by evaporation. The product S108 is purified by SiO₂ column chromatography, to give a yield of 120 mg, 61%.

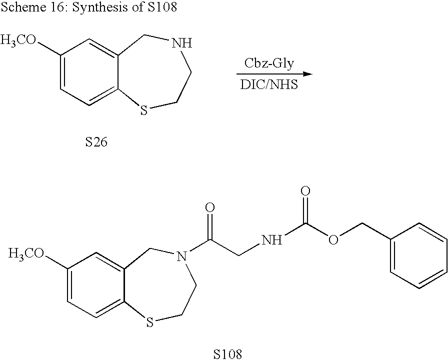

Synthesis of S109 may be accomplished as shown in Scheme 17. The following is an example of the synthesis: S108 (40 mg, 0.1 mmol) in CH₂Cl₂ (5 ml) is treated with 1 ml of 30% HBr/CH₃CO₂H. After stirring at room temperature overnight, the reaction mixture is evaporated under reduced pressure. The residue is dissolved in MeOH (3 ml) and treated with propylene oxide (1 ml). The solvents are removed under reduced pressure to provide crude S109 which is further purified by dissolving in 0.15 N HCl in H₂O solution (3.5 ml), followed by washing with ethyl acetate (3 ml) and evaporation. The yield of S109 is 28.3 mg, 95% (white powder. HCl salt).

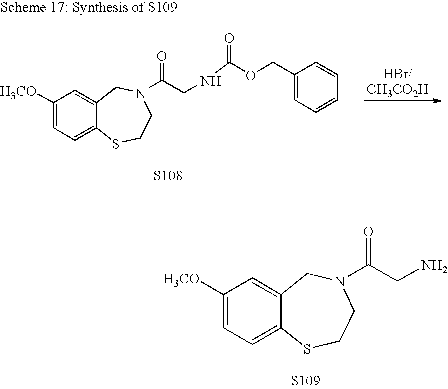

Synthesis of S110 may be accomplished as shown in Scheme 18. The following is an example of the synthesis: A mixture of S26 (100 mg, 0.51 mmol) methyl 1-bromoacetate (100 mg, 1.2 eq.) and pyridine (50 mg) in DMF (5 ml) is stirred at room temperature overnight. To this mixture, ethyl acetate (50 ml) is added and the reaction is washed with saturated NaHCO₃ solution (2×10 ml) and H₂O (2×10 ml). The product S110 as an oil is purified by SiO₂ column chromatography, to give a yield of 32 mg, 23%.

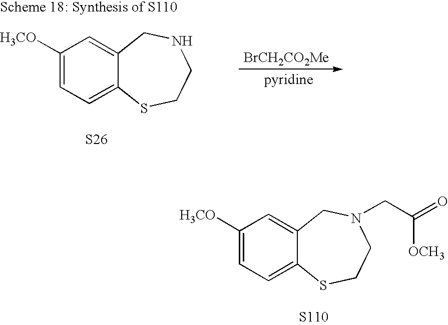

Synthesis of S111 may be accomplished as shown in Scheme 19. The following is an example of the synthesis: To S110 (16 mg, 0.06 mmol) in MeOH (2 ml) is added 1N NaOH (0.1 ml) and the mixture is stirred at room temperature overnight. The solvents are removed under reduced pressure and the residue is dissolved in H₂O (10 ml). The aqueous phase is washed with ethyl acetate (2×5 ml) and acidified with 1N HCl to pH=4. Removal of the solvents under reduced pressure provides crude S111. The NaCl is removed using ethanol to yield pure S111 as solid, having a yield of 13 mg, 87%.

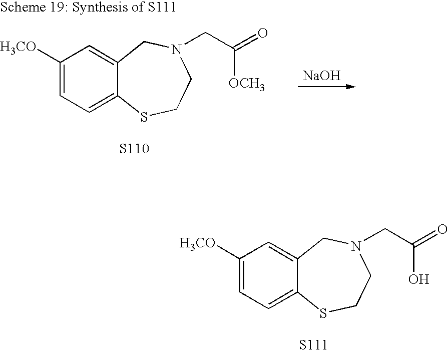

Synthesis of S112 may be accomplished as shown in Scheme 20. The following is an example of the synthesis: To a mixture of S26 (100 mg, 0.51 mmol) and pyridine (100 mg) in CH₂Cl₂ (20 ml), SO₂Cl₂ (89 mg, 1.2 eq.) is added drop-wise at 0° C. and the reaction is stirred at room temperature overnight. The solvents are removed under reduced pressure and the residue is dissolved in 5.5 ml NaOH solution (5 ml H₂O+0.5 ml 1N NaOH). The water solution is washed with ethyl acetate (2×5 ml), and acidified with 1N HCl to pH 4. The aqueous phase is extracted with ethyl acetate (3×5 ml) and the ethyl acetate phase is evaporated under reduced pressure to provide S112, as powder, with a yield of 9 mg.

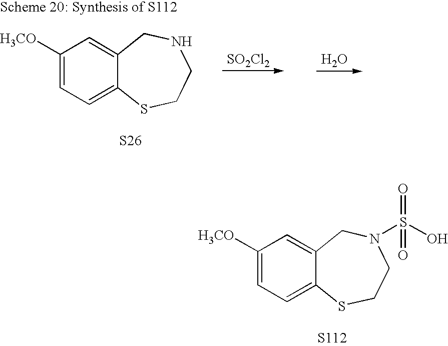

Synthesis of S113 may be accomplished as shown in Scheme 21. The following is an example of the synthesis: S107 (45 mg, 0.21 mmol) in ethyl acetate (2 ml) is treated with CH₃I (200 mg, excess). The mixture is stirred at room temperature overnight and the product S113, as white solid, is collected by filtration to give a yield of 69 mg, 97%.

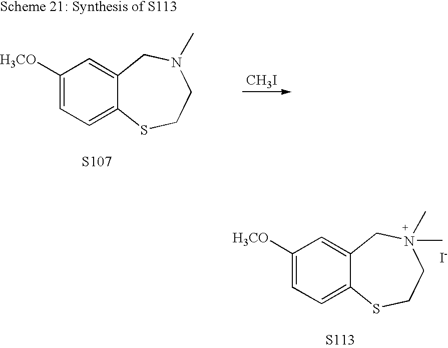

Synthesis of S114 may be accomplished as shown in Scheme 22. The following is an example of the synthesis: S26 (195 mg, 1 mmol) in CH₂Cl₂ (50 ml) is cooled to 0° C. To this solution, triphosgene (150 mg, 0.5 mmol) and pyridine (0.5 ml. excess) are added and stirred at 0° C. for 1 hour. Without purification, the resulting S26-phosgene in the reaction mixture is treated with N-Boc-1-piperazine (200 mg, 1.1 mmol) at 0° C. After stirring at 0° C. for 1 hour, the reaction mixture is washed with H₂O (2×10 ml), 1N HCl (2×10 ml), and saturated NaHCO₃ (2×10 ml), and the solvents are removed under reduced pressure. Purification by SiO₂ column chromatography provides S114 with a yield of 80%.

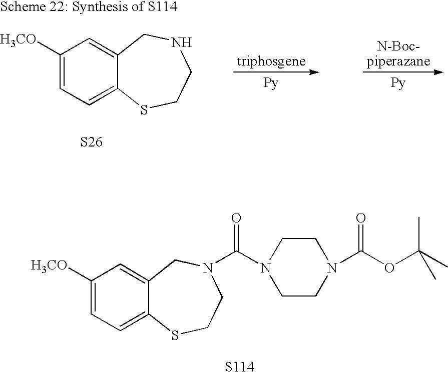

Synthesis of S115 may be accomplished as shown in Scheme 23. The following is an example of the synthesis: A mixture of S114 (200 mg, 0.49 mmol) and Lawesson's Reagent (400 mg) in toluene (50 ml) is stirred at 90° C. for 5 hours. The mixture is cooled to room temperature and washed with saturated NaHCO₃ (2×20 ml). The product S115 is purified by SiO₂ column chromatography to give a yield of 160 mg, 75%.

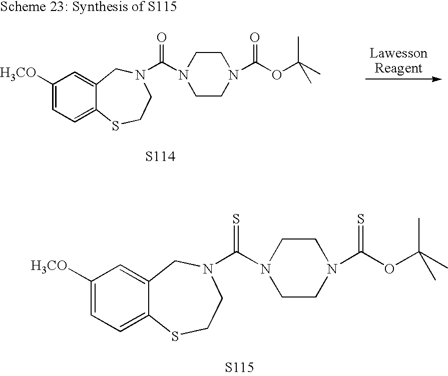

Synthesis of S116 may be accomplished as shown in Scheme 24. The following is an example of the synthesis: A mixture of S15 (10 mg, 0.02 mmol) and trifluoroacetic acid (TFA, 0.5 ml) in CH₂Cl₂ (10 ml) is stirred at room temperature for 2 hours. Evaporation of the solvents under reduced pressure produces S116 with yield of 6 mg, 92%.

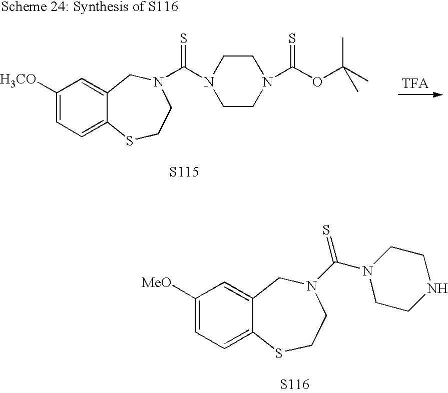

Synthesis of S117 may be accomplished as shown in Scheme 25. The following is an example of the synthesis: A solution of S57 (200 mg, 0.71 mmol) in CH₂Cl₂ (20 ml) is cooled to −78° C. To this, 1M BBr₃ in CH₂Cl₂ (1 ml) is added, and the mixture is stirred at −78° C. for 3 hours and then warmed to room temperature overnight. The mixture is washed with 1N HCl (2×10 ml) and H₂O (1×10 ml). After removal of the solvents, the product S117 is purified by SiO₂ column chromatography to give a yield of 60 mg, 33%.

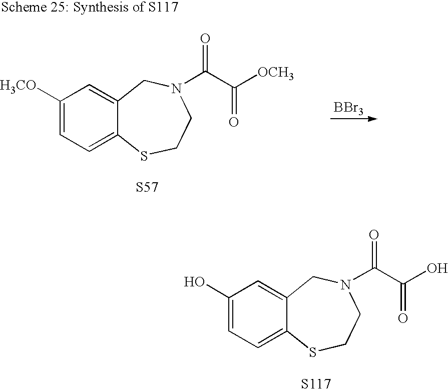

Synthesis of S118 may be accomplished as shown in Scheme 26. The following is an example of the synthesis: S26 (3.6 mg, 0.018 mmol) in CH₂Cl₂ (3 ml) was treated with BODIPY TMR-X, SE (Molecular Probes InC) (4 mg, 0.006 mmol) for 3 hours. The mixture is washed with 0.01 N HCl (2×1 ml) and saturated NaHCO₃ (2×1 ml). Removal of the solvents under reduced pressure yields pure S118 (98%).

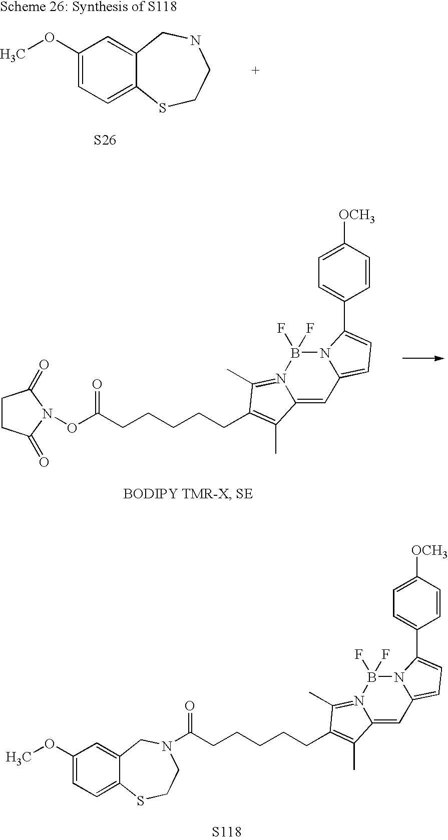

Synthesis of S119 may be accomplished as shown in Scheme 27. The following is an example of the synthesis: A mixture of S107 (50 mg, 0.24 mmol), 50% H₂O₂ (1 ml), and MeOH (3 ml) is stirred at room temperature for 2 days (mass spectrometry is used to monitor the disappearance of S107 and the formation of the product). The solvents are removed under reduced pressure to give S119, having a yield of 26 mg, 45%.

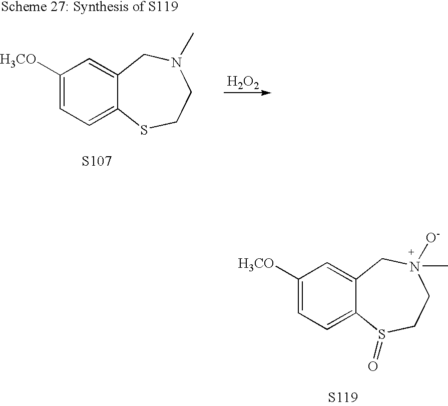

Synthesis of S120 and S121 may be accomplished as shown in Scheme 28. The following is an example of the synthesis: A mixture of S26 (195 mg, 1 mmol), benzyl bromide (1.1 mmol) and Na₂CO₃ (10 mmol) in DMF (10 ml) is stirred overnight. Ethyl acetate (30 ml) is added to the reaction, and then the reaction is washed with H₂O (4×10 ml). The organic phase is concentrated under reduced pressure and the residue is purified by column chromatography to give S121 as a white powder, with a yield of 280 mg, at 98%. S120 is similarly synthesized, but using 4-OH-benzyl bromide instead of benzyl bromide.

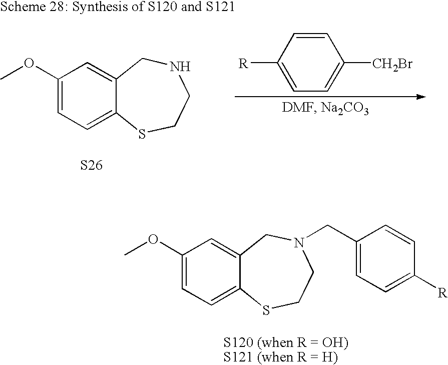

Synthesis of S122 (LB21300-30). The following is an example of the synthesis: To a cold solution of compound S26 (250 mg, 1.28 mmol, 1 equivalent) in CH₂Cl₂ (50 mL) at 0° C. is added DIEA (0.67 mL, 3.8 mmol, 3.0 equivalent), followed by acetoxyacetyl chloride (0.17 mL, 1.58 mmol, 1.24 equivalent). Then, the reaction mixture is stirred at 0° C. for 20 min, diluted with 1.0 M HCl aqueous solution (100 mL) and extracted by CH₂Cl₂ (3×50 mL). The combined organic layers are washed (H₂O, brine), dried (Na₂SO₄), filtered, and evaporated to dry. The crude product is purified by chromatography on a silica gel column, eluting with a gradient increasing in polarity from 0 to 50% petroleum in ethyl acetate. Relevant fractions are combined to give the product (350 mg, 93%).

Synthesis of S123 (LB21300-34). The following is an example of the synthesis: To a solution of compound S122 (287 mg, 0.97 mmol, 1 equivalent) in MeOH (5 mL) and THF (8 mL) at 23° C. is added LiOH (140 mg, 3.33 mmol, 3.44 equivalent in H₂O 5 mL). The reaction mixture is stirred at 23° C. for 20 minutes, diluted with 1.0 M HCl aqueous solution (100 mL) and extracted by CH₂Cl₂ (3×50 mL). The combined organic layers were washed (H₂O, brine), dried (Na₂SO₄), filtered and evaporated to dryness. The crude product is purified by chromatography on a silica gel column, eluting with a gradient increasing in polarity from 0 to 70% petroleum in ethyl acetate. Relevant fractions are combined to give S123 (244 mg, 100%).

Synthesis of Radio-Labeled JTV-519 (Scheme 29): To prepare radio-labeled JTV-519, JTV-519 is demethylated at the phenyl ring using BBr₃ to give phenol compound (21). The phenol compound (21) is re-methylated with a radio-labeled methylating agent (3H-dimethyl sulfate) in the presence of a base (for example NaH) to provide ³H-labeled JTV-519.

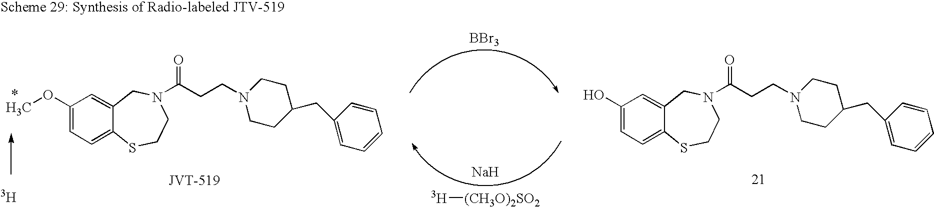

While the foregoing invention has been described in some detail for purposes of clarity and understanding, it will be appreciated by one skilled in the art, from a reading of the disclosure, that various changes in form and detail may be made without departing from the true scope of the invention in the appended claims.

What is claimed is:

1. A method of synthesis which comprises:
(a) treating a compound having the formula: 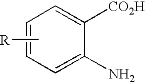
wherein R is H, OR₁, SR₁, N(R₁)₂, alkyl, or halide, and R₁ is independently at each occurrence alkyl, aryl, or H, with a diazotizing agent and a disulfide under conditions sufficient to form a compound having formula: 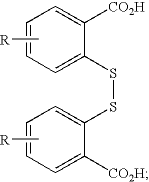
(b) treating the compound formed in (a) with a chloride and a chloroethylamine, under conditions sufficient to form a compound having formula: 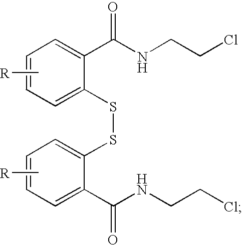
(c) treating the compound formed in (b) with a reducing agent and a base under conditions sufficient to form a compound having formula: 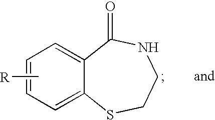
(d) treating the compound formed in (c) with a reducing agent under conditions sufficient to form a compound having formula:

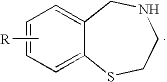

2. The method of claim 1, wherein the compound in (a) having the formula: 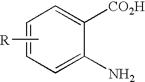
is synthesized by treating a compound having the formula: 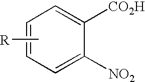
with a reducing agent, optionally in the presence of a catalyst, under conditions sufficient to form the compound in (a).

3. The method of claim 1, wherein
R is at position 7 of the benzothiazepine ring, and the compound formed in (d) has the structure: 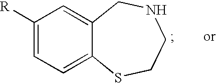
the diazotizing agent in reaction (a) is NaNO₂; or
the disulfide in reaction (a) is Na₂S₂; or the chloride in reaction (b) is SOCl₂; or
the base in (c) is triethyl amine a carbonate, sodium hydride, diisopropylamine (DIPEA) or N-methylmorpholine (NMM); or
the reducing agent in (c) is trimethylphosphine or sodium dithionite; or
the reducing agent in treatment (d) is LiAlH₄.

4. The method of claim 1 which further comprises:
(e1) reacting the compound formed in (d) 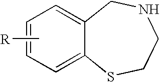
with a compound of formula R₂SO₂Cl under conditions sufficient to form a compound of the formula: 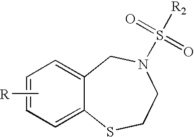
wherein R₁ is CH₂═CH—, Me, p-Me-C₆H₄, or —NH-2-Pyridyl; or
(f1) reacting the compound formed in (d) 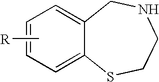
with a compound of formula R₂SO₂Cl under conditions sufficient to form a compound of the formula: 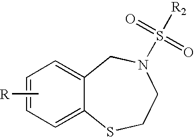
wherein R₂ is CH₂═CH—; and reacting the compound thus formed with a compound of formula HNR₃R₄, wherein NR₃R₄ is 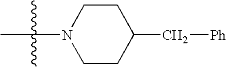
or NBu₂, under conditions sufficient to form a compound of the formula: 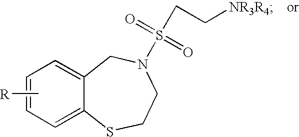
(g1) reacting the compound formed in (d) 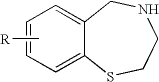
with SO₂Cl₂ and a base under conditions sufficient to form a compound of the formula: 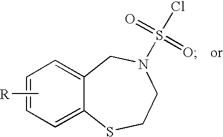
(h1) reacting the compound formed in (d) 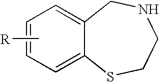
with SO₂Cl₂ and a base under conditions sufficient to form a compound of the formula: 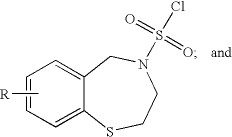
hydrolyzing that compound under conditions sufficient to obtain a compound of the formula:

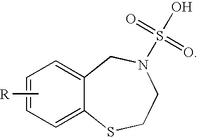

5. The method of claim 1 which further comprises:
(e2) reacting the compound formed in (d) 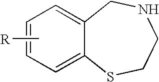
with a compound of formula R₅COX₁, wherein R₅ is —CH₁₂I, Ph-, CH₂═CH—, 4-N₃-2-OH—C₆H₅ or 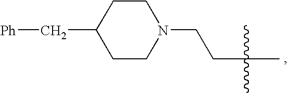
and X₁ is Cl or NHS, under conditions sufficient to form a compound of the formula: 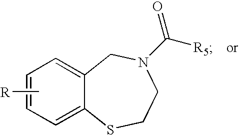
(f2) forming the compound 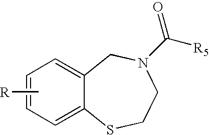
by the method of (e2) and treating that compound with hydrogen peroxide or m-CPBA under conditions sufficient to form a compound of the formula: 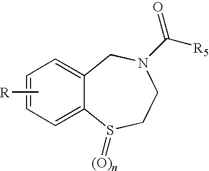
wherein n is 1 or 2; or
(g2) forming the compound 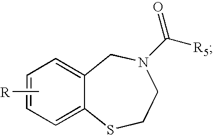
by the method of (e2), wherein R₅ is CH₂═CH—, and reacting that compound with a compound of formula HNR₈R₉, wherein NR₈R₉ is 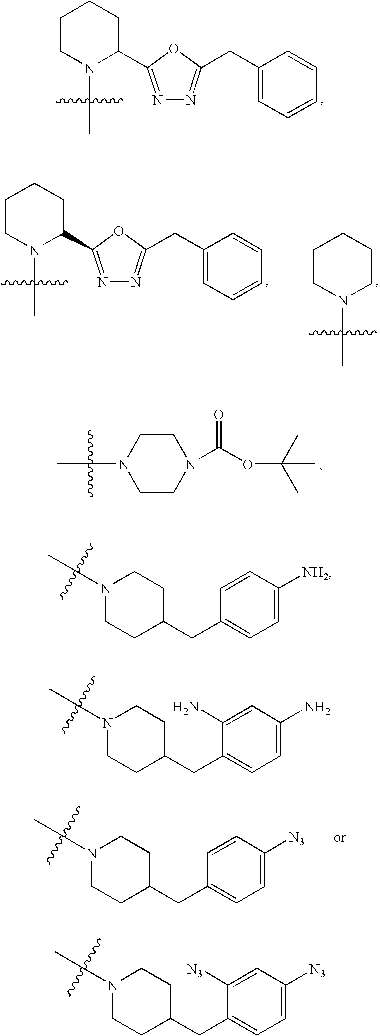
conditions sufficient to form a compound of the formula:

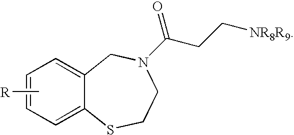

6. The method of claim 1 which further comprises:
(e3) reacting the compound formed in (d) 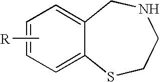
with an acid chloride of formula Cl—C(═O)ORₐₐ under conditions sufficient to form a compound of the formula: 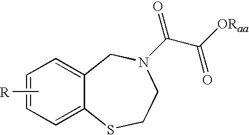
wherein Rₐₐ is C₁-C₄ alkyl or aryl; or
(f3) reacting the compound formed in (d) 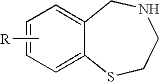
with an acid-chloride of formula Cl—C(═O)ORₐₐ under conditions sufficient to form a compound of the formula: 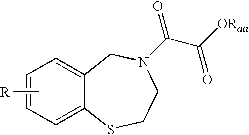
wherein Rₐₐ is C₁-C₄ alkyl or aryl; and reacting that compound with an acid or a base under conditions sufficient to form a compound of the formula: 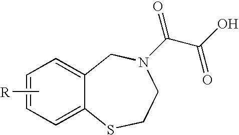
or its salts; or
(g3) reacting the compound formed in (d) 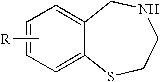
with an acid chloride of formula Cl—C(═O)ORₐₐ under conditions sufficient to form a compound of the formula: 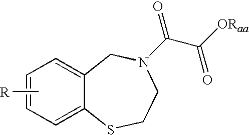
wherein Rₐₐ is C₁-C₄ alkyl or aryl; and reacting that compound with an acid or a base under conditions sufficient to form a compound of the formula: 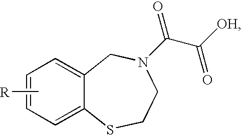
or a salt, and in the case a salt is formed, contacting the salt with an acid under conditions sufficient to form the compound of formula 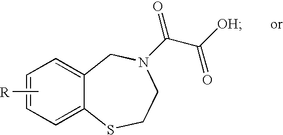
(h3) forming the compound 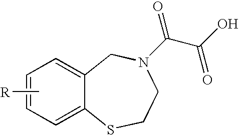
by the method of (f3) or (g3) and reacting that compound with hydrogen peroxide under conditions sufficient to form a compound of the formula: 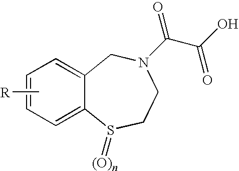
or its salts, wherein n is 1 or 2; or
(i3) forming the compound 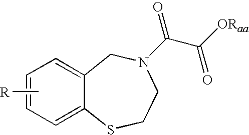
by the method of (f3) or (g3), wherein R is methoxy and Rₐₐ is methyl, with BBr₃ under conditions sufficient to form a compound of formula: 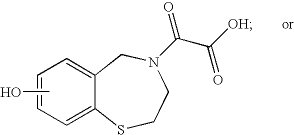
(j3) forming the compound 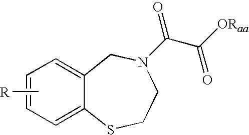
by the method of (f3) or (g3), and treating that compound with thionyl chloride or oxalyl chloride under conditions sufficient to form a compound of the formula: 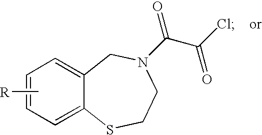
(k3) forming the compound 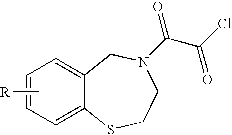
by one of the methods of 03), and reacting that compound with:
(1) cystamine and a base under conditions sufficient to form a compound of the formula: 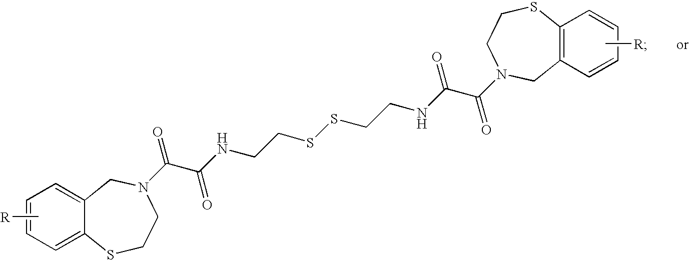
(2) a compound of the formula HX₂, wherein X₂ is OCH₃, NHEt, NHPh, NH₂, or NHCH₂-4-pyridine, under conditions sufficient to form a compound of the formula: 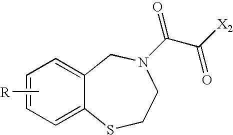
wherein X₂ is defined herein; or
(3) cystamine and a base to form S36-cystamine; and reacting the S36-cystamine with an NHS activated ester of an azido compound under conditions sufficient to form a compound of the formula: 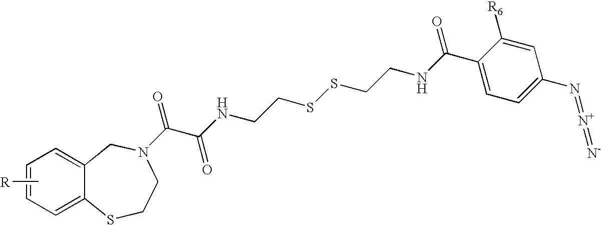
wherein R₆ is NO₂ or OH.

7. The method of claim 6, wherein
Rₐₐ is methyl or ethyl; or
the acid in reaction (f3) or (g3) is trifluoroacetic acid (TFA) or hydrochloric acid (HCl); or
the base in reaction (f3) is NaOH, KOH and LiOH; or
R is methoxy; or
R is at position 7 of the benzothiazepine ring and the compound has the structure; 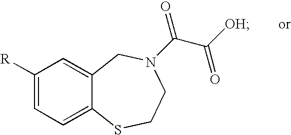
the compound of formula 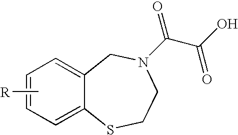
is further purified by dissolving the compound in water to form an aqueous solution; washing the aqueous solution with an organic solvent; acidifying the aqueous solution; extracting the compound from the aqueous solution using an organic solvent to form an extract; and removing the organic solvent from the extract.

8. The method of claim 1 which further comprises
(e4) reacting the compound formed in (d) 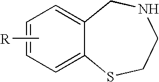
with:
(1) 4-nitrophenyl chloroformate under conditions sufficient to form a compound of the formula: 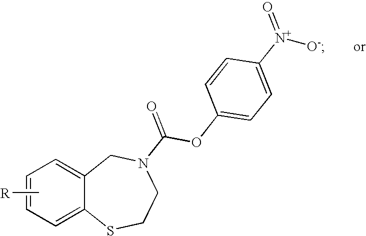
(2) triphosgene and a base to form a compound of the formula 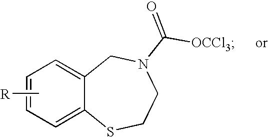
(f4) reacting the compound formed in (d) with:
(1) 4-nitrophenyl chloroformate under conditions sufficient to form a compound of the formula: 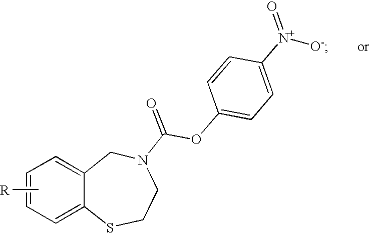
(2) triphosgene and a base to form a compound of the formula 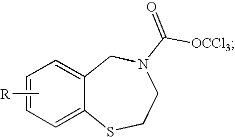
reacting either compound with an amine of formula HNR₇ₐR_{7b}, wherein NR₇ₐR_{7b} is —NH₂, —NEt₂, —NHCH₂Ph, NHOH, —NH₂, —NEt₂, —NHCH₂Ph, —NHOH, 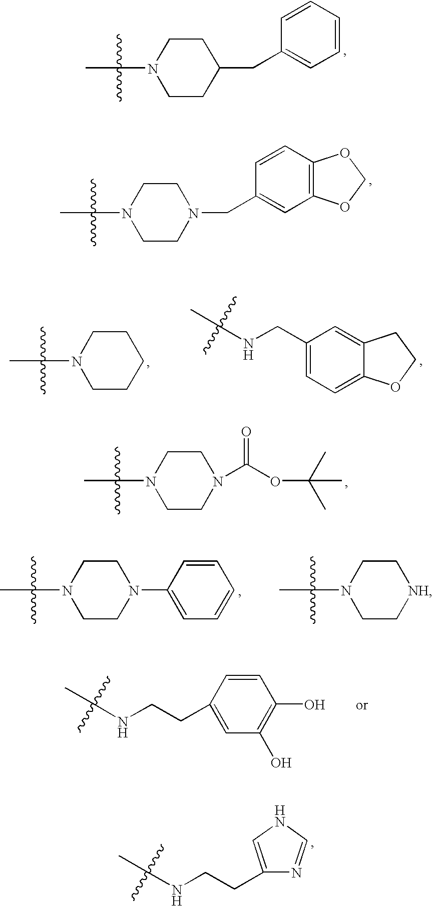
under conditions sufficient to form a compound of the formula: 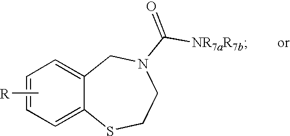
(g4) reacting an amine of formula HNR₇ₐR_{7b} wherein NR₇ₐR_{7b} is as defined herein with triphosgene under conditions sufficient to form a compound of formula Cl₃CO(C═O)NR₇ₐR_{7b}, and reacting that compound with the compound formed in (d) 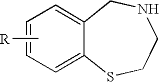
under conditions sufficient to form a compound of the formula: 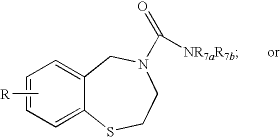
(h4) preparing the compound 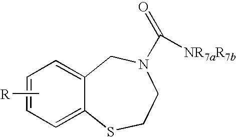
by the methods of (f4) or (g4), wherein NR₇ₐR_{7b} is 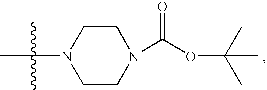
and reacting that compound with a Lawesson's Reagent under conditions sufficient to form a compound of the formula 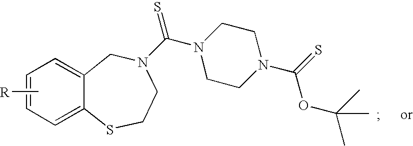
(i4) preparing the compound 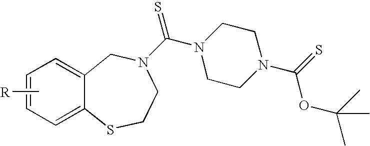
by the method of (h4) and reacting that compound with an acid under conditions sufficient to form a compound of the formula:

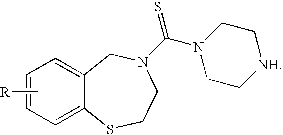

9. The method of claim 1 which further comprises:
(e5) reacting the compound formed in (d) 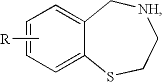
wherein R is at position 7 of the benzothiazepine ring, with Boc₂O or Boc-Cl under conditions sufficient to form a compound of the formula: 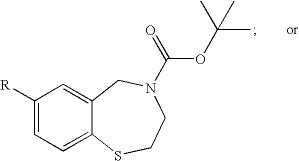
(f5) forming the compound 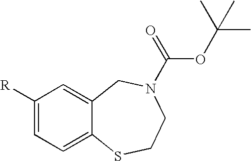
by the method of (e5), wherein R⁻ is methoxy, and reacting that compound with BBr₃ under conditions sufficient to form a compound of the formula: 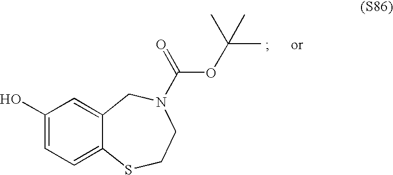
(g5) forming the compound S86 by the method of (f5) and reacting that compound with trifluoromethylsulfonyl anhydride under conditions sufficient to form the compound S87 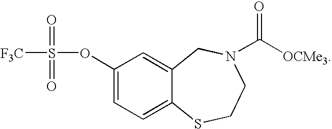
(h5) forming the compound S87 by the method of (g5) and reacting that compound with tris(dibenzylideneacetone)dipalladium(0), 2-(di-tert-butylphosphino)-biphenyl, and K₂PO₃, under conditions sufficient to form the compound S88 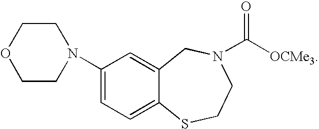
(i5) forming the compound S87 by the method of (g5) and reacting that compound with benzenethiol and a catalyst, under conditions sufficient to form compound S89 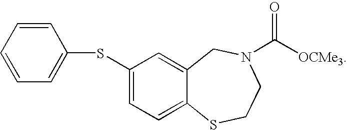
(j5) forming the compound S87 by the method of (g5) and reacting that compound with a base, phenylboronic acid, and a catalyst, under conditions sufficient to form compound S90 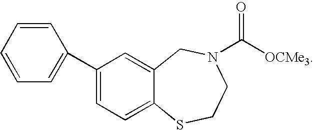
(k5) forming the compound S87 by the method of (g5) and reacting that compound with propene in the presence of a metal catalyst, a ligand and a base, under conditions suitable to form the compound S91 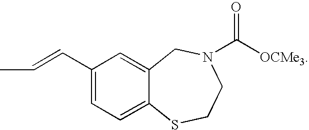
(l5) forming the compound S87 by the method of (g5) and reacting that compound with zinc cyanide and a catalyst, under conditions sufficient to form compound S92 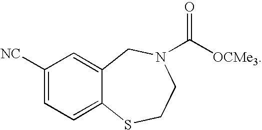
(m5) forming the compound S87 by the method of (g5) and reacting that compound with benzylamine in the presence of a catalyst, a ligand and a base, under conditions suitable to form the compound S93 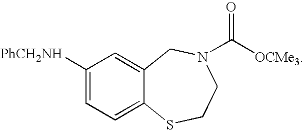
(n5) forming the compound S86 by the method of (f5) and reacting that compound with acetic anhydride under conditions sufficient to form compound S94 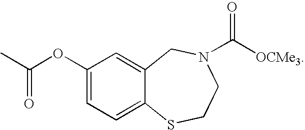
(o5) forming the compound S94 by the method of (n5) and reacting that compound with AlCl₃ under conditions sufficient to form compound S95 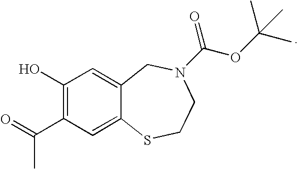
(p5) forming the compound S86 by the method of (f5) and reacting that compound with NaI and Chloramine-T under conditions sufficient to form compound S96 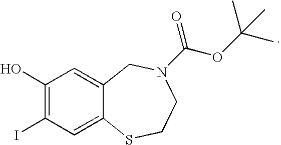
(q5) forming the compound S86 by the method of (f5) and reacting that compound with H₂SO₄ to form a sulfuric acid mixture, and then adding HNO₃ to the sulfuric acid mixture thus formed under conditions sufficient to form the compound S97 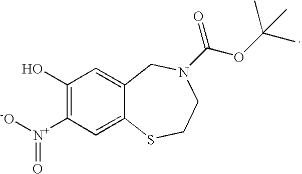
(f5) forming the compound S97 by the method of (q5) and hydrogenating that compound under conditions sufficient to form compound S98 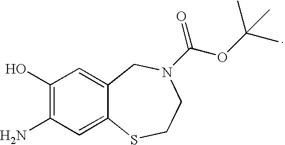
(s5) forming the compound S98 by the method of (f5) and reacting that compound with sodium nitrite and NaBaF₄ under conditions sufficient to form compound S99 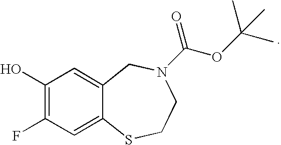
(t5) forming the compound S98 by the method of (f5), dissolving that compound in an acidic solution to form a mixture, and then reacting the mixture with sodium nitrite and NaN₃ under conditions sufficient to form compound S100

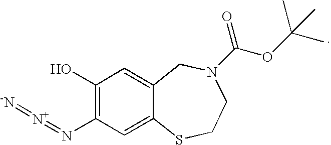

10. The method of claim 1 which further comprises:
(e6) reacting the compound formed in (d) 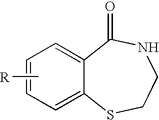
with a compound of formula to produce a compound of formula: 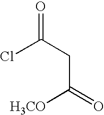 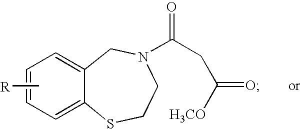
(f6) forming the compound 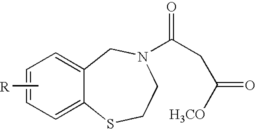
by the method of (e6) and reacting that compound with an acid or a base under conditions sufficient to form a compound of the formula:

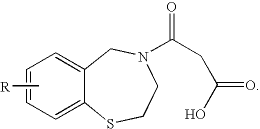

11. The method of claim 1 which further comprises:
(e7) reacting the compound formed in (d) 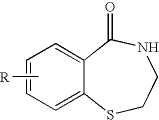
with formaldehyde (CH₂O) and a reducing agent under conditions sufficient to form a compound of the formula: 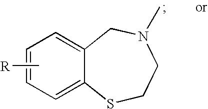
(f7) forming the compound 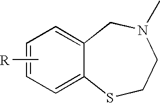
by the method of (e7) and reacting that compound with CH₃X₃ under conditions sufficient to form a compound of the formula: 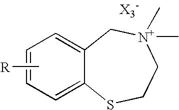
wherein X₃ is a halogen selected from F, Cl, Br and I; or
(g7) forming the compound 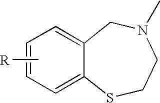
by the method of (e7) and reacting that compound with hydrogen peroxide under conditions sufficient to form a compound of the formula:

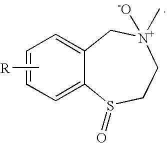

12. The method of claim 11, wherein R is methoxy and is located at position 7 of the benzothiazepine ring, or wherein the reducing agent in (a) is sodium cyanoborohydride (NaBCNH₃) or sodium triacetoxyborohydride.

13. The method of claim 1 which further comprises:
(e8) reacting the compound formed in (d) 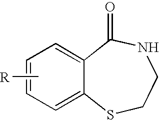
with N-benzyloxycarbonyl-glycine (Cbz-Gly) under conditions sufficient to form a compound of the formula: 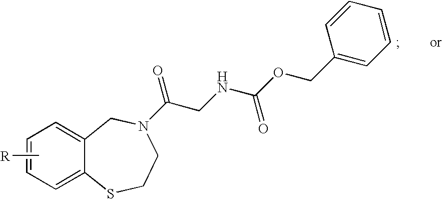
(f8) forming the compound 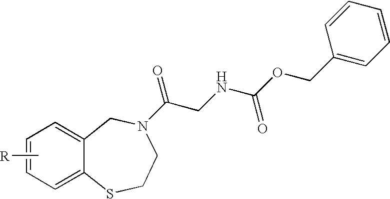
by the method of (e8) and treating that compound with an acid under conditions sufficient to form a compound of the formula:

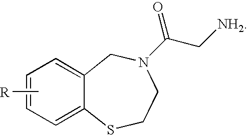

14. The method of claim 1 which further comprises:
(e9) reacting the compound formed in (d) 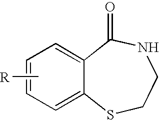
with a compound of formula: 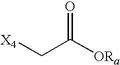
wherein X₄ is a halogen or a sulfonate and Rₐ is a C₁-C₄ alkyl under conditions sufficient to form a compound of the formula: 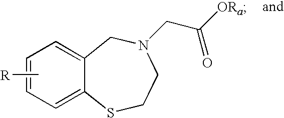
(f9) forming the compound 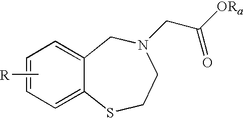
by the method of (e9) and treating that compound with an acid or a base under conditions sufficient to form a compound of the formula: 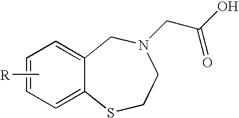
or a salt thereof.

15. The method of claim 1 which further comprises
(e10) treating the compound formed in (d) 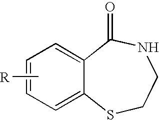
with BODIPY TMR-X under conditions sufficient to form a compound of the formula: 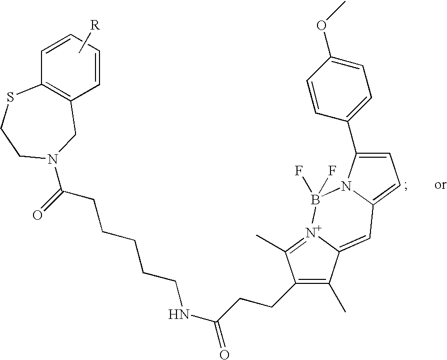
(f10) reacting the compound formed in (d) 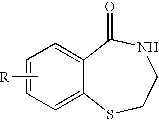
with 4-OH-benzyl bromide or benzyl bromide under conditions sufficient to form a compound of the formula: 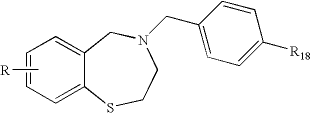
wherein R₁₈ is OH or H; or
(g10) reacting the compound formed in (d) 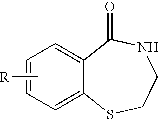
first with DIEA and then with acetoxyacetyl chloride under conditions sufficient to form a compound of the formula: 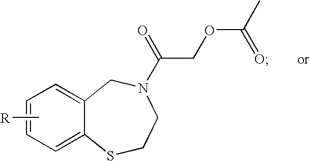
(h10) forming the compound 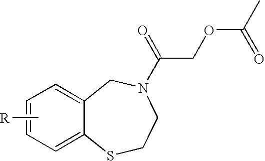
by the method of (g10) and reacting that compound with a base under conditions sufficient to form a compound of the formula: 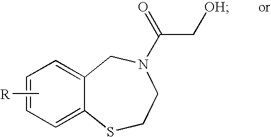
(i10) reacting the compound formed in (d) 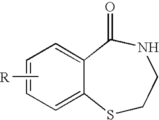
with a compound of formula C₆H₄—NCX₅, wherein X₅ is O or S, under conditions sufficient to form a compound of the formula: 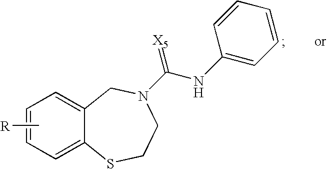
(j10) reacting the compound formed in (d) 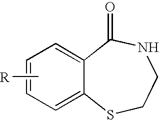
with phenyl methoxyphosphonyl chloride (Ph(MeO)P(O)Cl) in a solvent to form a reaction mixture, removing the solvent from the reaction mixture to form a residue, and purifying and separating the product into the isomers of compounds of the formulae: 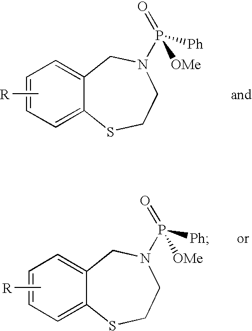
(k10) reacting the compound formed in (d) 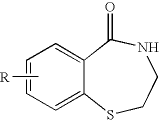
with a compound of formula ClOC—X₆—COCl, wherein X₆ is —CH₂—CH₂— or 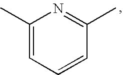
under conditions sufficient to form a compound of the formula:

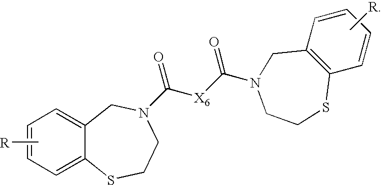

16. A method of synthesis of organic compounds from the starting compound 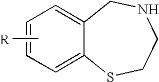
wherein R is H, OR₁, SR₁, N(R₁)₂, alkyl, or halide, and R₁ is independently at each occurrence alkyl, aryl, or H, which comprises:
(A-1) reacting the starting compound 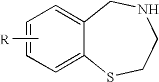
with a compound of formula R₅COX₁, wherein R₅ is CH₂I, Ph-, CH₂═CH—, 4-N₃-2-OH—C₆H₅ or 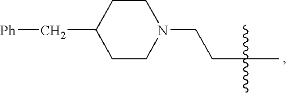
and X₁ is Cl or NHS, under conditions sufficient to form a compound of the formula: 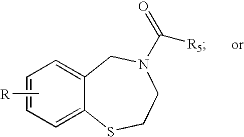
(B-2) forming the compound 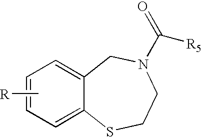
by the method of (A-1) and treating that compound with hydrogen peroxide or m-CPBA, under conditions sufficient to form a compound of the formula: 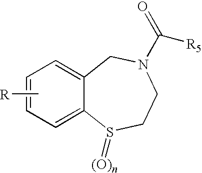
wherein n is 1 or 2, or
(C-1) forming the compound 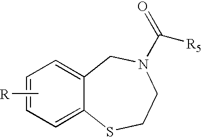
by the method of (A-1), wherein R₅ is CH₂═CH—, and reacting that compound with a compound of formula HNR₈R₉, wherein NR₈R₉ is 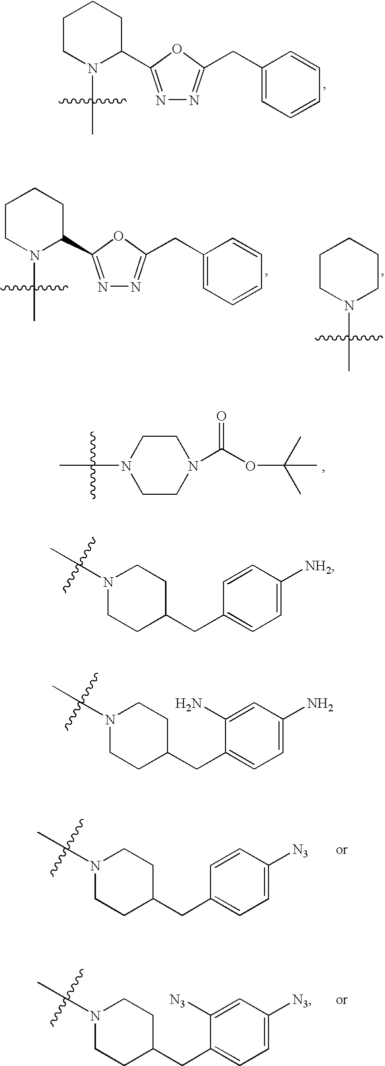
under conditions sufficient to form a compound of the formula: 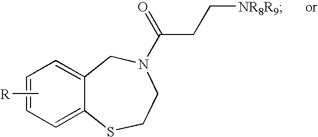
(D-1) reacting the starting compound 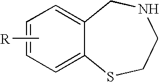
with an acid chloride of formula Cl—C(═O)ORₐₐ, under conditions sufficient to form a compound of the formula: 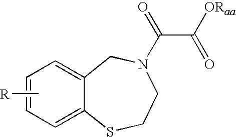
wherein Rₐₐ is C₁-C₄ alkyl or aryl; or
(E-1) forming the compound 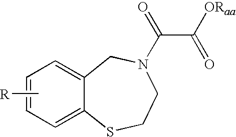
by the method of (D-1) and reacting that compound with an acid or base under conditions sufficient to form a compound of the formula: 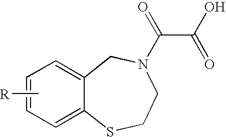
or its salts; or
(F-1) forming the compound 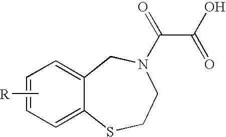
by the method of (E-1) in salt form, and adding an acid to the salt to form the compound of formula 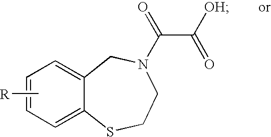
(G-1) forming the compound 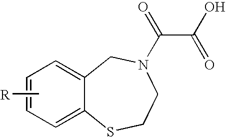
by the method of (E-1) or (F-1), and reacting that compound with hydrogen peroxide under conditions sufficient to form a compound of the formula: 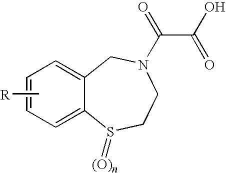
or its salts, wherein n is 1 or 2; or
(H-1) forming the compound 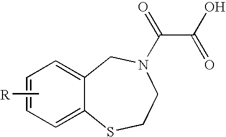
by the method of (E-1) or (F-1), wherein R is methoxy and Rₐₐ is methyl with BBr₃, and reacting that compound under conditions sufficient to form a compound of formula: 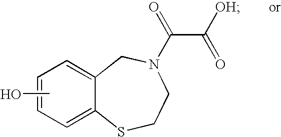
(I-1) forming the compound 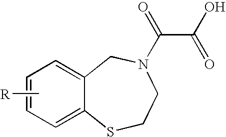
by the method of (E-1) or (F-1) and reacting that compound with thionyl chloride or oxalyl chloride under conditions sufficient to form a compound of the formula: 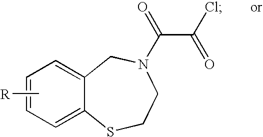
(J-1) forming the compound 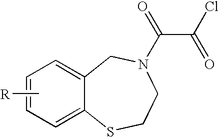
by the method of (I-1) and reacting that compound with:
(1) cystamine and a base under conditions sufficient to form a compound of the formula: 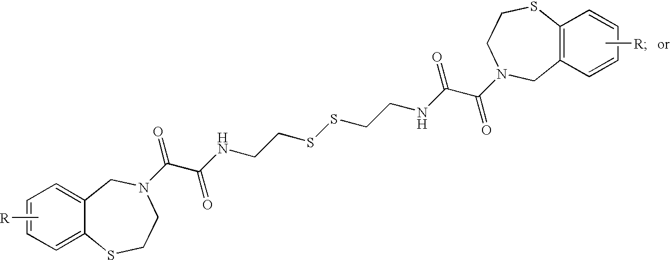
(2) a compound of the formula HX₂, wherein X₂ is OCH₃, NHEt, NHPh, NH₂, or NHCH₂-4-pyridine, under conditions sufficient to form a compound of the formula: 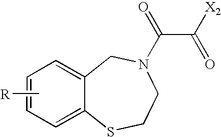
wherein X₂ is as defined herein; or
(3) cystamine and a base to form S36-cystamine; and reacting the S36-cystamine with an NHS activated ester of an azido compound under conditions sufficient to form a compound of the formula: 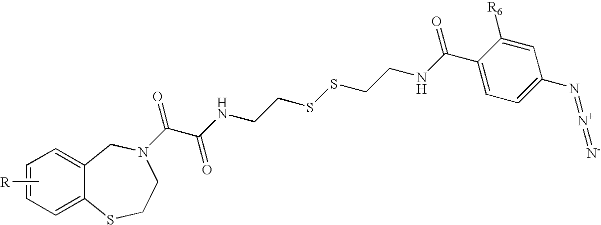
wherein R₆ is NO₂ or HO.

17. The method of claim 16, wherein:
Rₐₐ is methyl or ethyl; or
the acid in reaction (B-1) or (C-1) is trifluoroacetic acid (TFA) or hydrochloric acid (HCl); or
the base in reaction (B-1) is NaOH, KOH and LiOH: or R is methoxy; or
R is at position 7 of the benzothiazepine ring and the compound has the structure; 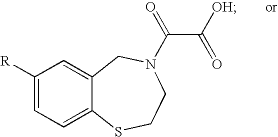
the compound of formula 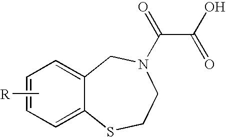
is further purified by dissolving the compound in water to form an aqueous solution; washing the aqueous solution with an organic solvent; acidifying the aqueous solution; extracting the compound from the aqueous solution using an organic solvent to form an extract; and removing the organic solvent from the extract.

18. A method of synthesis of organic compounds from the starting compound 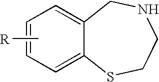
wherein R is H, OR₁, SR₁, N(R₁)₂, alkyl or halide, and R₁ is independently at each occurrence alkyl, aryl, or H, which comprises:
(A-2) reacting the starting compound 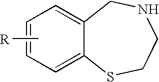
with a compound of formula R₂SO₂Cl under conditions sufficient to form a compound of the formula: 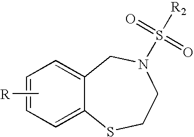
wherein R₂ is CH₂═CH—, Me, p-Me-C₆H₄, or —NH-2-Pyridyl; or
(B-2) forming the compound 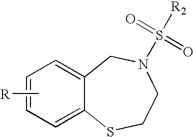
by the method of (A-2), wherein R₂ is CH₂═CH—, and reacting that compound with a compound of formula HNR₃R₄, wherein NR₃R₄ is 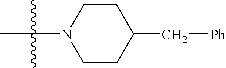
or NBu₂, under conditions sufficient to form a compound of the formula: 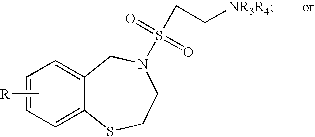
(C-2) reacting the starting compound 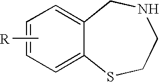
with SO₂Cl₂ and a base, under conditions sufficient to form a compound of the formula: 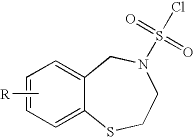
and hydrolyzing that compound to obtain a compound of the formula: 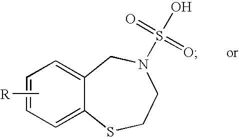
(D-2) reacting the starting compound 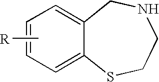
with
(1) 4-nitrophenyl chloroformate under conditions sufficient to form a compound of the formula: 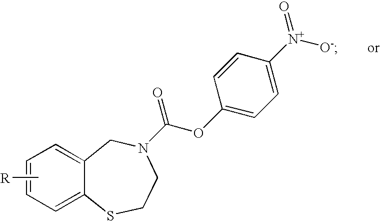
(2) triphosgene and a base to form a compound of the formula 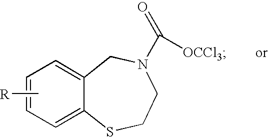
(E-2) reacting the starting compound 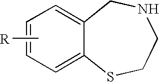
with
(1) 4-nitrophenyl chloroformate under conditions sufficient to form a compound of the formula: 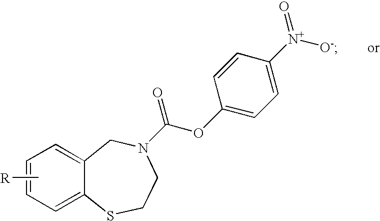
(2) triphosgene and a base to form a compound of the formula 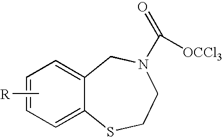
and reacting either compound with an amine of formula HNR₇ₐR_{7b}, wherein NR₇ₐR_{7b} is —NH₂, —NEt₂, —NHCH₂Ph, —NHOH, 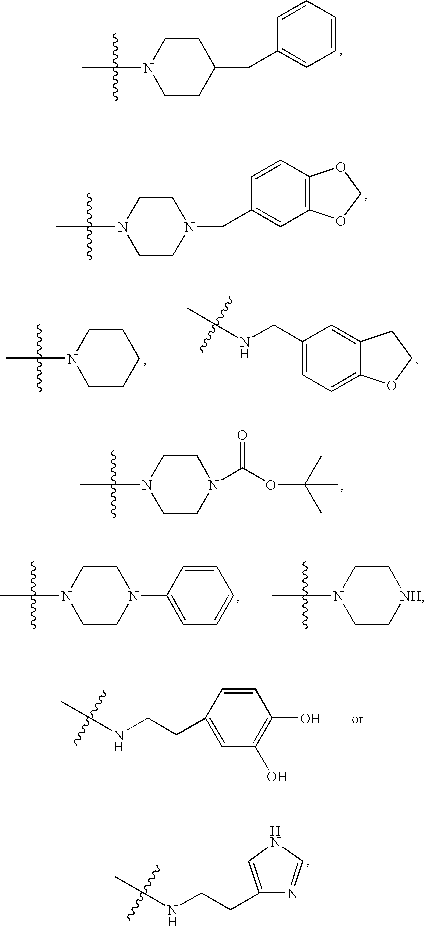
under conditions sufficient to form a compound of the formula: 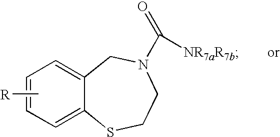
(F-2) reacting an amine of formula HNR₇ₐR_{7b} wherein NR₇ₐR_{7b} is as defined herein with triphosgene to form a compound of formula Cl₃CO(C═O)NR₇ₐR_{7b}, and reacting that compound with the starting compound 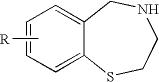
to form a compound of the formula: 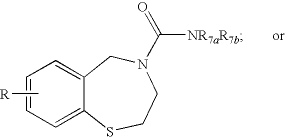
(G-2) forming the compound 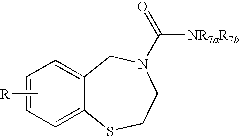
by the method of (E-2) or (F-2), wherein NR₇ₐR_{7b} is 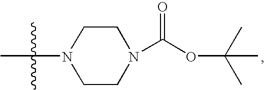
and reacting that compound with a Lawesson's Reagent under conditions sufficient to form a compound of the formula 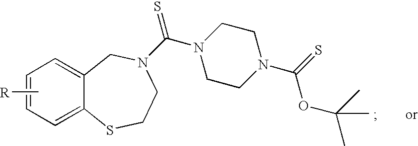
(H-2) forming the compound 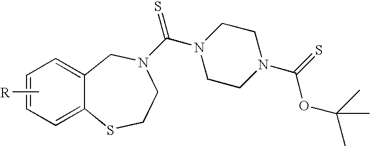
by the method of (G-2) and reacting that compound with an acid under conditions sufficient to form a compound of the formula: 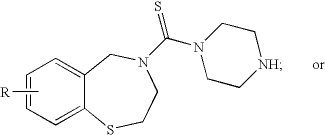
(I-2) reacting the starting compound 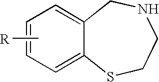
with Boc₂O or Boc-Cl under conditions sufficient to form a compound of the formula: 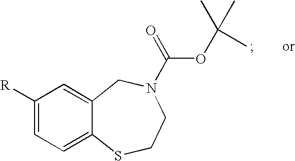
(J-2) forming the compound 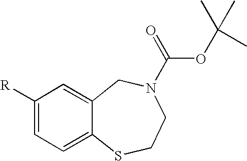
by the method of (I-2) and reacting that compound wherein R is methoxy, with BBr₃ under conditions sufficient to form a compound of the formula: 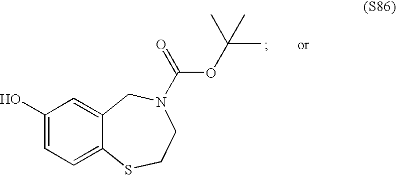
(K-2) forming the compound S86 by the method of (J-2) and reacting that compound with trifluoromethylsulfonyl anhydride, under conditions sufficient to form the compound S87 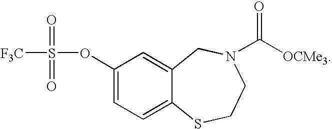
(L-2) forming the compound S87 by the method of (K-2) and reacting that compound with tris(dibenzylideneacetone)dipalladium(0), 2-(di-tert-butylphosphino)-biphenyl, and K₂PO₃, under conditions sufficient to form compound S88 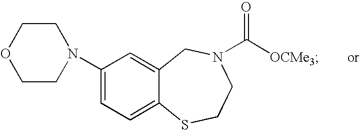
(M-2) forming the compound S87 by the method of (K-2) and reacting that compound with benzenethiol and a catalyst, under conditions sufficient to form compound S89 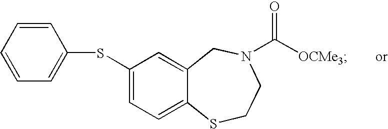
(N-2) forming the compound S87 by the method of (K-2) and reacting that compound with a base, phenylboronic acid, and a catalyst, under conditions sufficient to form compound S90 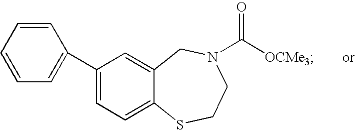
(O-2) forming the compound S87 by the method of (K-2) and reacting that compound with propene in the presence of a metal catalyst, a ligand and a base, under conditions suitable to form the compound S91 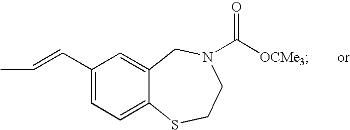
(P-2) forming the compound S87 by the method of (K-2) and reacting that compound with zinc cyanide and a catalyst, under conditions sufficient to form compound S92 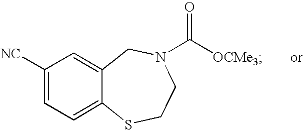
(Q-2) forming the compound S87 by the method of (K-2) and reacting that compound with benzylamine in the presence of a catalyst, a ligand and a base, under conditions suitable to form the compound S93 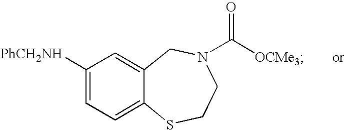
(R-2) forming the compound S86 by the method of (J-2) and reacting that compound with acetic anhydride, under conditions sufficient to form compound S94 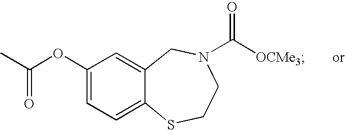
(S-2) forming the compound S94 by the method of (R-2) and reacting that compound with AlCl₃, under conditions sufficient to form compound S95 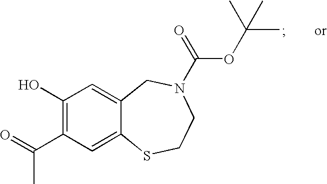
(T-2) forming the compound S86 by the method of (J-2) and reacting that compound with NaI and Chloramine-T, under conditions sufficient to form compound S96 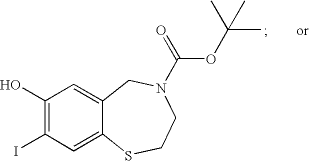
(U-2) forming the compound S86 by the method of (J-2) and reacting that compound with H₂SO₄ to form a sulfuric acid mixture: and then adding HNO₃ to the sulfuric acid mixture thus formed under conditions sufficient to form compound S97 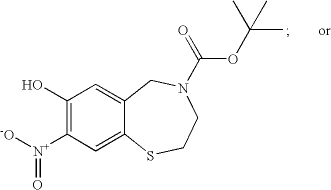
(V-2) forming the compound S97 by the method of (U-2) and reacting that compound and hydrogenating that compound under conditions sufficient to form compound S98 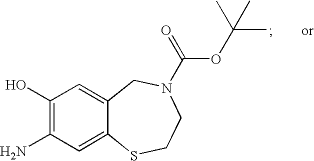
(W-2) forming the compound S98 by the method of (V-2) and reacting that compound with sodium nitrite and NaBaF₄ under conditions sufficient to form compound S99 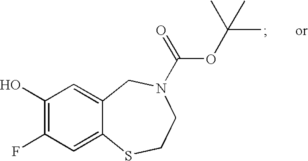
(X-2) forming the compound S98 by the method of (V-2), dissolving that compound in an acidic solution to form a mixture and then reacting the mixture with sodium nitrite and NaN₃ under conditions sufficient to form compound S100 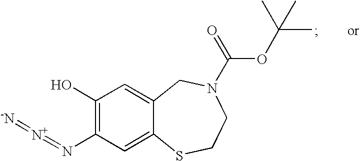
(Y-2) reacting the starting compound 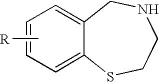
with a compound of formula 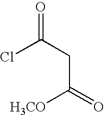
to produce a compound of formula: 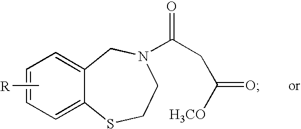
(Z-2) forming the compound 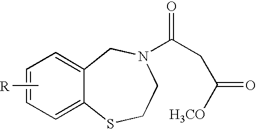
by the method of (Y-2) and reacting that compound with an acid or base under conditions sufficient to form a compound of the formula: 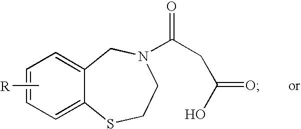
(AA) reacting the starting compound 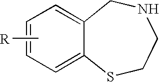
with formaldehyde (CH₂O) and a reducing agent under conditions sufficient to form a compound of the formula: 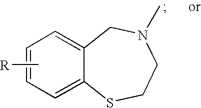
(BB) forming the compound 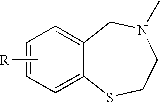
by the method of (AA) and reacting that compound with CH₃X₃, under conditions sufficient to form a compound of the formula: 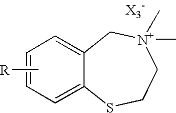
wherein X₃ is a halogen selected from F, Cl, Br and I; or
(CC) forming the compound 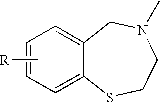
by the method of (AA) and reacting that compound with hydrogen peroxide, under conditions sufficient to form a compound of the formula:

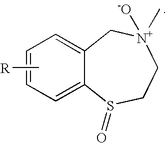

19. The method of claim 18, wherein R is methoxy and is located at position 7 of the benzothiazepine ring, or wherein the reducing agent in (A-2) is sodium cyanoborohydride (NaBCNH₃) or sodium triacetoxyborohydride.

20. A method of synthesis of organic compounds from the starting compound 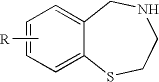
wherein R is H, OR₁, SR₁, N(R₁)₂ alkyl or halide; and R₁ is independently at each occurrence alkyl, aryl, or H, which comprises:
(A-3) reacting the starting compound 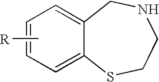
with N-benzyloxycarbonyl-glycine (Cbz-Gly), under conditions sufficient to form a compound of the formula: 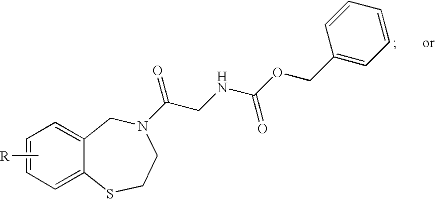
(B-3) forming the compound 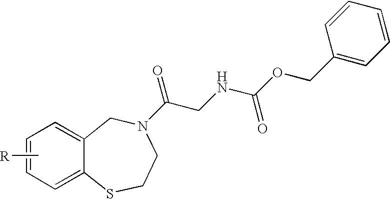
by the method of (A-3) and treating that compound with an acid under conditions sufficient to form a compound of the formula: 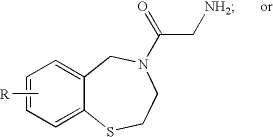
(C-3) reacting the starting compound 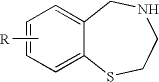
with a compound of formula: 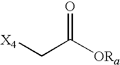
wherein X₄ is a halogen or a sulfonate, and Rₐ is a C₁-C₄ alkyl under conditions sufficient to form a compound of the formula: 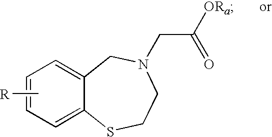
(D-3) forming the compound′ 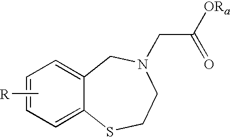
by the method of (C-3) and treating that compound with an acid or a base under conditions sufficient to form a compound of the formula: 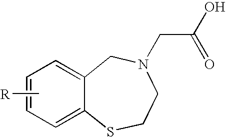
or a salt thereof, or
(E-3) reacting the starting compound 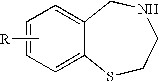
with BODIPY TMR-X under conditions sufficient to form a compound of the formula: 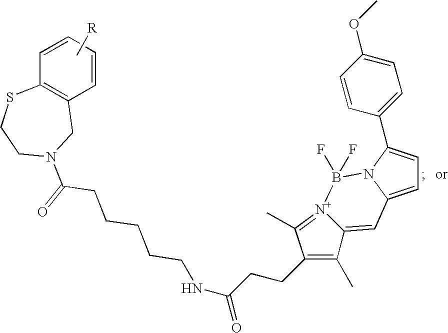
(F-3) reacting the starting compound 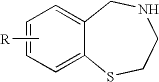
with 4-OH-benzyl bromide or benzyl bromide under conditions sufficient to form a compound of the formula:
wherein R₁₈ is OH or H; or 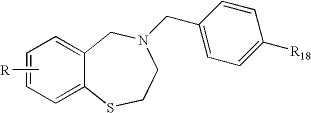
(G-3) reacting the starting compound 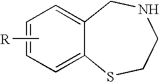
with DIEA and then with acetoxyacetyl chloride, under conditions sufficient to form a compound of the formula: 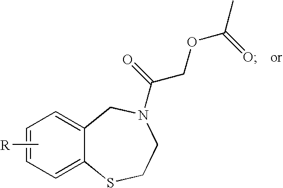
(H-3) forming the compound 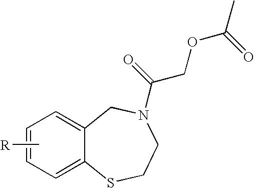
by the method of (G-3) and reacting that compound under conditions sufficient to form a compound of the formula: 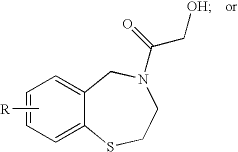
(I-3) reacting the starting compound 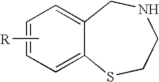
with a compound of formula C₆H₄—NCX₅, wherein X₅ is O or S, under conditions sufficient to form a compound of the formula: 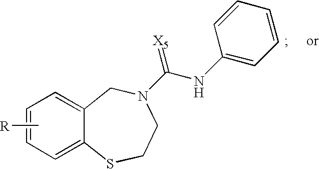
(J-3) reacting the starting compound 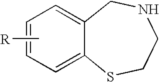
with phenyl methoxyphosphonyl chloride (Ph(MeO)P(O)Cl) in a solvent to form a reaction mixture; removing the solvent from the reaction mixture to form a residue; and purifying and separating the product into the isomers of compounds of the formulae: 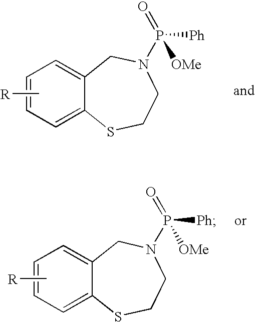
(K-3) reacting the starting compound 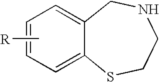
with a compound of formula ClOC—X₆—COCl, wherein X₆ is —CH₂—CH₂— or 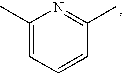
under conditions sufficient to form a compound of the formula: [chemical structure]
